(19) United States
(12) Patent Application Publication
(10) Pub. No.: US 2026/0315655 A1
(43) Pub. Date: Oct. 15, 2026

(54) COMPOSITIONS AND METHODS COMPRISING DECELLULARIZED EXTRACELLULAR MATRIX

(71) Applicant: YALE UNIVERSITY, New Haven, CT (US)

(72) Inventors: Themis Kyriakides, New Haven, CT (US); Zhuoyue Chen, New Haven, CT (US)

(21) Appl. No.: 19/474,017

(22) PCT Filed: Apr. 12, 2024
(86) PCT No.: PCT/US2024/024208

§ 371 Date: Oct. 9, 2025

Related U.S. Application Data
(60) Provisional application No. 63/495,745, filed on Apr. 12, 2023.

(51) Int. Cl. A61L 27/36; A61L 27/52

(52) U.S. Cl. CPC A61L 27/3633; A61L 27/3608; A61L 27/3683; A61L 27/3687; A61L 27/3691; A61L 27/52

(57) ABSTRACT

The present invention relates to a decellularized extracellular matrix (ECM), wherein the ECM originates from a tissue lacking thrombospondin-2 (TSP2). The invention further relates to compositions and methods for promoting tissue regeneration in a subject by administering a decellularized extracellular matrix (ECM), wherein the ECM originates from a tissue lacking thrombospondin-2 (TSP2). The invention further provides methods for preparing a decellularized extracellular matrix (ECM), wherein the ECM originates from a tissue lacking thrombospondin-2 (TSP2).

### CROSS REFERENCE TO RELATED APPLICATIONS

This application is entitled to priority under 35 U.S.C. § 119 (e) to U.S. Provisional Patent Application No. 63/495,745, filed Apr. 12, 2023, the contents of which are hereby incorporated by reference in its entirety herein.

### STATEMENT REGARDING FEDERALLY SPONSORED RESEARCH OR DEVELOPMENT

This invention was made with government support under DK115969 awarded by National Institutes of Health. The government has certain rights in the invention.

### SEQUENCE LISTING

The instant application contains a Sequence Listing which has been submitted in XML format via Patent Center and is hereby incorporated by reference in its entirety. Said XML copy, created on Mar. 4, 2024, is named 047162-7321WO1.xml and is 27,438 bytes in size.

### BACKGROUND OF THE INVENTION

Regenerative material requires use of biocompatible scaffolds, examples of which include synthetic polymers and decellularized extracellular matrix (ECM). ECM is a complex network of materials, such as proteins and polysaccharides, that are secreted locally by cells and remain closely associated with them. This non-cellular network of materials is present within all tissues and organs, and provides not only essential physical scaffolding for the cellular constituents, but also initiates crucial biochemical and biomechanical cues that are required for tissue morphogenesis, differentiation and homeostasis.

Advantages of decellularized ECM over synthetic materials include: existence of native ECM structure, retention of matrix-bound growth factors and other bioactive components, and a favorable host response. Nevertheless, decellularized materials are not without limitations. While synthetic materials can be engineered to fit almost any need, decellularized materials can be difficult to customize, because they rely on a natural source (either animal tissues or cells grown in vitro).

Thrombospondin-2 (TSP2) is an anti-angiogenic, matricellular protein that interacts with ECM proteins and with a variety of cell surface receptors including CD36, CD47, heparin sulfate proteoglycan, low-density lipoprotein receptor-related protein, and oBs. The phenotype of TSP2 knock-out (TSP2KO) mice is dominated by abnormalities in connective tissue and a platelet aggregation defect that manifests an abnormal bleeding tendency. ECM lacking TSP2 (“TSP2-null” or “TSP2KO” ECM) derived from cells or tissue has been used to generate tunable hydrogels (see, WO 2019/083842).

Hydrogels are materials composed of polymers swollen with water and can be fabricated with synthetic or natural starting materials. Hydrogels formulated from natural sources are attractive because they should maintain a level of biochemical complexity not achievable with purified polymers.

There remains a need in the art for decellularized ECM, methods of preparing the decellularized ECM, and its use for regenerative medicine. The present invention addresses this need.

### SUMMARY OF THE INVENTION

In one aspect provided herein is a method for promoting tissue regeneration in a subject in need thereof, the method comprising administering to the subject a decellularized extracellular matrix (ECM) of a tissue, wherein the tissue lacks functional thrombospondin-2 (TSP2). In another aspect provided herein is a decellularized extracellular matrix (ECM) of a tissue, wherein the tissue lacks functional thrombospondin-2 (TSP2). In another aspect provided herein is a method for preparing a decellularized extracellular matrix (ECM), the method comprising: (i) obtaining or having obtained a tissue lacking functional thrombospondin-2 (TSP2) from a mammal; and (ii) decellularizing the tissue to generate the decellularized ECM.

### BRIEF DESCRIPTION OF THE DRAWINGS

The following detailed description of specific embodiments of the invention will be better understood when read in conjunction with the appended drawings. For the purpose of illustrating the invention, specific embodiments are shown in the drawings. It should be understood, however, that the invention is not limited to the precise arrangements and instrumentalities of the embodiments shown in the drawings.

FIGS. 1A-1M show preparation and characterization of muscle-derived ECM hydrogels. FIG. 1A: a series of photographs of various processing stages from freshly isolated muscle tissue to final ECM hydrogel product for muscle tissue from WT and TSP2KO mice. The muscle ECM was lyophilized, and the resulting hydrogel concentration was 10 mg ECM/mL. FIG. 1B: histologically stained sections from various processing stages of freshly isolated muscle tissue to the ECM after Triton/SDC treatment. H&B and Trichrome staining each shows cumulative decellularization of the muscle tissue with each processing step and the subsequent retention of collagen proteins. FIG. 1C: Quantification of DNA content for fresh muscle, ECM (“decell”), and final ECM hydrogel product for muscle tissue from WT and TSP2KO mice. The cell's DNA content was tested by the DNeasy Kit and NanoDrop and a significant difference was detected after decellularization for both WT and TSP2KO samples (***p<0.001, n=5). FIG. 1D: The proteins in each step of decellularizing mouse muscle were tested by SDS-PAGE. FIG. 1E: Ponceau (Left) and Western Blot (Right) proved TSP2 was present in the WT ECM, however, it was absent within the TSP2KO ECM. FIG. 1F: Western Blot indicated the presence of Col I for both WT and TSP2KO derived ECM hydrogels. FIG. 1G: SEM images for muscle-derived ECM hydrogel microstructure. FIG. 1H: The fibril diameter in the hydrogels was determined by SEM. Quantification of the diameter of the WT and TSP2KO hydrogels indicated that their diameter had no significant difference. FIG. 1I: The frequency distribution of the fibril curvature in the hydrogels was determined by SEM. Quantification of the curvature of the WT and TSP2KO hydrogels indicated the average curvature of TSP2KO fibrils is higher than that of WT fibrils (*p<0.05, n=6). FIG. 1J: Rheometer testing of the storage modulus and loss modulus for both hydrogels at 10 mg/mL. Analysis of storage modulus data for muscle-derived ECM hydrogels illustrating that TSP2KO ECM hydrogels display reduced biomechanical properties compared to WT ECM hydrogels. Rheometer testing of storage modulus and loss modulus for 10 mg/ml muscle-derived ECM hydrogel. The storage modulus of WT group indicated that the WT hydrogel was significantly more elastic than the TSP2KO hydrogel. *p<0.05 (n=6). FIG. 1K: Collagen abundance from quantitative proteomics was found to differ between WT and TSP2KO mouse muscle ECM. FIG. 1L: A volcano plot demonstrated differences between genotypes. FIG. 1M: The top 26 most abundant ECM proteins showed differences between WT and TSP2 KO muscle hydrogels. Results are given as mean±SEM, n=3, *p<0.05.

FIGS. 2A-2M show data characterizing cell-hydrogel interactions. FIG. 2A: Representative SEM images of C2C12 cells seeded on WT or TSP2KO hydrogels for 2 hrs and 4 hrs. FIG. 2B: Cells on TSP2KO hydrogels for 4 hrs exhibited larger area and perimeter and were more elongated. One-way ANOVA with post hoc Turkey HSD test, n=50 cells, ***p<0.001. FIG. 2C: Representative images of H&E-stained sections following seeding of cells on hydrogels in a transwell system for 72 hr. FIG. 2D: Cell invasion was measured at the indicated time points and found to be greater in TSP2KO hydrogels at 72 hr (*p<0.05, n=3). FIG. 2E: C2C12 muscle cells were cultured for 5 days on either WT or TSP2KO derived hydrogels. Nuclei were stained with DAPI, and the cytoskeleton was stained with rhodamine-phalloidin. FIG. 2F: C2C12 muscle cell proliferation was analyzed every day for 5 days using CCK-8. Analysis of the proliferation assay indicated that the WT and TSP2KO mice muscle hydrogel did not affect cell proliferation, indicating that both muscle hydrogels are not toxic to the cells. The number of cells on the hydrogels has no significant difference between WT and TSP2KO group at each time point (p>0.05, n=12). FIG. 2G: NIH3T3 fibroblasts were cultured for 5 days on either WT or TSP2KO derived hydrogels. Nuclei were stained with DAPI, and the cytoskeleton was stained with rhodamine-phalloidin. FIG. 2H: NIH3T3 fibroblasts proliferation was analyzed every day for 5 days using CCK-8. Analysis of the proliferation assay indicated that the WT and TSP2KO mice muscle hydrogel did not affect cell proliferation, indicating that both muscle hydrogels are not toxic to the cells. The number of cells on the hydrogels has no significant difference between WT and TSP2KO group at each time point (p>0.05, n=12). FIG. 2I: C2C12 cells invasion in the hydrogels. The frequency distribution of C2C12 cells invasion distance in the hydrogels is indicated by H&E staining. FIG. 2J: NIH3T3 cells invasion in the hydrogels. The frequency distribution of NIH3T3 cells invasion distance in the hydrogels is indicated by H&E staining. FIG. 2K: NIH3T3 cells invasion in 2 mg/mL hydrogels was significantly greater for TSP2KO hydrogels than for WT hydrogels at 12 hours (*p<0.05, n=3). FIG. 2L: Representative images of 2 mg/ml WT and TSP2KO hydrogels formed on 6-well plates. FIG. 2M: NIH3T3 cells were seeded and cultured on a 2 mg/ml WT or TSP2KO derived hydrogel for 2 hours. SEM analysis of the 2 mg/ml hydrogels indicated that the ECM has a bigger pore size compared to the 10 mg/mL hydrogels. Pseudocolor was employed to visualize the interaction of the ECM matrix and the NIH3T3 fibroblasts. The results indicate that the fibroblasts successfully migrated into the gel within 2 hours. Moreover, the TSP2KO derived gel provided a structure that allowed for a more thorough fibroblast integration into the gel as opposed to the WT derived gel.

FIGS. 3A-3G show data characterizing cell-hydrogel interactions. FIG. 3A: Fluorescence microscopy images of grouped proliferation assay of HUVEC on muscle hydrogels. FIG. 3B: Quantification of grouped proliferation assay of HUVEC on muscle hydrogels indicated that both WT and TSP2KO mice muscle hydrogel did not affect cell proliferation (p>0.05, n=12). FIG. 3C: Representative merged fluorescent images of HUVECs cultured on hydrogels for 24 hours and stained with DAPI (nuclei), and rhodamine-phalloidin (actin cytoskeleton). FIG. 3D: Analysis of tube diameter for TSP2KO and WT hydrogels. FIG. 3E: Image analysis indicated that tube formation was greater on TSP2KO hydrogels than WT hydrogels (*p<0.05, n=4). FIG. 3F: Fluorescence microscopy was employed to visualize the morphology of the macrophages cultured on the muscle hydrogels for 3, 7 and 14 days. FIG. 3G: qRT-PCR indicated that the gene expression between WT gel cultured macrophages and TSP2KO gel cultured macrophages had no significant difference (p>0.05, n=3).

FIGS. 4A-4J show data for the evaluation of WT and TSP2KO muscle-derived ECM hydrogels biocompatibility in subcutaneous injection model. FIG. 4A: Representative images of H&E-stained sections of WT and TSP2KO hydrogels injected SC in WT mice for 5 or 10 days. FIG. 4B: Image analysis of hydrogel area revealed that the TSP2KO hydrogels formed more elongated shape compared to WT (**p<0.001, n=5). FIG. 4C: Image analysis revealed that TSP2KO gel contained more cells at day 5 in comparison to WT (*p<0.05, n=5). FIG. 4D: H&E staining of the samples implanted into mice subcutaneously after 10 days. FIG. 4E: Immunochemical staining (Neurofilament H) indicated that the TSP2KO hydrogel had nerves present 10 days after subcutaneous injection in mice but no nerve in the WT hydrogel. FIG. 4F: Immunochemical staining (α-SMA and CD31) indicated that the TSP2KO hydrogel had blood vessels present 10 days after subcutaneous injection in mice. Asterisks (☆) represent the hydrogel. FIG. 4G: Representative images of day 5 and day 10 hydrogel sections stained with Vimentin (Fibroblast/mesenchymal cells), F4/80 (macrophages), or CD3e (T cells) and visualized with the peroxidase reaction. FIG. 4H: Image analysis revealed increased Vimentin-positive cells on day 5 and 10, no differences in F4/80-positive cells, and increased CD3e+ cells on day 5 in TSP2KO hydrogels. FIG. 4I: Representative images of WT and TSP2KO hydrogels injected SC in WT mice for 10 days and stained with antibodies to detect neurofilament H as a marker for innervation. In contrast to TSP2KO, no innervation was detected in WT hydrogels. FIG. 4J: Representative single and merged immunofluorescence images show the presence of newly formed vessels in day 10 TSP2KO hydrogels and absence of newly formed vessels in day 10 WT hydrogels (**p<0.01 at day 5, *p<0.05 at day 10, n=5).

FIGS. 5A-5G show data for VML model and treatment with muscle-derived ECM hydrogel. Where present, stars (*) represent the hydrogel. FIG. 5A: VML surgery procedure. A scalpel was used to make a 2 mm deep cut around the stencil removing a 2×7×2 mm piece of the muscle. After creating the VML, the hydrogel was implanted directly into the injury. FIG. 5B: Representative images of WT and TSP2KO hydrogels 3 days and 14 days after application to injured muscles and stained with Masson's trichrome, H&E, antibodies to detect Laminin, Vimentin, F4/80, or CD3e. FIG. 5C: Tissue samples from the VML model mice were obtained on day 3, day 14 or day 28 and imaged via Trichrome staining. Most of the blue areas represent the hydrogel proteins. The hydrogel almost disappeared 28 days after injection. Newly formed muscles were observed in the TSP2KO hydrogel on day 14. FIG. 5D: Quantification of the area of the hydrogel calculated using ImageJ (*p<0.05, n=3). FIG. 5E: Quantification of the number of cells analyzed using ImageJ. There was no significant difference in the presence of fibroblast between the TSP2KO and WT gels for each time point (p>0.05, n=3). FIG. 5F: Quantification of the number of cells analyzed using ImageJ. There were fewer macrophages present in the TSP2KO gel as compared to the WT gel at day 14 (*p<0.05, n=3), FIG. 5G: Quantification of the number of cells analyzed using ImageJ. No T cells were present in either of the hydrogels 3 days after transplantation. At day 14, the total presence of T cells did not differ significantly for either hydrogel (p>0.05, n=3).

FIGS. 6A-6J show data related to detection of innervation and angiogenesis in the VML model. FIG. 6A: Representative images of injured muscles treated with WT or TSP2KO hydrogels for 14 days and stained with antibodies to detect α-SMA, or CD31 as markers of neovessel formation. FIG. 6B: Quantification of neovessel formation by image analysis revealed an increase in muscles treated with TSP2KO hydrogel at day 14 (**p<0.01, n=5). FIG. 6C: Representative images of muscles treated with WT or TSP2KO hydrogels for 14 days and stained with antibodies to detect neurofilament H as a marker for innervation. In contrast to TSP2KO hydrogels, no innervation was detected in WT hydrogels.

FIG. 6D: Representative images of muscle treated with WT or TSP2KO hydrogels for 14 days and stained with antibodies of Laminin and PAX7 to detect remodeling and skeletal muscle satellite cells, respectively. FIG. 6E: A western blot analysis of the newly formed tissue in VML model indicated the presence of neurofilament H in each specimen. The integrated density of the protein band was analyzed by Image J. At day 7, there was more neurofilament H expressed in the newly formed tissue from TSP2KO hydrogels than that of the WT hydrogel (*p<0.05, n=3). FIG. 6G: Quantification of the ambulation distance of the mice before and after the VML surgery. Nine mice were traced. FIG. 6H: Quantification of the max running speed of the mice before and after the VML surgery. Nine mice were traced. FIG. 6I: Mice were monitored by video for voluntary ambulation during a 6 min period. Immediately post-op, TSP2KO mice exhibited increased voluntary ambulation (*p<0.05, n=9). FIG. 6J: Max running speed of mice before and after injury and treatment with WT or TSP2KO hydrogels. Mice treated with TSP2KO hydrogels were protected from injury and achieved higher running speed by day 14 (*p<0.05, n=9).

FIG. 7 is a schematic diagram of the preparation process for muscle-derived ECM hydrogel and in vivo and in vitro applications thereof.

FIGS. 8A-8H show data for the preparation and characterization of bone matrix hydrogel. FIG. 8A: A series of photographs illustrating the process of preparing bone matrix hydrogel. FIG. 8B: From the H&E observation, the cells are removed from bone step by step. At final, the collagen is left in the bone ECM which can be observed in the Trichrome staining. FIG. 8C: The cell's DNA content was tested by the DNeasy Kit and NanoDrop and a significant difference was detected after decellularization for both WT and TSP2KO samples (***p<0.001, n=6). FIG. 8D: Western Blot indicated that TSP2 was present in the WT mice bone but absent within the TSP2KO mice bone. FIG. 8E: The proteins in each step of decellularizing mouse bone were analyzed by SDS-PAGE. FIG. 8F: SEM observation of the bone matrix hydrogel (6 mg/ml). In both WT and TSP2KO hydrogels, collagen self-assembles into nanofibers. The average curvature of TSP2KO fibrils is higher than that of WT fibrils. N=180 fibrils in each group, 60 fibrils per sample, n=3 samples in each group, *p<0.05. FIG. 8G: Analysis of the diameter of fibrils of the WT and TSP2KO hydrogels indicated that their diameter had no significant difference. N=300 fibrils in each group, 100 fibrils per sample, n=3 samples in each group, p>0.05. FIG. 8H: Modulus analysis of the bone hydrogel using Rheometer. The storage modulus of WT hydrogel is significantly higher than that of TSP2KO hydrogel (*p<0.05, n=4).

FIGS. 9A-9F show data from grouped attachment assay of mouse and human MSCs on bone hydrogels. FIG. 9A: SEM images of mouse MSCs on WT or TSP2KO hydrogels. FIG. 9B: SEM images of human MSCs on WT or TSP2KO hydrogels. FIG. 9C: Representative images of mouse MSCs cultured for 2 hours and stained with phalloidin (cytoskeleton) and DAPI (nucleus), FIG. 9D: Quantification of mouse MSCs area, perimeter, circularity, and elongation index (one-way ANOVA with post hoc Tukey HSD test, n=50 cells, *p<0.05, **p<0.01, ***p<0.001). Error bars represent standard error of the mean. FIG. 9E: Representative images of human MSCs cultured for 2 hours and stained with phalloidin (cytoskeleton) and DAPI (nucleus). FIG. 9F: Quantification of human MSCs area, perimeter, circularity, and elongation index (one-way ANOVA with post hoc Tukey HSD test, n=50 cells, *p<0.05, **p<0.01, ***p<0.001). Error bars represent standard error of the mean.

FIGS. 10A-10E show data for MSC attachment and proliferation on the WT and TSP2KO bone ECM hydrogels. FIG. 10A: SEM images were employed to assess the human MSCs attachment for the WT and TSP2KO hydrogels. FIG. 10B: Mouse MSCs cultured for 5 days on either WT or TSP2KO derived hydrogels. The nuclei are stained with DAPI, and the cytoskeleton stained with rhodamine-phalloidin. FIG. 10C: Human MSCs cultured for 5 days on either WT or TSP2KO derived hydrogels. The nuclei are stained with DAPI, and the cytoskeleton stained with rhodamine-phalloidin. FIG. 10D: Quantification of the proliferation assay results for mouse MSCs indicated that TSP2KO mice hydrogel could promote mouse MSCs proliferation (*p<0.05, n=3). FIG. 10E: Quantification of the proliferation assay results for human MSCs indicated that TSP2KO mice hydrogel could promote human MSCs proliferation (*p<0.05, n=3).

FIGS. 11A-11G show data for mouse and human MSC invasion and HUVEC tube formation on the WT and TSP2KO hydrogels. FIG. 11A: H&E stained sections of mouse MSC invasion in WT and TSP2KO hydrogels. FIG. 11B: H&E stained sections of human MSC invasion in WT and TSP2KO hydrogels. FIG. 11C: Quantification of mouse MSC invasion distance shows a significant difference in MSCs invasion on day 3 (*p<0.05, n=3). FIG. 11D: Quantification of human MSC invasion distance shows a significant difference in MSCs invasion on day 3 (*p<0.05, n=3). FIG. 11E: Fluorescence imaging of HUVECs seeded on WT or TSP2KO hydrogels. Nuclei are stained with DAPI and the cytoskeleton stained with rhodamine-phalloidin. FIG. 11F: Quantification of tube diameter of the formed tubes showed no significant difference between WT and TSP2KO groups (p>0.05, n=4). FIG. 11G: Quantification of the formed tubes showed that the TSP2KO hydrogel produced more tubes than the WT hydrogel (*p<0.05, n=4).

FIGS. 12A-12C show data for MSC invasion in the WT and TSP2KO hydrogels. FIG. 12A: Quantification of the frequency distribution of mouse and human MSC invasion distance in WT and TSP2KO hydrogels as indicated by H&E staining. FIG. 12B: H&E stained sections of human MSCs seeded on WT or TSP2KO hydrogel. FIG. 12C: Quantification of the human MSC invasion showed a significant difference between WT and TSP2KO at 12 hours (*p<0.05, n=3).

FIGS. 13A-13F show data for osteogenic differentiation of MSCs on WT and TSP2KO hydrogels. FIG. 13A: qRT-PCR indicated that the osteogenic-gene expression between WT gel cultured mouse MSCs and TSP2KO gel cultured mouse MSCs had no significant difference (p>0.05, n=4). FIG. 13B: Representative Western blots of Osteopontin (OPN) and Osteocalcin (OCN) expression in lysates collected 3-, 7- and 14-day post-culturing of mouse MSCs on WT and TSP2KO hydrogels and quantification thereof.

FIG. 13C: qRT-PCR indicated that the osteogenic-gene expression between WT gel cultured human MSCs and TSP2KO gel cultured human MSCs had no significant difference (p>0.05, n=4). FIG. 13D: Representative Western blots of Osteopontin (OPN) and Osteocalcin (OCN) expression in lysates collected 3-, 7- and 14-day post-culturing of human MSCs on WT and TSP2KO hydrogels and quantification thereof. FIG. 13E: Representative fluorescence images of OPN and OCN staining for mouse MSCs on WT and TSP2KO hydrogels at 3, 7 and 14 days. FIG. 13F: Representative fluorescence images of OPN and OCN staining for human MSCs on WT and TSP2KO hydrogels at 3, 7 and 14 days.

FIGS. 14A-14C show data related to analysis of the newly formed tissues in the calvarial defect model. FIG. 14A: Micro-CT scan of implanted WT hydrogels, TSP2KO hydrogels and Control (without implant) skulls. FIG. 14B: After implantation of the hydrogels, the percentage of the new bone formation for TSP2KO hydrogel was significantly improved compared to that in the non-implanted control group at weeks 4, 8 and 12 (*p<0.05, n=5), and compared to the WT hydrogel group at week 12 (*p<0.05, n=5). FIG. 14C: H&E stained sections of the newly formed tissues.

FIG. 15 illustrates IHC analysis of the skulls in the critical-size calvarial defect mouse model. Immunochemistry staining for Col I and OCN evaluated the ECM deposition and the regenerated bone tissue. The shading represents Col I or OCN in the immunostained images.

FIGS. 16A-16B show data for histological analysis of the newly formed bone and cartilage. FIG. 16A: Trichrome staining analysis of the bone healing with WT hydrogel in comparison to TSP2KO hydrogel group and control group. FIG. 16B: Saffranin O/Fast Staining analysis of the chondrocytes in the newly formed tissues. Arrows indicate chondrocyte and arrows indicate newly formed bone.

FIGS. 17A-17C show data for immunofluorescence analysis of the blood vessels in the newly formed tissues. FIG. 17A: Immunofluorescence staining for PECAM-1 (CD31) and alpha smooth muscle actin (α-SMA) shows microvascular endothelium in adventitia and outer vessel wall. FIG. 17B: Quantification of blood vessels indicated that there were more blood vessels present in the newly formed tissue in the TSP2KO hydrogel as compared to the WT hydrogel post-surgery 4 weeks (*p<0.05, n=3). FIG. 17C: Quantification of the diameter of blood vessels indicated that there was no significant difference between WT and TSPKO group (p>0.05, n=3).

FIG. 18 is a schematic diagram of the preparation process for bone-derived ECM hydrogel and in vivo and in vitro testing thereof.

FIG. 19 shows the localization and representative histology of the three types of cartilages. Hyaline cartilage is located on the articular surface of major bones, as well as larynx, trachea and rib cage sternum. Fibrocartilage comprises intervertebral disk and knee meniscus. Elastic cartilage is present in the ear.

FIG. 20 is a proposed workflow for pig cartilage decellularization.

FIG. 21 is a proposed workflow for generating cartilage dECM derived hydrogels.

FIG. 22 shows demonstration of successful pig ear cartilage decellularization and hydrogel fabrication. FIG. 22A-FIG. 22B examine H&E staining images before and after decellularization show a significantly decreased nuclear content, identified by the dimmer purple color and decreased nuclei count in the cartilage. FIG. 22C shows quantification of ear cartilage layer thickness shows a higher thickness for WT, despite sex difference. FIG. 22D shows DNA extraction results before and after decellularization shows a reduction in DNA content to below 50 ng/mg tissue for both genotypes, which is an indicator for successful decellularization. FIG. 22E examines representative pictures of neutralized hydrogels from both genotypes inside the 37° C. incubator post neutralization. The color (given by phenol red mixed with 10×PBS) shows that gel pH is close to the titration endpoint. FIG. 22F shows representative pictures of crosslinked and fixed hydrogel pieces.

FIG. 22G analyzes BCA assay result of pregels for total protein content measurement. Pregels are prepared from 10 mg dECM/ml digestion solution. TKO pregel has a slightly higher overall protein content than WT.

FIGS. 23A-23D examines representative SEM images of hydrogels derived from FIG. 23A WT and FIG. 23B TKO pig ears at different magnifications. FIG. 23C-FIG. 23D analyze quantification on SEM images shows WT dECM fibers have higher diameters but similar curvature compared to TKO.

FIGS. 24A-24E show preliminary rheometry results on WT and TKO pig ear hydrogels, FIG. 24A shows strain sweep on gel shows a defined region where WT complex viscosity is larger than TKO. FIGS. 24B-24C shows temperature sweep on both genotypes shows gelation behavior once temperature reaches 37° C. WT hydrogel has slightly slower gelation and higher maximum storage modulus. FIGS. 24D-24E show frequency sweep on both genotypes shows a storage modulus greater than the loss modulus, indicating gel-like behavior. The two hydrogels have different biomechanical properties.

FIG. 25 shows the workflow of experimental setups for cell-hydrogel interaction.

FIG. 26 shows the experimental approaches for evaluating cell-hydrogel interactions.

FIG. 27 shows the in vivo workflow. Briefly, under general anesthesia, hind limb skin surrounding the patella is cut open. Lateral articular surface of the trochlear groove is exposed by opening the joint capsule, and a full thickness injury is created with a 21G needle on the patellar groove of femur. The injury must penetrate to the subchondral bone to be considered ‘full thickness’. Following injury, the parapatellar skin is sutured to close the wound with or without treatment. Mice are treated with antibiotics and analgesics and monitored for dehydration post surgery.

FIG. 28 shows the workflow with WT mice only. There are negative control groups and defect groups. Within the defect group, there are 6 subgroups in total: defect with no treatment, neutralized WT and TKO pregel injection, pregel with PLGA+growth factor injection, and hyaluronic acid injection. The evaluation process would be similar to what is discussed previously.

### DETAILED DESCRIPTION OF THE INVENTION

In one aspect, the present invention provides a method of promoting tissue regeneration in a subject in need thereof by administering a decellularized ECM of a tissue, wherein the tissue lacks TSP2. In some embodiments, the tissue is from a mammal.

In another aspect, the present invention provides a decellularized ECM of a tissue, wherein the tissue lacks functional thrombospondin-2 (TSP2). Throughout the application, thrombospondin-2 can be represented with TSP2, THBS2, or Thbs2 and can be used interchangeably. In some embodiments, the tissue is from a mammal.

In another aspect, the present invention provides a composition comprising: (i) a hydrogel comprising a decellularized extracellular matrix (ECM) of a tissue, wherein the tissue lacks functional thrombospondin-2 (TSP2). In some embodiments, the tissue is from a mammal; and (ii) a pharmaceutically acceptable carrier.

In another aspect, the present invention provides a method of preparing a decellularized extracellular matrix (ECM), the method comprising decellularizing a tissue to generate the decellularized ECM, wherein the tissue lacks functional TSP2. In some embodiments, the tissue is from a mammal.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “allogeneic” refers to organs, tissues, or cells originating from different individuals of the same species.

By “alteration” is meant a change (increase or decrease) in the expression levels or activity of a marker or clinical indicator as detected by standard art known methods such as those described herein. As used herein, an alteration includes a 10%-100% change in measured levels (e.g., 10, 20, 30, 40, 50, 60, 75, 80, 85, 90, 95, 100%).

The term “autologous” with reference to an organ, tissue, or cell denotes that the organ, tissue, or cell originates from the same individual.

The term “biocompatibility” refers to the properties of materials, such as a medical device or an implant, device being biologically compatible by not eliciting unwanted local or systemic responses from a living system or tissue. In other embodiments, the device does not elicit any significantly and/or measurably deleterious responses from the living system or tissue. A biocompatible device is substantially non-toxic, non-injurious or non-inhibiting or non-inhibitory to cells, tissues, organs, and/or organ systems that would come into contact with the device, scaffold, composition, etc.

The term “coating” refers to a covering, layer or film, of a substance applied to the surface of a substrate. The coating may be an all-over coating, completely covering the substrate, or it may only cover parts of the substrate.

As used herein, the term “comminute” and any other word forms or cognates thereof, such as, without limitation, “comminuting”, refers to the process of reducing larger particles into smaller particles, including, without limitation, by grinding, blending, shredding, slicing, milling, or cutting. ECM can be comminuted while in any form, including, but not limited to, hydrated forms, frozen, air-dried, lyophilized, powdered, sheet-form.

The expression “difference in the level of” or “differentially present” refers to differences in the quantity and/or the frequency of a marker present in a sample taken from subjects having a disease as compared to a control subject. A marker can be differentially present in terms of quantity, frequency or both. A difference in the level of a polypeptide is present between two samples if the amount of the polypeptide in one sample is statistically significantly different from the amount of the polypeptide in the other sample. Alternatively or additionally, a polypeptide is differentially present between two sets of samples if the frequency of detecting the polypeptide in a diseased subjects' samples is statistically significantly higher or lower than in the control samples. A marker that is present in one sample, but undetectable in another sample is differentially present.

A “disease” is a state of health of an animal wherein the animal cannot maintain homeostasis, and wherein if the disease is not ameliorated then the animal's health continues to deteriorate. “Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, IRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” with reference to a material denotes that the material is from or produced inside an organism, cell, tissue or system.

As used herein, the term “exogenous” with reference to a material denotes that the material is introduced from or produced outside an organism, cell, tissue or system.

The term “expression” as used herein is defined as the transcription and/or translation of a particular nucleotide sequence driven by a promoter.

The terms “extracellular matrix” or “ECM” refer to proteins that are secreted by cells and assembled in a three dimensional manner to provide structural support for cells. Generally, extracellular matrix comprises proteins such as collagens (e.g. type I, III, IV, and V collagens), vitronectin, fibronectin, laminin, thrombospondin, entactin, and nidogen; and glycosaminogly cans and proteoglycans. However, it is noted that the extracellular matrix can vary in composition, and structural assembly, depending on its anatomic origin. In some instances, ECMs include an isolated basement membrane produced by vascular endothelial cells and a membrane on which the cells rest in vivo. Non limiting examples of ECMs are those originating from a tissue of a mammal, such as muscle (e.g., skeletal muscle, smooth muscle, or cardiac muscle), cartilage, a connective tissue, a tendon, a ligament, or bone. While matrices may differ somewhat in their composition, they are primarily composed of collagens (e.g. type I, III, IV, VI collagens), fibronectin, laminins, and other matricellular proteins. Despite the variation due to anatomic origin, extracellular matrix from any anatomie site could be useful in the present invention. Of particular interest in the present invention, are ECMs that comprise extracellular molecules that form a three-dimensional structure supporting cell and tissue growth. The ECMs of the present invention originate from a tissue lacking functional TSP2.

The term “hydrogel” as used herein refers to a gel in which the liquid component comprises water.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The term “immune response” as used herein is defined as a host response to an antigen that occurs when lymphocytes identify antigenic molecules as foreign and induce the formation of antibodies and/or activate lymphocytes to remove the antigen.

As used herein, the terms “immunosuppression” or “immunosuppressive therapy (IST)” involve an act that reduces the activation or efficacy of the immune system. Deliberately induced immunosuppression is performed to prevent the body from rejecting an organ transplant, treating graft-versus-host disease after a bone marrow transplant, or for the treatment of auto-immune diseases such as rheumatoid arthritis or Crohn's disease.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

As used herein, the phrases “lacks functional TSP2” and “lacking functional TSP2” with reference to a tissue denote that the expression of wild-type TSP2 or any functional variant thereof in the cells of the tissue is eliminated or significantly reduced compared to a wild-type tissue that is otherwise the same type of tissue. In some embodiments, a tissue lacking or that lacks functional TSP2 comprises cells which comprise one or both of (a) a TSP2-null knockout (KO) allele; and (b) suppressed TSP2 gene expression. TSP2 nucleic acid and/or amino acid sequence are disclosed by NCBI ascension numbers (NCBI Gene IDs: 7058 and 21826): NG_022911.2, NM_001381939.1, NM_001381940.1, NM_001381941.1, NM_001381942.1, NM_003247.5, NP_001368868.1, NP_001368869.1, NP_001368870.1, NP_001368871.1, NP_003238.2, NM_011581.3, and NP_035711.2, each of which is herein incorporated by reference in its entirety. In some embodiments, the TSP2 nucleic acid and/or amino acid sequence comprises a sequence as set forth in NG_022911.2, NM_001381939.1, NM_001381940.1, NM_001381941.1. NM_001381942.1, NM_003247.5, NP_001368868.1, NP_001368869.1, NP_001368870.1, NP_001368871.1, NP_003238.2, NM_011581.3, or NP_035711.2.

A TSP2 knock out allele or suppressed TSP2 gene expression can be obtained via a genetic engineering technique comprising a nuclease. Exemplary nucleases include, but are not limited to, a clustered regularly interspaced short palindromic repeats (CRISPR) associated nuclease, a transcription activator-like effector nuclease (TALEN), and a zinc-finger nuclease. A TSP2KO allele may comprise a non-functional TSP2 variant and/or may comprise a mutation or variant which eliminates expression of functional TSP2.

Alternatively, or in addition, cells of the tissue lacking functional TSP2 may comprise an inhibitory RNA molecule which suppresses TSP2 gene expression. Examples of such inhibitory RNA molecules include, but are not limited to, an RNA interference (RNAi) RNA, a short hairpin RNA (shRNA), a small interfering RNA (siRNA), a trans-acting siRNA (tasiRNA), a micro RNA (miRNA), an antisense RNA (asRNA), a long noncoding RNA (lncRNA), a CRISPR RNA (crRNA), a trans-activating crRNA (tracrRNA), a guide RNA (gRNA), a single guide RNA (sgRNA), a double-stranded RNA (dsRNA), a ribozyme, and any combination thereof.

By “marker” is meant any protein or polynucleotide having an alteration in level or activity that is associated with a disease or disorder.

By the term “modified” as used herein, is meant a changed state or structure of a molecule or cell of the invention. Molecules may be modified in many ways, including chemically, structurally, and functionally. Cells may be modified through the introduction of nucleic acids therein.

The term “model organism” refers to a non-human species that is easy to maintain and breed in a laboratory setting and has particular experimental advantages. Model organisms as used herein provide an in vivo model to research the effects of a human disease or condition and/or biological activities associated with a disease or condition, such as thrombosis.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

“Monitoring” refers to recording changes in a continuously varying parameter (e.g. monitoring progression of a disease).

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Parenteral” administration of an immunogenic composition includes, e.g., subcutaneous (s.c.), intravenous (i.v.), intramuscular (i.m.), or intrasternal injection, or infusion techniques.

The language “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent, lubricant; binder; disintegrating agent; wetting agent; emulsifier; coloring agent; release agent; coating agent; sweetening agent; flavoring agent; perfuming agent, preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

The terms “purified”, “biologically pure” or “isolated” as used herein mean having been increased in purity, wherein “purity” is a relative term, and not to be necessarily construed as absolute purity. For example, the purity of a substance, for example, but not limited to a nucleic acid, can be at least about 50%, can be greater than 60%, 70%, 80%, 90%, 95%, or can be 100%. The terms “purified”, “biologically pure” or “isolated” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

As used herein, “sample” or “biological sample” refers to anything, which may contain an analyte (e.g., polypeptide, polynucleotide, or fragment thereof) for which an analyte assay is desired. The sample may be a biological sample, such as a biological fluid or a biological tissue. In certain embodiments, a biological sample is a salivary sample. Such a sample may include diverse cells, proteins, and genetic material. Examples of biological tissues also include organs, tumors, lymph nodes, arteries and individual cell(s). Examples of biological fluids include urine, blood, plasma, serum, saliva, semen, stool, sputum, cerebral spinal fluid, tears, mucus, amniotic fluid or the like.

By the term “specifically binds,” as used herein with respect to an antigen binding molecule is meant an antigen binding molecule which recognizes a specific antigen, but does not substantially recognize or bind other molecules in a sample. For example, an antigen binding molecule that specifically binds to an antigen from one species may also bind to that antigen from one or more species. But, such cross-species reactivity does not itself alter the classification of an antigen binding molecule as specific. In another example, an antigen binding molecule that specifically binds to an antigen may also bind to different allelic forms of the antigen. However, such cross reactivity does not itself alter the classification of an antigen binding molecule as specific. In some instances, the terms “specific binding” or “specifically binding,” can be used in reference to the interaction of an antigen binding molecule, an antibody, a protein, or a peptide with a second chemical species, to mean that the interaction is dependent upon the presence of a particular structure (e.g., an antigenic determinant or epitope) on the chemical species; for example, an antigen binding molecule or an antibody recognizes and binds to a specific protein structure rather than to proteins generally. If an antigen binding molecule is specific for epitope “A”, the presence of a molecule containing epitope A (or free, unlabeled A), in a reaction containing labeled “A” and the antigen binding molecule, will reduce the amount of labeled A bound to the antigen binding molecule.

The term “subject” is intended to include living organisms in which an immune response can be elicited (e.g., mammals). A “subject” or “patient,” as used therein, may be a human or non-human mammal. Non-human mammals include, for example, non-human primates, livestock, and pets, such as simian, ovine, bovine, porcine, canine, feline, and murine mammals. Preferably, the subject is human.

A “target site” or “target sequence” refers to a genomic nucleic acid sequence that defines a portion of a nucleic acid to which a binding molecule may specifically bind under conditions sufficient for binding to occur.

The term “therapeutic” as used herein means a treatment and/or prophylaxis. A therapeutic effect is obtained by suppression, remission, or eradication of a disease state.

As used herein, the term “tissue” refers to a mammalian tissue, such as connective tissue, muscle tissue, nervous tissue, and epithelial tissue. Examples of connective tissue include bone, cartilage, blood, and fat. Examples of muscle tissue include skeletal muscle, smooth muscle, and cardiac muscle. Nervous tissue is found in the nervous system, including the brain and spinal cord. Epithelial tissue is found in the skin, the lining of the mouth and nose, and the lining of the digestive system. In certain embodiments, the tissue of the present invention is not skin. In some embodiments, the tissue is muscle (e.g., skeletal muscle). In some embodiments, the tissue is bone. In some embodiments, the tissue is cartilage.

As used herein, the term “transplantation” refers to the process of taking a cell, tissue, or organ, called a “transplant” or “graft” from one individual and placing it or them into a (usually) different individual. The individual who provides the transplant is called the “donor” and the individual who received the transplant is called the “host” (or “recipient”). An organ, or graft, transplanted between two genetically different individuals of the same species is called an “allograft”. A graft transplanted between individuals of different species is called a “xenograft”.

As used herein, “transplant rejection” refers to a functional and structural deterioration of the organ due to an active immune response expressed by the recipient, and independent of non-immunologic causes of organ dysfunction.

As used herein, the term “tolerance” is a state of immune unresponsiveness specific to a particular antigen or set of antigens induced by previous exposure to that antigen or set. Tolerance is generally accepted to be an active process and, in essence, a learning experience for T cells. Tolerance, as used herein, refers to the inhibition of a graft recipient's ability to mount an immune response which would otherwise occur, e.g., in response to the introduction of a non-self MHC antigen into the recipient. Tolerance can involve bumoral, cellular, or both humoral and cellular responses.

To “treat” a disease as the term is used herein, means to reduce the frequency or severity of at least one sign or symptom of a disease or disorder experienced by a subject. It will be appreciated that, although not precluded, treating a disorder or condition does not require that the disorder, condition or symptoms associated therewith be completely eliminated.

The term “xenogeneic” with reference to an organ, tissue, or cell denotes that the organ, tissue, or cell originates from an individual that is of a different species than the recipient.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The following abbreviations are used herein: ECM, extracellular matrix; HA, hyaluronic acid; H&E, hematoxylin and eosin; HUVECs, Human umbilical vein endothelial cell; KO, knock-out; SEM, scanning electron microscope; TSP2, thrombospondin-2: WT, wild-type.

### DESCRIPTION

The present invention relates to compositions and methods for promoting tissue regeneration in a subject in need thereof. In certain aspects, the subject is administered a decellularized ECM of a tissue, wherein the tissue lacks functional TSP2. In some embodiments, the tissue is from a mammal. In one embodiment, the mammal is a human.

In certain aspects, the invention provides a decellularized ECM of a tissue, wherein the tissue lacks TSP2. In some embodiments, the tissue is from a mammal. In one embodiment, the mammal is a human.

In some embodiments, the ECM is formulated as a hydrogel, such as a tunable hydrogel. In certain embodiments, the decellularized ECM is chemoattractant to at least one cell type selected from the group consisting of endothelial cell, mesenchymal cell, myocyte, fibroblast, and osteoblast. In some embodiments, the ECM and compositions of the invention are useful for the methods recited elsewhere herein.

### Methods of the Invention

### Method of Promoting Tissue Regeneration

In some aspects, the invention provides a method for promoting tissue regeneration in a subject in need thereof, the method comprising administering to the subject a decellularized ECM of a tissue, wherein the tissue lacks functional TSP2. In some embodiments, the tissue is from a mammal.

Promoting tissue regeneration in a subject includes, but is not necessarily limited to, promoting cellular migration, cellular invasion, vascular growth and maturation, and/or wound repair in the site of ECM administration. In some embodiments, promoting tissue regeneration comprises accelerating cellular migration, enhancing cellular invasion, enhancing vascular growth and maturation of a location to be treated, and enhancing wound repair in a subject in need thereof. In certain embodiments, cellular migration, cellular invasion, vascular growth and maturation, and/or wound repair are enhanced or accelerated at the site of ECM administration to the subject as compared to a location administered an ECM originating from a wild-type tissue, or an untreated location.

In certain embodiments, the tissue lacking functional TSP2 is selected from the group consisting of connective tissue, muscle tissue, nervous tissue, and epithelial tissue. Examples of connective tissue include bone, cartilage, blood, and fat. Examples of muscle tissue include skeletal muscle, smooth muscle, and cardiac muscle. Nervous tissue is found in the nervous system, including the brain and spinal cord. Epithelial tissue is found in the skin, the lining of the mouth and nose, and the lining of the digestive system. In certain embodiments, the tissue lacking functional TSP2 is not skin. In some embodiments, the tissue lacking functional TSP2 is muscle (e.g., skeletal muscle, smooth muscle, or cardiac muscle). In some embodiments, the tissue lacking functional TSP2 is skeletal muscle. In some embodiments, the tissue lacking functional TSP2 is bone.

In certain embodiments, the tissue lacking functional TSP2 is formulated as a hydrogel. In some embodiments, the ECM is dried and rehydrated in an aqueous solution to form the hydrogel. In some embodiments, the ECM is treated to form a pre-gel as described herein and the pre-gel is further treated to form the hydrogel as described herein.

The method for promoting tissue regeneration in a subject in need thereof is performed at one or more treatment site(s) of the subject. That is, in certain embodiments, the ECM or hydrogel is administered to one or more treatment site(s) of the subject. In some embodiments, the treatment site(s) may be any site in need of tissue regeneration, such as, but not limited to, a wound or injury. In other embodiments, the ECM or hydrogel is administered by any method known in the art, e.g., subcutaneous, intramuscular, intraosseous, or topical administration.

In certain embodiments, tissue lacking functional TSP2 is obtained by a method described herein. For example, cells of the tissue lacking functional TSP2 comprise one or both of (a) a TSP2 knockout allele, and (b) suppressed TSP2 gene expression.

In certain aspects, the method enhances at least one biological response at the treatment site, as compared to a site administered a decellularized ECM originating from a tissue comprising functional TSP2, or compared to an untreated site. In some embodiments, the biological response at the treatment site is selected from the group consisting of cellular migration towards the treatment site, cellular invasion of the treatment site, vascular growth and maturation, innervation, angiogenesis, and wound repair.

In certain embodiments, the tissue lacking functional TSP2 is muscle and the subject suffers from at least one condition selected from the group consisting of a muscle injury, a myopathy, a genetic myopathy, an inflammatory myopathy, an endocrine myopathy, a neuromuscular disorder, amyotrophic lateral sclerosis, Charcot-Marie-Tooth disease, multiple sclerosis, a muscular dystrophy, Duchenne muscular dystrophy, Becker muscular dystrophy, Limb Girdle muscular dystrophy, spinal muscular atrophy, Guillain-Barre Syndrome, Chronic Inflammatory Demyelinating Polyneuropathy, Multifocal Motor Neuropathy, Myasthenia Gravis, Pompe's Disease, Drop Head Syndrome (floppy head syndrome), multisystemic smooth muscle dysfunction syndrome, peripheral vascular disease, congenital heart disease, a cardiomyopathy, coronary artery disease, heart attack, heart valve disease, hypertension, hernia, type 1 diabetes, type 2 diabetes, alcohol use, and tobacco use.

In certain embodiments, the tissue lacking functional TSP is cartilage and the subject suffers from at least one condition selected from the group consisting of osteoarthritis, rheumatoid arthritis, avascular necrosis, costochondritis, a fracture, lupus, Maffucci syndrome, osteoporosis, achondroplasia, conclusion, and herniation.

In certain embodiments, the tissue lacking functional TSP2 is bone and the subject suffers from at least one condition selected from the group consisting of a bone injury, a bone disorder, osteoporosis, osteopetrosis, osteonecrosis, osteogenesis imperfecta, osteoarthritis, rheumatoid arthritis, type 1 diabetes, type 2 diabetes, lupus, celiac disease, hyperthyroidism, infection of bone or joint, Paget's Disease of Bone, fibrous dysplasia, tobacco use, and weight loss surgery.

In various aspects, the subject of the method is a mammal, such as, but not limited to, a human, a non-human primate, a pig, a sheep, or a mouse. In certain embodiments, the subject is a human. In one embodiment, the tissue lacking functional TSP2 originates from a mammal, such as, but not limited to, a mouse, a pig, a non-human primate, and a human. In other embodiments, the tissue lacking functional TSP2 is autologous, allogeneic, or xenogeneic relative to the subject. In the case of allogeneic or xenogeneic tissue, the tissue may be genetically engineered to be more immunologically compatible with the subject.

In certain embodiments, the decellularized ECM is formulated with at least one therapeutic agent. In some embodiments, the method for promoting tissue regeneration further comprises administering a therapeutic agent, or a pharmaceutical composition comprising a therapeutic agent, to the subject. Suitable therapeutic agents to be formulated with the decellularized ECM or administered separately from the decellularized ECM are known to those in the art and include, but are not limited to, an immunosuppressive agent, an anti-inflammatory agent, an antimetabolite, an antibiotic, an antibody, a growth factor, a cytokine, a gene therapy, an immunomodulator, and any combination thereof.

In other aspects, the invention provides a method for tissue formation, the method comprising seeding a decellularized ECM of a tissue with one or more types of cells, wherein the tissue lacks TSP2. In some embodiments, the decellularized ECM is a scaffold for seeding the one or more types of cells therein. In some embodiments, the method is performed ex vivo, in vivo, or a combination thereof. In one embodiment, the method further comprises implanting the decellularized ECM seeded with the one or more types of cells in a subject, wherein the one or more cells populate the decellularized ECM to form tissue in vivo. In another embodiment, the method further comprises providing a growth condition sufficient for the one or more cells to populate the decellularized ECM. In some embodiments, the growth condition comprises growth factors in amounts sufficient for the one or more types of cells to grow and/or proliferate.

In some aspects, the invention provides an engineered tissue comprising a decellularized ECM of a tissue lacking TSP2 and having one or more types of cells grown and/or proliferated therein.

In still further embodiments, the tissue (e.g., bone, cartilage, muscle) that lacks functional TSP2 can be a source of material that provides for a decellularized ECM prepared therefrom that lacks the functional TSP2. In other embodiments, hydrogels, scaffolds, and other materials lacking the functional TSP2 can be prepared from the decellularized ECM lacking the functional TSP2.

In some embodiments, the decellularized ECM lacking the functional TSP2, and the hydrogels, scaffolds, and other material prepared therefrom that also lack the functional TSP2, are capable of promoting re-endothelialization. In other embodiments, the decellularized ECM lacking the functional TSP2, and the hydrogels, scaffolds, and other material prepared therefrom that also lack the functional TSP2, are capable of promoting re-endothelialization by the recipient's vascular endothelial cells post-transplantation or implantation, or by autologous or allogeneic vascular endothelial cells seeded onto and/or therein prior to transplantation or implantation. In some embodiments, vascular endothelial cells which grow onto and/or therein the decellularized ECM lacking the functional TSP2, and the hydrogels, scaffolds, and other material prepared therefrom that also lack the functional TSP2, provide an interface between the vascular endothelial cells and the recipient's immune surveillance mechanisms, for the purpose of avoiding thrombosis and preventing or minimizing rejection. In other embodiments, the decellularized ECM lacking the functional TSP2, and the hydrogels, scaffolds, and other material prepared therefrom that also lack the functional TSP2, are less adhesive for blood glycoprotein von Willebrand Factor (vWF) as compared to a reference wild-type decellularized ECM having functional TSP2, and the hydrogels, scaffolds, and other material prepared therefrom having TSP2. In some embodiments, the decellularized ECM lacking the functional TSP2, and the hydrogels, scaffolds, and other material prepared therefrom that also lack the functional TSP2, support vascular endothelial cell colonization by promoting adhesion, regulating growth factor activity, and/or modulating protease activity, and/or by directly activating intracellular second messenger systems. In some embodiments, the decellularized ECM lacking the functional TSP2, and the hydrogels, scaffolds, and other material prepared therefrom that also lack the functional TSP2, are: (a) non-thrombogenic; (b) pro-angiogenic, pro-migratory (i.e. supportive of, and efficiently promoting re-endothelialization); and/or (c) nonimmunogenic (with respect to a recipient).

### Methods of Preparing Decellularized ECM

In other aspects, the present invention relates to methods of preparing a decellularized ECM of a tissue, wherein the tissue lacks TSP2. In some embodiments, the tissue is from a mammal.

In some embodiments, the ECM is obtained from a mammalian tissue lacking functional TSP2. In one embodiment, the tissue is obtained from a mammal using methods known to those skilled in the art, such as cutting, excision, and/or shaving. In some embodiments, tissue can be derived from aggregates of cells, an organ, portions of an organ, or combinations of organs. In some instances, the tissue is isolated from a mammal, for example and without limitation, human, non-human primate, monkey, pig, cattle, sheep, and mouse. In some embodiments, the tissue is isolated from any tissue of a subject (e.g., mammal), for example and without limitation, muscle (e.g., skeletal muscle, connective, myocardium), bone, urinary bladder, liver, central nervous system (CNS), adipose tissue, small intestine, large intestine, colon, esophagus, pancreas, vascular tissue (e.g., artery, vein) and heart.

The ECM is the natural substrate on which cells migrate, proliferate, and differentiate. These components are linked in such a way that the resulting structure is tri-dimensional scaffolding in vivo. Thus, in some embodiments, the decellularized ECM of the invention provides scaffolding, support and strength to cells grown on and/or therein, allowing those cells to differentiate and mediate physiologic responses. ECMs from different anatomic sites may vary in their ability to support and allow for proper differentiation of cells not from that respective anatomic site. In some embodiments, without wishing to be bound by any theory, the decellularized ECM is of tissues derived from the same species as the recipient or from species known to in the art to have compatibility with the recipient. In some embodiments, if the recipient is a human, the decellularized ECM originates from a tissue of an animal known to have compatibilities with humans such as, but not limited to, a primate or a pig. In some embodiments, the animal has been genetically engineered, such as a genetically engineered pig or primate, to be more compatible with the human recipient compared to the wild-type tissue.

### Tissue Lacking Functional TSP2

Generation of a tissue lacking functional TSP2 can be accomplished in a number of ways. In some aspects, the absence of a functional TSP2 in the tissue may be achieved by a full or partial knock-out of the TSP2 gene in the tissue. Methods of gene knock-out are well known in the art. In some embodiments, knock-out can be accomplished through a variety of well-established molecular techniques. In some embodiments, individual stem cells are genetically transfected with the DNA construct for the goal of creating a transgenic organism (e.g., mammal) that has the altered gene. Embryonic stem cells are genetically transformed and inserted into early embryos. The resulting transgenic animals with the genetic alteration in their germline cells then pass the knock-out to future generations. For instance, a knock-out mouse refers to a mouse in which a gene or genes have been mutated such that the activity of the gene has been reduced or eliminated. In some embodiments, the TSP2 gene is knocked out in a genetically engineered organism such as a mouse or a pig. In other aspects, the TSP2 gene is knocked down in a tissue using molecular techniques known in the art such as, but not limited to, RNA interference (RNAi), small hairpin RNA (shRNA) and Clustered Regularly Interspaced Short Palindromic Repeats (CRISPRs). Knocked-down expression of TSP2 is useful for generation of tissue lacking functional TSP2 in organisms where knock-out of TSP2 is not usually possible or desired, e.g., in humans. In some embodiments, the tissue lacking functional TSP2 comprises a TSP2 knockout genotype. In other embodiments, expression of TSP2 in the tissue has been knocked down. In one embodiment, expression of TSP2 is diminished when compared with wild type expression, and/or is eliminated altogether. In some aspects, the characteristics of an ECM produced from a TSP2 knock-down are optimized and similar to those of an BCM produced from a TSP2 knock-out.

In some embodiments, cells of the tissue lacking functional TSP2 comprise one or both of the following: (a) a TSP2-null knockout allele; and (b) suppressed TSP2 gene expression. In certain embodiments, the TSP2 null knockout allele or suppressed TSP2 gene expression is obtained via a genetic engineering technique comprising a nuclease selected from the group consisting of a clustered regularly interspaced short palindromic repeats (CRISPR)-associated (CAS) nuclease, a transcription activator-like effector nuclease (TALEN), and a zinc-finger nuclease. In some embodiments, cells of the tissue lacking functional TSP2 comprise an inhibitory RNA molecule which suppresses TSP2 gene expression. Inhibitory RNA molecules are well-known in the art and include, but are not limited to, an RNA interference (RNAi) RNA, a short hairpin RNA (shRNA), a small interfering RNA (siRNA), a trans-acting siRNA (tasiRNA), a micro RNA (miRNA), an antisense RNA (asRNA), a long noncoding RNA (lncRNA), a CRISPR RNA (crRNA), a trans-activating crRNA (tracrRNA), a guide RNA (gRNA), a single guide RNA (sgRNA), a double-stranded RNA (dsRNA), a ribozyme, and any combination thereof.

In some embodiments, the tissue is a muscle, a cartilage, a connective tissue, a tendon, a ligament, or a bone. In some embodiments, the tissue lacking functional TSP2 is muscle, such as skeletal muscle, smooth muscle, or cardiac muscle. In some embodiments, the tissue lacking functional TSP2 is bone. In some embodiments, the tissue lacking functional TSP2 is cartilage. In certain embodiments, the tissue lacking functional TSP2 is not skin.

### Decellularizing Tissue

The tissue of this invention can be decellularized by methods known in the art. In one aspect, decellularization of the tissue is performed to prevent a pro-inflammatory response in the subject. As such, in one aspect, a decellularized ECM refers to ECM material that is decellularized to the extent that a pro-inflammatory response, and thus growth of fibrotic tissue, is not elicited to any substantial degree in favor of constructive remodeling.

In certain embodiments, decellularization of the tissue comprises treating the tissue with a decellularization solution comprising one or more of trypsin-EDTA, TRIS, triton, sodium deoxycholate (SDC), and the like. In certain embodiments, the decellularization solution does not comprise sodium dodecyl sulfate (SDS).

In certain embodiments, the decellularized ECM as described herein retains activity of at least a portion of its structural and non-structural biomolecules, including, but not limited to, collagens, elastins, laminins, glycosaminoglycans, proteoglycans, antimicrobials, chemoattractants, cytokines, and/or growth factors. In certain embodiments, the activity of the biomolecules within the ECM can be removed chemically or mechanically, for example, by cross-linking and/or by dialyzing the ECM. In one aspect, the decellularized ECM composition of this invention is cross-linked by addition of a chemical cross-linking agent. In other aspects, the ECM materials described herein essentially have not been cross-linked and/or dialyzed. Thus, in one aspect, the ECM material is not cross-linked and/or dialyzed in anything but a trivial manner which does not substantially affect the gelation and functional characteristics of the ECM material in its uses described herein.

In certain embodiments, the tissue lacking functional TSP2 is muscle. In some embodiments, decellularizing the muscle tissue comprises treating the muscle at room temperature (RT) sequentially with (i) an aqueous trypsin-EDTA solution for about 5-7 hours; (ii) an aqueous H₂O₂ solution for about 15 minutes; (iii) an aqueous Triton X-100/EDTA/Tris solution for about 5-7 hours; (iv) a fresh aqueous Triton X-100/EDTA/Tris solution for about 5-15 hours; and (v) an aqueous Triton X-100/sodium deoxycholate (SDC) solution for about 5-7 hours. Exemplary concentrations for the decellularization solutions include: (i) 0.25% trypsin-EDTA solution; (ii) 3% H₂O₂ solution; (iii) 1% Triton X-100/0.26% EDTA/0.69% Tris; (iv) 1% Triton X-100/0.26% EDTA/0.69% Tris; and (v) 1% Triton X-100/0.2% SDC.

In certain embodiments, the tissue lacking functional TSP2 is cartilage. Generally, prior to decellularization, the sample has skin and fat remnants removed. The sample is then sterilized with ethanol, and oxidized with 3% hydrogen peroxide. Decellularlization comprises mixing the sample with 1% Triton solution/EDTA/Tris for about 36 hours. After, the sample is washed with ethanol/peracetic acid/ddH2O. Finally, the sample is incubated in FBS free culture media with 1% antibiotics overnight and then lyophilized.

In certain embodiments, the tissue lacking functional TSP2 is bone. Generally, the bone is sectioned to produce fragments. The bone is then demineralized to generate demineralized bone matrix (DBM). Bone demineralization can be achieved using any method known in the art. For example, in some embodiments, the bone is demineralized under agitation, e.g., at about 300 rpm, in an aqueous acid solution at about RT for about 24 hours (i.e., about 20-28 hours). In some embodiments, the aqueous acid solution comprises about 0.5 N HCl (approximately 25 ml/g bone). For embodiments where the tissue is bone, decellularizing the tissue comprises decellularizing the DBM. In some embodiments, decellularizing the DBM comprises incubating the DBM with agitation with an aqueous trypsin-EDTA solution at approximately 37° C. for about 24 hours (i.e., about 20-28 hours). Exemplary concentrations for the trypsin-EDTA solution include 0.05% trypsin and 0.02% EDTA.

### Formulation of the ECM

In one embodiment, the decellularized ECM of this invention is formulated as sheet of material such as but not limited to STRATTICE™ or ALLODERM™ regenerative tissue matrix (Allergan, Dublin, Ireland).

In certain embodiments, the decellularized ECM is formulated as a hydrogel. In some embodiments, the tissue is muscle and the ECM is dried, such as by lyophilization, and then rehydrated to generate the hydrogel. Suitable solutions for rehydrating the ECM include, but are not limited to, water, saline, phosphate-buffered saline.

In some embodiments, the tissue is bone, and the decellularized ECM is formed into a pre-gel by removing residual cellular materials and digesting with pepsin for about 96 hours (e.g., for about 84 to about 108 hours) at RT. Next, the pepsin digestion is stopped and the digested ECM pre-gel is incubated for about 1 hour at higher temperature, such as at approximately 37° C., whereby the pre-gel forms into a hydrogel.

In some instances, such as when the tissue is bone, the hydrogel is produced from a reverse gel (i.e., a pre-gel), which forms a hydrogel upon an increase in temperature. As the temperature rises above a certain temperature in a reverse gel, a hydrogel is formed. The general concept of reverse gelation of polymers and, e.g., its relation to lower critical solution temperature (LCST) are broadly known in the chemical arts. The ECM compositions described herein are prepared, for example, from decellularized or devitalized, intact ECM as described elsewhere herein. An ECM gel is prepared by digestion of the ECM material with an acid protease, neutralization of the material to form a pre-gel, and then raising the temperature of the pre-gel above a gelation temperature, for example the LCST of the pre-gel, to cause the pre-gel to become a gel. As used herein, the term “gel” includes hydrogels. The transition temperature for acid-protease-digested from solution to gel is typically within the range of from about 10° C. to 40° C. and any increments or ranges therebetween, for example from about 20° C. to 35° C. For example, the pre-gel can be warmed to about 37° C. to form a hydrogel.

In some instances, such as when the tissue is cartilage, the decellularlized ECM is formed into a hydrogel by weighing lyophilized tissues and then the tissues undergo Cryomill™ or cryogenic grinding prior to digestion.

Tissue for preparation of ECM, ECM-derived pre-gel solutions, and gels as described herein may be harvested in any useful manner. Decellularized or devitalized ECM can be dried, either lyophilized (freeze-dried) or air dried. The ECM composition is optionally comminuted at some point, for example prior to acid protease digestion in preparation of an ECM gel, for example prior to or after drying. In certain embodiments, the decellularized TSP2-null ECM of the invention is comminuted. The comminuted ECM can also be further processed into a powdered form by methods, for example and without limitation, such as grinding or milling in a frozen or freeze-dried state.

In order to prepare solubilized ECM tissue, the ECM is digested with an acid protease in an acidic solution to form a digest solution. As used herein, the term “acid protease” refers to an enzyme that cleaves peptide bonds, wherein the enzyme has increased activity of cleaving peptide bonds in an acidic pH. For example and without limitation, acid proteases include pepsin and trypsin and mixtures thereof.

As an example, the digest solution of ECM is kept at a constant stir for a certain amount of time at room temperature. In one aspect, the pH is maintained at less than pH 4.0 or at pH 2.0=0.3 during acid protease digestion of the decellularized tissue as described herein. The ECM digest can be used immediately or can be stored at −20° C. or frozen at, for example and without limitation, −20° C. or −80° C. In certain aspects, the ECM digest is snap frozen in liquid nitrogen. To form a “pre-gel” solution, the pH of the digest solution is raised to a pH between 6.8 and 7.8. The pH can be raised by adding one or more of a base or an isotonic buffered solution, for example and without limitation, NaOH or PBS at pH 7.4. In some aspect, the pre-gel solution is freeze dried and stored at −20° C. or −80° C. until needed. The method optionally does not include a dialysis step prior to gelation, yielding a more complete ECM-like matrix that typically gels at 37° C. more slowly than comparable collagen or dialyzed ECM preparations. The gel therefore retains more of the qualities of native ECM due to retention of many native soluble factors, such as, without limitation, cytokines. Without intending to be limited to any particular theory, these factors contribute to chemoattraction of cells and proper rearrangement of tissue at the site of wound or injury, rather than a fibrotic response that leads to unwanted scarring. In other embodiments, the ECM is dialyzed prior to gelation to remove certain soluble components.

As used herein, the term “isotonic buffered solution” refers to a solution that is buffered to a pH between 6.8 and 7.8, e.g., pH 7.4, and that has a balanced concentration of salts to promote an isotonic environment. As used herein, the term “base” refers to any compound or a solution of a compound with a pH greater than 7. For example and without limitation, the base is an alkaline hydroxide or an aqueous solution of an alkaline hydroxide. In certain embodiments, the base is NaOH, or NaOH in PBS. This “pre-gel” solution can, at that point be incubated at a suitably warm temperature, for example and without limitation, at about 37° C. to gel.

In the method of preparing an ECM gel, the ECM may be partially or completely digested with the acid protease, such as pepsin. The digested ECM is then neutralized to a pH of 6.8-7.8, e.g., 7.2-7.6, or 7.4 and the neutralized and digested ECM material is gelled by incubation at a temperature at which the material gels, e.g., at a temperature above 20, 25, 30, or 35° C., such as at 37°. The degree of digestion can be determined by comparison on a gel, or by ascertaining the degree of degradation of hyaluronic acid, for example by Western blot (anti-hyaluronic acid antibodies are commercially-available from multiple sources) or chromatographic methods, as are broadly known. For example in a partial digestion, hyaluronic acid is digested less than 50%, 40%, 30%, 25%, 20% or 10%.

Compositions and methods of the present invention are useful for treatment of mammals, and particularly humans. In certain embodiments, the mammal is immune compromised, suffers from an autoimmune disease, has or will have transplant, or suffers from a condition with high risk for wounds. In certain embodiments, the mammal suffers from at least one condition selected from the group consisting of: diabetes, hernia, mastectomy, peripheral vascular disease, and neuropathy. In certain embodiments, the mammal is in need for regenerative medicine to replace or repair a tissue or organ that has been damaged by a disease, a trauma or a congenital issue (such as, but not limited to, empty nose syndrome). In other embodiments, the compositions and methods of the present invention are useful for aesthetic purposes.

### Combination Therapies

The decellularized ECM described herein is also useful when combined with at least one additional compound. The additional compound may comprise commercially available compounds known to treat, prevent, or reduce the symptoms associated with graft transplants or implantation of a device into a subject.

In one aspect, the present invention contemplates that the decellularized ECM of the invention may be used in combination with a therapeutic agent such as an immunosuppressive agent. Non-limiting examples of immunosuppressive agents known in the art are cyclosporine, azathioprine, everolimus and glucocorticoids, mycophenolic acid, fingolimod, antimetabolites (such as, but not limited to, methotrexate, fluorouracil), antibiotics (such as, but not limited to, dactinomycin, mitomycin C, bleomycin), and antibodies (such as, but not limited to, Atgam, Muromonab-CD3, basiliximab, daclizumab).

In another aspect, the present invention provides the decellularized ECM of the invention as a delivery vehicle for one or more active pharmaceutical agents or drugs. In certain embodiments, the decellularized ECM of the invention further comprises at least one active pharmaceutical agent selected from the group consisting of a Rac1 inhibitor, a NFKB inhibitor, a p38 MAPK inhibitor, a RhoA inhibitor, a growth factor (including, but not limited to, VEGF, PDGF and BMP-2), Fasudil, Ripasudil, antibiotics, immune modulators (including, but not limited to, IL-4, IL-33 and IL-10), an anti-inflammatory (including, but not limited to, glucocorticoids and NSAIDs), a cytokine and oligonucleotides (including, but not limited to, siRNA, shRNA, plasmid DNA, and/or virus for gene therapy). In yet other embodiments, the at least one active pharmaceutical agent is an anti-inflammatory drug that influences the response of macrophages, such as, but not limited to BAY-11. In yet other embodiments, the at least one pharmaceutical agent is selected from the group consisting of CAS 1177865-17-6, SB 202190 SB203580, RKI-1447, and Y-27632.

### Pharmaceutical Compositions and Formulations

The invention includes the use of a pharmaceutical composition combined with the decellularized ECM preparation as described herein for use in the methods of the invention. The invention also includes the use of a pharmaceutical composition combined with the hydrogel formulations as described herein for use in the methods of the invention. In some embodiments, the method for promoting tissue regeneration further comprises administering a therapeutic agent, or a pharmaceutical composition comprising a therapeutic agent, to the subject, the method for promoting tissue regeneration further comprises administering a therapeutic agent, or a pharmaceutical composition comprising a therapeutic agent, to the subject. Suitable therapeutic agents to be formulated with the ECM or administered separately from the ECM are known to those in the art and include, but are not limited to, an immunosuppressive agent, an anti-inflammatory agent, an antimetabolite, an antibiotic, an antibody, a growth factor, a cytokine, a gene therapy, an immunomodulator, and any combination thereof.

Such a pharmaceutical composition is in a form suitable for administration to a subject, or the pharmaceutical composition may further comprise one or more pharmaceutically acceptable carriers, one or more additional ingredients, or some combination of these. The various components of the pharmaceutical composition may be present in the form of a physiologically acceptable salt, such as in combination with a physiologically acceptable cation or anion, as is well known in the art.

In an embodiment, the pharmaceutical compositions useful for practicing the method of the invention may be administered to deliver a dose of between 1 ng/kg/day and 100 mg/kg/day. In another embodiment, the pharmaceutical compositions useful for practicing the invention may be administered to deliver a dose of between 1 ng/kg/day and 500 mg/kg/day.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for inhalational, oral, rectal, vaginal, parenteral, topical, transdermal, pulmonary, intranasal, buccal, ophthalmic, intrathecal, intravenous or another route of administration. Other contemplated formulations include projected nanoparticles, liposomal preparations, resealed erythrocytes containing the active ingredient, and immunologically-based formulations. The route(s) of administration is readily apparent to the skilled artisan and depends upon any number of factors including the type and severity of the disease or wound being treated, the type and age of the veterinary or human patient being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it is understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

In certain embodiments, the compositions are formulated using one or more pharmaceutically acceptable excipients or carriers. Pharmaceutically acceptable carriers, which are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences, 1991, Mack Publication Co., New Jersey.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

The composition preferably includes an antioxidant and a chelating agent which inhibits the degradation of the compound. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition which may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

### Kit

In one aspect of the invention, a commercial kit is provided comprising the decellularized ECM or composition comprising the decellularized ECM described herein. A kit comprises suitable packaging material and the ECM or composition. In certain embodiments, the kit comprises a decellularized ECM formulated as a sheet of material (e.g. STRATTICE™ or ALLODERM™) or a digested decellularized ECM solution in a vessel, which may be the packaging, or which may be contained within packaging. In one embodiment, if the sheet of material or digest solution is neutralized, it may be frozen, cooled; e.g., kept at near-freezing temperatures, such as, without limitation, below about 4° C. or kept at room temperature, e.g., 20-25° C. In another embodiment, the kit comprises a first vessel containing an acidic solution comprising a pre-neutralization digest as described elsewhere herein, and a second vessel comprising a neutralizing solution comprising a base and/or buffer(s) to bring the acidic solution of the first vessel to physiological ionic strength and pH, to form a neutralized digest. In a further embodiment, the first vessel contains a terminally sterilized, lyophilized, pre-neutralization digest that can be hydrated using water or a suitable aqueous solution that optionally neutralizes the acid. In this embodiment, a second vessel is optionally provided comprising a neutralization solution as described above that is capable of both hydrating the lyophilized product and neutralizing it, or optionally a third vessel comprising water or any other suitable solution useful in hydrating the lyophilized product prior to neutralization with the neutralization solution. This kit also optionally comprises a mixing needle and/or a cold-pack. The vessel may be a vial, syringe, tube or any other container suitable for storage and transfer in commercial distribution routes of the kit.

### Administration/Dosing

The regimen of administration may affect what constitutes an effective amount. Several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

The compositions described herein find use as, without limitation, an injectable graft (e.g., xenogeneic, allogeneic or autologous) for tissues, for example, bone or soft tissues, in need of repair or augmentation most typically to correct a wound, a trauma or disease-induced tissue defects. The compositions also may be used as a filler for implant constructs comprising, for example, a molded construct formed into a desired shape for use in cosmetic or trauma-treating surgical procedures.

Administration of the compositions of the present invention to a subject (being a patient), preferably a mammal, more preferably a human, may be carried out using known procedures, at dosages and for periods of time effective to treat the patient. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 0.01 and 50 mg/kg of body weight/per day. One of ordinary skill in the art would be able to study the relevant factors and make the determination regarding the effective amount of the therapeutic compound without undue experimentation.

The compound can be administered to an animal as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon any number of factors, such as, but not limited to, the type and severity of the disease being treated, and the type and age of the animal. Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient. A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of wound in a patient.

### Routes of Administration

One skilled in the art will recognize that although more than one route can be used for administration, a particular route can provide a more immediate and more effective reaction than another route.

Routes of administration of any of the compositions of the invention include parenteral, sublingual, transdermal, transmucosal (e.g., sublingual, lingual, (trans) buccal, (trans) urethral, vaginal (e.g., trans- and perivaginally), (intra) nasal, and (trans) rectal), intravesical, intrapulmonary, intraduodenal, intragastrical, intrathecal, subcutaneous, intramuscular, intraosseous, intradermal, intra-arterial, intravenous, intrabronchial, inhalation, and topical administration. Suitable compositions and dosage forms include, for example, sheets of material (e.g. STRATTICE™ or ALLODERM™) or hydrogels that could be sutured into wounds or used as supporting meshes/slings, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

Particularly, the decellularized ECM formulation, such as a sheet of material (e.g. STRATTICE™ or ALLODERM™) or a hydrogel, may be implanted into a patient, human or animal, by a number of known methods. In certain embodiments, the ECM or ECM formulation is injected as a liquid into a desired site in the patient. As used herein, the term “seed,” “seeding,” or “seeded” refers to the addition, incorporation, propagation of, or spreading of a defined volume of a cell suspension or a defined cell number into a specific composition. The composition may be pre-seeded with cells, and then preferably injected using a larger-bore, e.g. 16 gauge needle, to prevent shearing of cells. In another embodiment, the composition is gelled within a mold (e.g. a silicone mold) or formulated as a hydrogel, and the gelled, molded product or the hydrogel product is then implanted into the patient at a desired site. The gelled, molded product may be pre-seeded (laid onto the molded gel or mixed in during gelation) with cells, such as cells of the patient.

In certain embodiments, the administration of the decellularized ECM or ECM hydrogel is at least one selected from the group consisting of subcutaneous, intramuscular, intraosseous, and topical. In other embodiments, the composition of the invention is injected, seeded or surgically implanted to the region to be treated.

In certain embodiments, the composition of the invention is applied to a bandage or dressing, which is then applied to the wound or treatment site of a subject. For example, in one embodiment, a dressing is soaked in a liquid solution or liquid suspension comprising decellularized ECM or ECM hydrogel. In another embodiment, an ointment comprising decellularized ECM is applied to a surface of a dressing or bandage. In yet other embodiments, the decellularized ECM or ECM hydrogel is incorporated into a pharmaceutical formulation including topical ointments, creams, aerosol sprays, and the like.

In certain embodiments, the administration route is a continuous subcutaneous administration for at least 2 days. In another embodiment, the administration route is a continuous subcutaneous administration for at least 20 days. In yet another embodiment, the administration route is a continuous subcutaneous administration for at least 30 days.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1: Preparation and Characterization of ECM Hydrogels Originating from Skeletal Muscle

### Materials and Methods

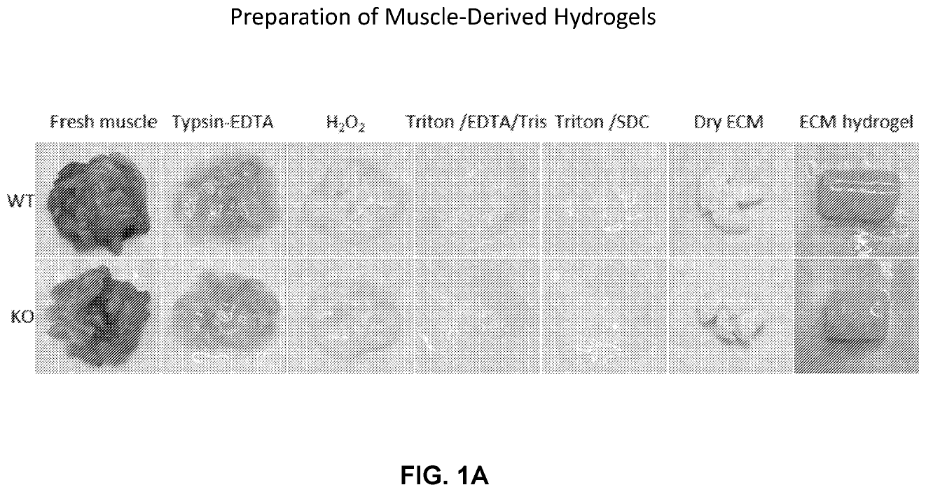

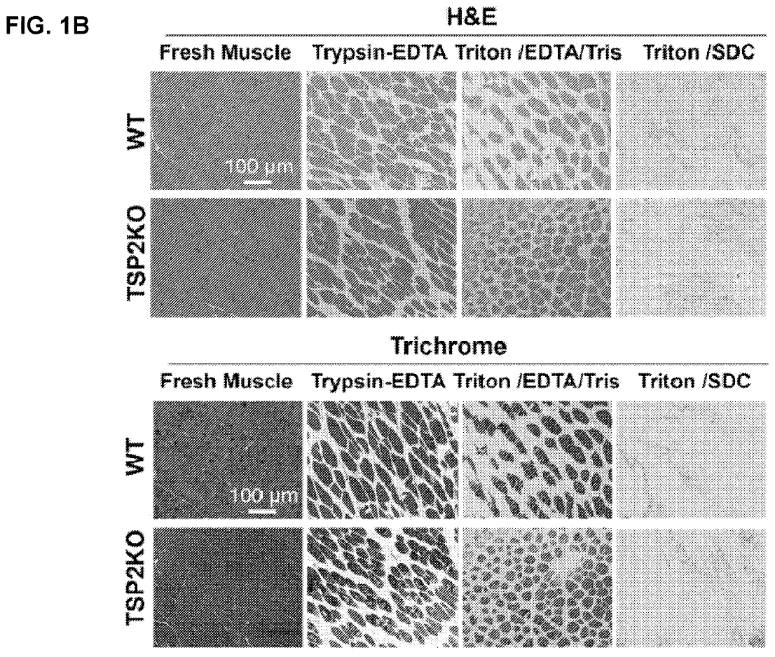

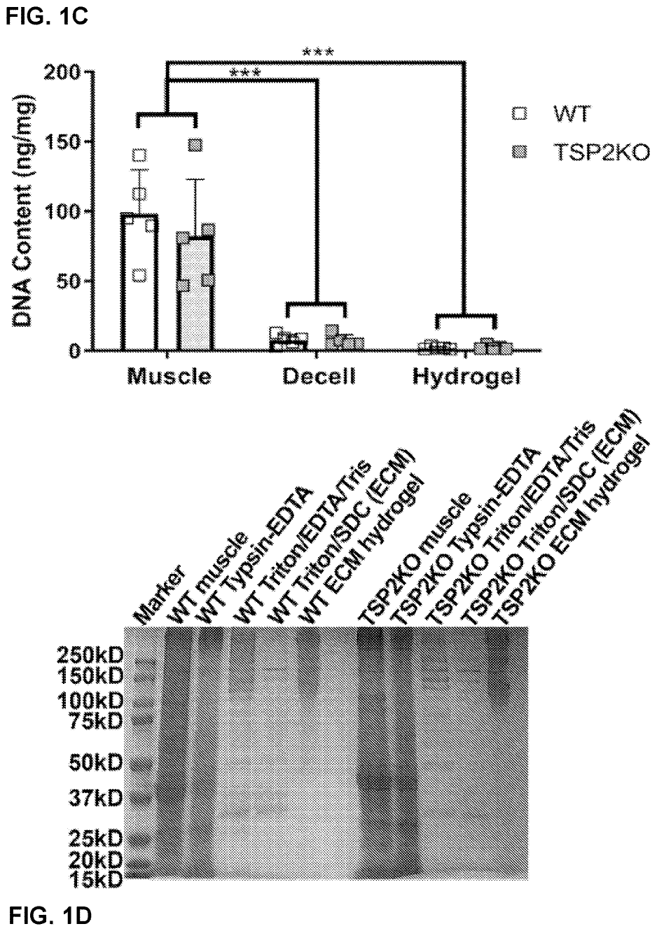

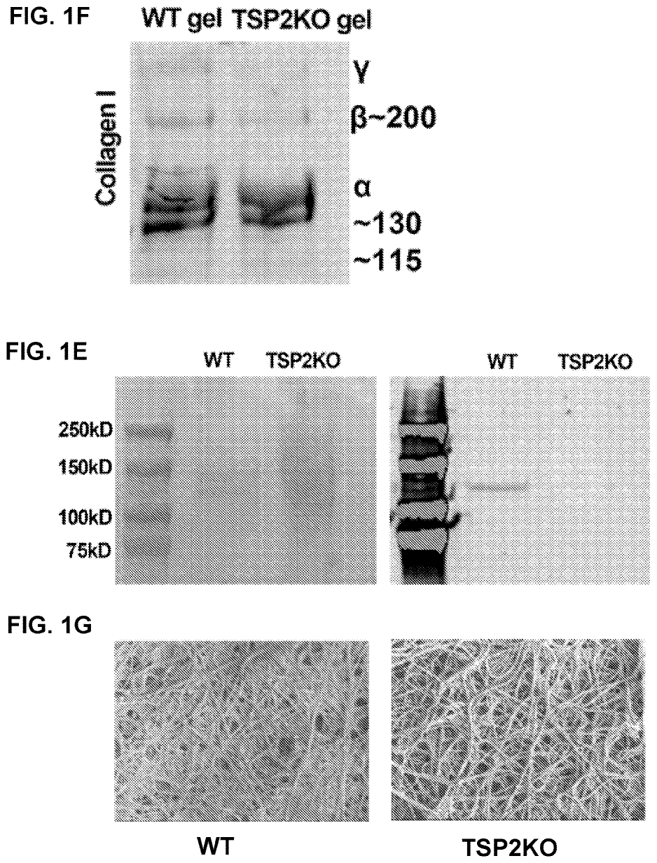

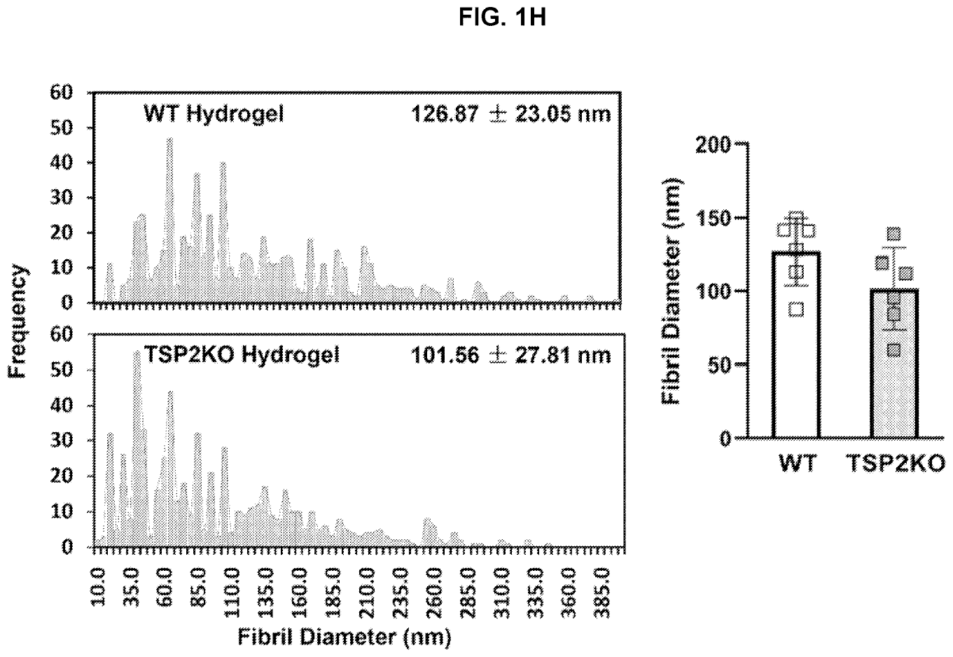

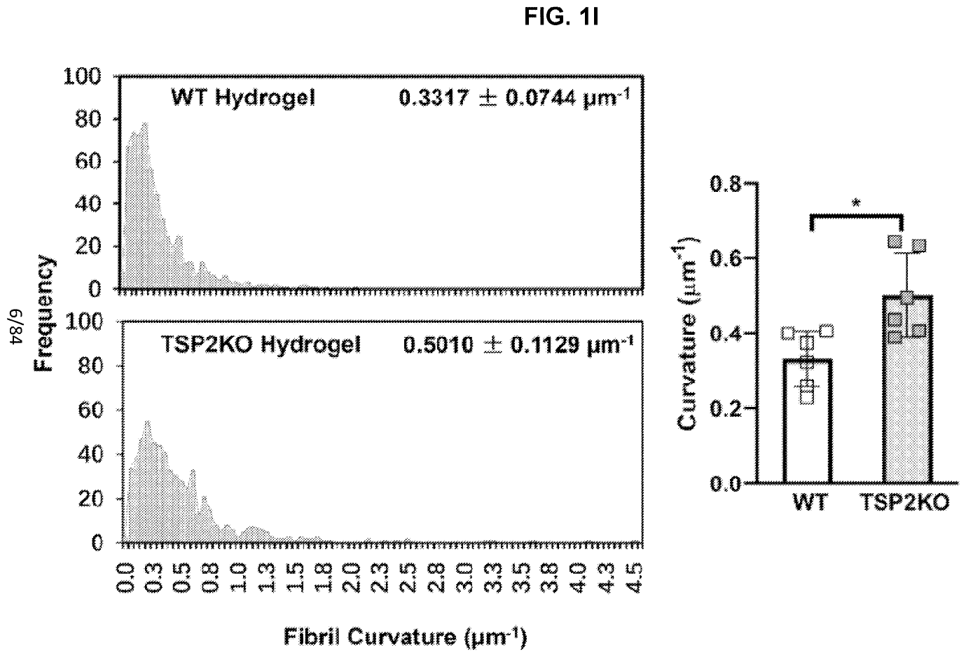

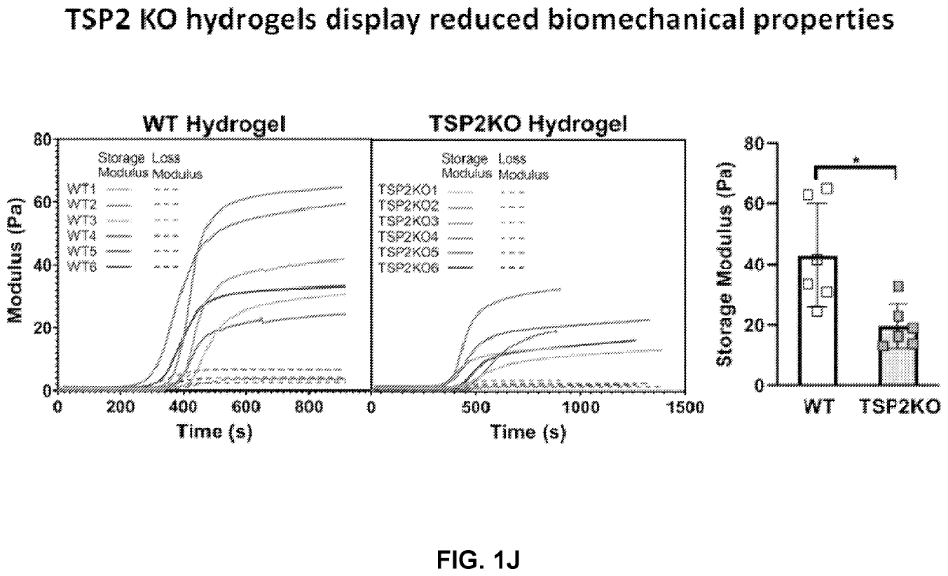

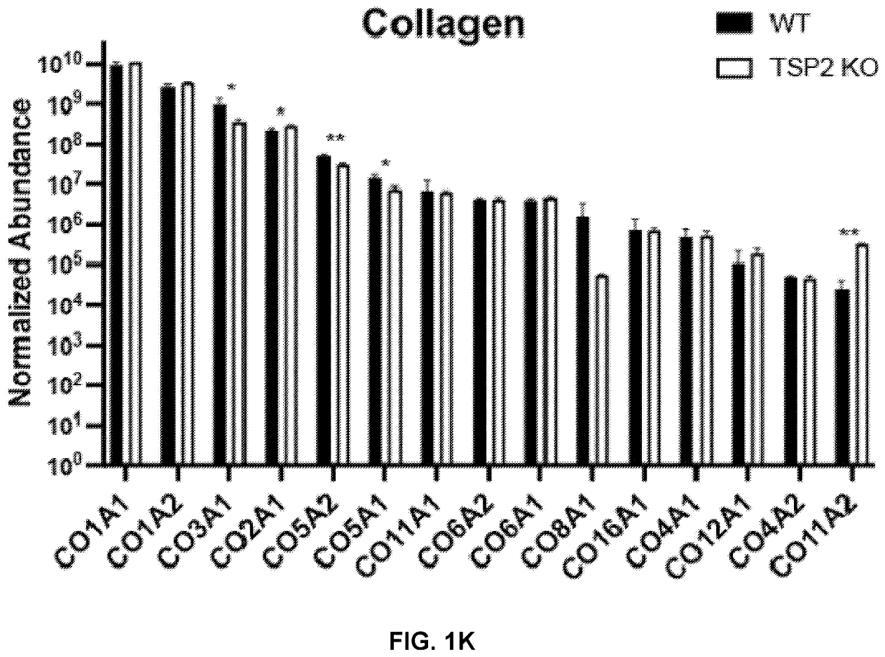

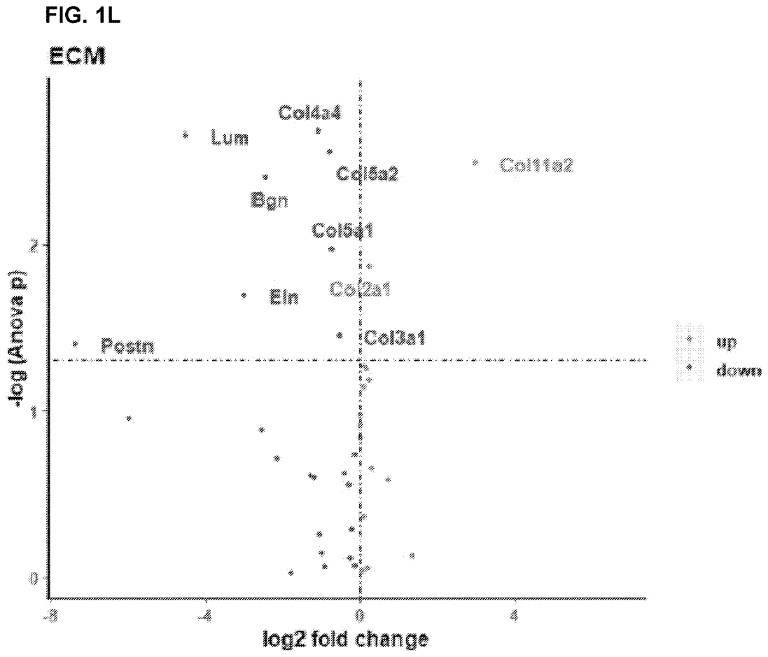

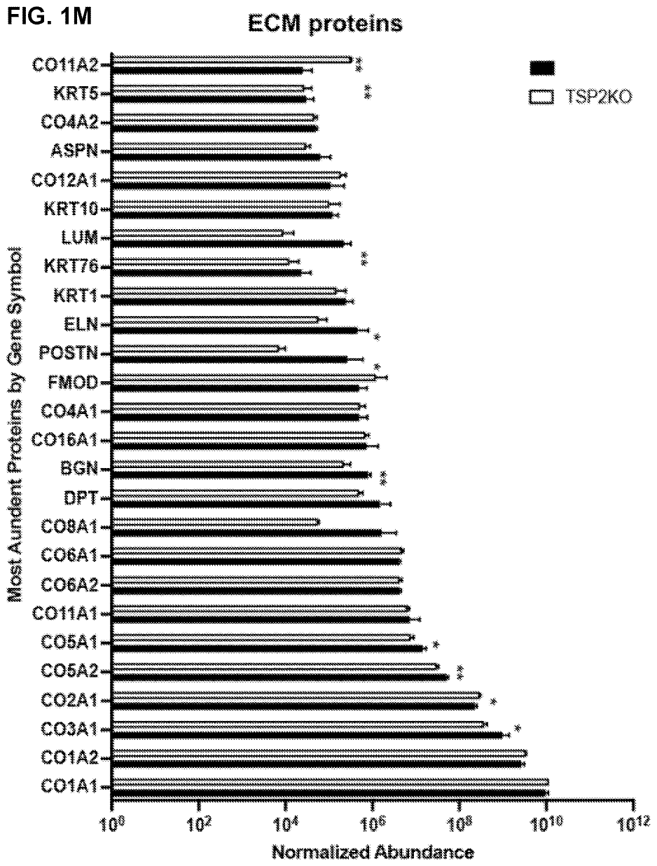

Isolation and Decellularization of Muscle. All procedures involving animal use were approved by the Animal Care and Use Committee of Yale University and abided by the regulations adopted by the National Institutes of Health. 12-14-week-old mice on a C57BL/6J background or TSP2-null (“TSP2KO”) mice were executed by inhalation of overdose CO₂ and muscles were subsequently harvested. Different from published approaches using SDS in the decellularization process, a gentler method was used to decellularize to maximize the retention of protein composition in the ECM. Firstly, instead of SDS, trypsin-EDTA, tris, triton, SDC, and the like were used. (FIG. 1A). Secondly, the working concentration of these agents were adjusted to the minimum limit (FIG. 1A). Meanwhile, to balance the effective decellularization with the protection of various proteins in ECM, a ‘cocktail decellularization method’ was used in this study. Specifically, various decellularization reagents were arranged and combined in the decellularization process (FIG. 1A). After using this method, the various proteins remained in the ECM (FIG. 1D).

After the muscles were washed with ddH₂O, they were incubated for 6 h in 0.25% Trypsin-EDTA (Gibco), followed by washes in ddH₂O three times for 15 min. Muscles were incubated in 70% ethanol for 12 h and 3% H₂O₂ (J. T. Baker) for 15 min, followed by two 15 min washes in ddH₂O. They were then incubated in 1% Triton X-100 (American Bioanalytical) in 0.26% Tris (American Bioanalytical)/0.69% EDTA (Sigma) for 6 h and then overnight. They were incubated for 6 h in 1% Triton X-100/0.2% Sodium deoxycholate (SDC, Sigma) and terminally sterilized in 0.1% peracetic acid (Sigma) in 4% ethanol for 2 h. Finally, they were washed in ddH₂O six times for 15 min each. All the above steps were performed at room temperature on an orbital shaker. Afterward, decellularized tissues were rinsed with ddH₂O, lyophilized, and stored at −80° C. until use. Tissues from each step were fixed with z-fix (Thermo Fisher Scientific) and stained with hematoxylin and eosin (H&E) and Trichrome staining.

Hydrogel Preparation. ECM derived from WT or TSP2KO muscle was digested in a solution of 1 mg/mL pepsin (Sigma) in 0.01 N HCl (J. T. Baker) for 72 h (10 mg dry weight ECM per milliliter of pepsin solution). The solubilized ECM was neutralized and buffered with sodium hydroxide ( 1/10 digest volume) and 10× phosphate-buffered saline (PBS) ( 1/9 digest volume). To prepare ECM-derived hydrogels, buffered and solubilized ECM was diluted to a final concentration of 10 mg/mL with PBS to form the pre-gel solution and stored on ice until use.

Quantitative Detection of DNA and Protein Content. The DNA of the fresh muscle, ECM and hydrogel derived from muscle was extracted using a DNeasy® Kit (QIAGEN), respectively. Briefly, samples were digested in lysis buffer T1 and proteinase-K at 60° C. for 24 h, and DNA was isolated according to kit instructions. NanoDrop (Thermo Scientific) readings were conducted on eluted DNA to confirm a DNA concentration of zero. The muscle, ECM and hydrogel were analyzed by SDS-poly-acrylamide gel electrophoresis (SDS-PAGE) to assess protein content. The 20 μl of each sample was added to each lane and compared against the Precision Plus Protein Dual Color Ladder (Bio-Rad). Gels were stained with Coomassie blue and imaged with an imaging scanner (Licor Odyssey). TSP2 and Collagen I in each WT and TSP2KO ECM or hydrogel were confirmed via Western blotting. Briefly, equal amounts of protein extracts (the 50 μg/lane) were separated by SDS-PAGE and transferred to the nitrocellulose membrane (Bio-Rad). Nonspecific binding was blocked with TBST buffer (50 mM Tris/HCl and 150 mM NaCl/0.05% Tween-20) containing 5% (w/v) BSA for 2 hours at room temperature. The membranes were then incubated with primary antibodies for TSP2 (1:250, Customized mouse TSP2 antibody, GenScript) and Collagen I (1:1000, AB765P, EMD Millipore) overnight at 4° C. After washing with TBST three times, the membranes were incubated for 1 hour at 37° C. with secondary antibody conjugated with the fluorescent label (a21109, Invitrogen) diluted 1:1000 in TBST. Finally, the membranes were washed with TBST six times and observed by Infrared Imaging System (Licor Odyssey CLx).

Morphology and Mechanical Properties of the Hydrogels. The SEM images were employed to assess the microstructure for the two hydrogel types. Firstly, the hydrogels were fixed with 2.5% glutaraldehyde. The specimens were then dehydrated for roughly 15 minutes respectively to adjust for their concentration gradient (i.e., 30, 50, 70, 90 and 100% ethanol). After drying, the specimens were coated with Iridium at a thickness of 8 nm and were observed by SEM (SU7000, Hitachi). The fibril curvature and the fibril diameter of the two types of hydrogels were calculated by Image J according to the SEM images.

The storage modulus and loss modulus for both hydrogels at 10 mg/mL were tested by an AR2000 rheometer (TA Instruments) with a 25 mm parallel plate geometry. Gap height was set to 700 μm. The ECM pre-gel was pipetted onto the rheometer plate, which was maintained at a temperature of 10° C. using a Peltier temperature controller. Mineral oil was added to the edge to reduce the evaporation of the samples. The temperature was increased stepwise by 1° C. and allowed to stabilize for 15 s before a measurement was taken. This procedure was followed until the temperature reached 37° C., at which point the temperature was maintained to induce gelation. A frequency of 1 Hz and 1% strain were used to conduct measurements.

Cell Interactions with Hydrogels. Cells proliferation on the surface of the hydrogel was examined to test the cytocompatibility of the WT and TSP2KO hydrogel. A total of 2×10³ C2C12 muscle cells, NIH3T3 fibroblasts or HUVECs were seeded and then cultured for 5 days on either WT or TSP2KO derived hydrogels, respectively. The proliferation was analyzed every day for 5 days using Cell Counting Kit-8 (CCK-8, ab228554, Abcam). Accompanied to the CCK-8 test, the cells were fixed by 4% paraformaldehyde (PFA, J. T. Baker), and then stained with DAPI (Invitrogen) and rhodamine-phalloidin (Invitrogen).

A total of 1×10⁵ C2C12 cells were seeded and cultured on a 10 mg/mL WT or TSP2KO hydrogel for 2 and 4 hours in a 48-well culture plate. A total of 1×10⁵ NIH3T3 cells were seeded and cultured on a 2 mg/mL WT or TSP2KO hydrogel for 2 hours. The hydrogels were rinsed with PBS to remove the unattached cells, and then the specimens were fixed, dehydrated. After drying and coated with Iridium, SEM was used to exam the specimens.

To probe mechanisms of enhanced cell presence into TSP2KO hydrogel, an in vitro assay was used to compare cell invasion through WT and TSP2KO hydrogel. Briefly, 10 mg/mL pre-gel was added into the top chamber of a Transwell (Corning) and placed in an incubator at 37° C. for Ih. After the gel formed, 5×10⁴ C2C12 cells in 100 AL serum-free media were seeded into the top chamber of a Transwell. The cell culture media with 10% FBS was added below the Transwells, and cells could migrate at 37° C. for 6, 8, 12, 48 and 72 h, respectively. A total of 5×10⁴ NIH3T3 cells were seeded and cultured on the 2 mg/ml hydrogels at 37° C. for 4 and 12 h according to the above method. After that, the samples were washed to remove the unattached cells and then were fixed with z-fix overnight. A 70% ethanol solution was used to exchange the z-fix and the samples were embedded in paraffin for sectioning. The sections were stained with H&E according to standard protocols. The C2C12 and NIH3T3 cells invasion distance was calculated by Image J and it was determined that each group had 72 cells at 24 cells per sample.

In vitro tube formation on both hydrogels was observed to evaluate the potential ability of vessels formation in vivo. A total of 1×10⁴ HUVECs were seeded and cultured on WT or TSP2KO hydrogel for 24 hours. The samples were then washed to remove the unattached cells, fixed with 4% PFA overnight, washed with PBS three times and then incubated with primary antibodies for Fibronectin (1:100, ab23750, Abcam) overnight at 4° C. After washing with PBS three times, the samples were incubated for 1 hour at 37° C. with a secondary antibody (Alexa Fluor 488, Invitrogen). Finally, the samples were stained with DAPI (blue) and rhodamine-phalloidin (red). The number and the diameter of the formed tubes were calculated by Image J according to the fluorescent images.

Murine immortalized bone marrow macrophages (iBMM) were cultured as previous protocol in IMDM media (Gibco) containing 20% FBS (Gibco), 2.5 mM L-glutamine (Gibco), 1% Penicillin-Streptomycin (CAISSON), and 50 ng/ml M-CSF (R&D Systems). For the macrophage polarization assay, the bottom of the 6-well culture plate was coated with hydrogel firstly. Macrophages at a density of 2×10⁵/well were cultured on the WT and TSP2KO hydrogel for 3, 7 and 14 days, respectively. Fluorescence microscopy was employed to visualize the morphology of the macrophages. Quantitative PCR was used to analyze the expression of CD8β, IL-1β, Tnfα, Tgfβ, IL-10, and Arg1. Gapdh was used as an internal control. RT-qPCR primers are shown in Table 1.

TABLE 1
RT-qPCR primers

| Gene | Primers |
|---|---|
| CD86 | 5′-TCAATGGGACTGCATATCTGCC-3′ |
| | (F) (SEQ ID NO: 1) |
| | 5′-GCCAAAATACTACCAGCTCACT-3′ |
| | (R) (SEQ ID NO: 2) |
| IL-1 β | 5′-CAACCAACAAGTGATATTCTCC-3′ |
| | (F) (SEQ ID NO: 3) |
| | 5′-GATCCACACTCTCCAGCTGCA-3′ |
| | (R) (SEQ ID NO: 4) |
| Tnf α | 5′-CATCTTCTCAAAATTCGAGTGACAA-3′ |
| | (F) (SEQ ID NO: 5) |
| | 5′-TGGGAGTAGACAAGGTACAAC-3′ |
| | (R) (SEQ ID NO: 6) |
| Tgf β | 5′-TGACGTCACTGGAGTTGTACGG-3′ |
| | (F) (SEQ ID NO: 7) |
| | 5′-GGTTCATGTCATGGATGGTGC-3′ |
| | (R) (SEQ ID NO: 8) |
| IL-10 | 5′-GCTCTTACTGACTGGCATGAG-3′ |
| | (F) (SEQ ID NO: 9) |
| | 5′-CGCAGCTCTAGGAGCATGTG-3′ |
| | (R) (SEQ ID NO: 10) |
| Arg1 | 5′-CTCCAAGCCAAAGTCCTTAGAG-3′ |
| | (F) (SEQ ID NO: 11) |
| | 5′-AGGAGCTGTCATTAGGGACATC-3′ |
| | (R) (SEQ ID NO: 12) |
| Gapdh | 5′-TTCACCACCATGGAGAAGGC-3′ |
| | (F) (SEQ ID NO: 13) |
| | 5′-GGCATGGACTGTGGTCATGA-3′ |
| | (R) (SEQ ID NO: 14) |

Subcutaneous Injection, Subcutaneous injection of tissue-derived hydrogels was performed by injecting 250 μL of pre-gel solution (kept on ice) subcutaneously for 5 days or 10 days in 12-week-old C57BL/6J mice. Each mouse received two injections in its dorsal region, each from a different genotype of gel (WT, KO). Implants were excised with the surrounding tissue intact, fixed in Z-fix, and embedded in paraffin for sectioning. Sections were stained with H&E, as well as for CD31 (1:50, AF3628, R&D Systems), α-SMA (1:200, ab5694, Abcam), Neurofilament H (1:200, AB1989, EMD Millipore), Vimentin (1:500. AB5733, EMD Millipore), F4/80 (1:200, NB600-404SS, Novus) and CD 3e (1:200, 553058, BD Pharma) immunohistochemically (Vector, ABC kit and Peroxidase substrate kit). For analysis of cell penetration, eight 20× images were taken per injection (n=5), and the average number of cells per high-power field was quantified.

Volumetric Muscle Loss Surgery. Twelve-week-old wild-type C57BL/6J mice were anesthetized with 4% isoflurane and maintained under 2.5% isoflurane. Hair was removed from the lower extremity. After ethanol sterilization of the surrounding skin, a 1.5-cm incision was created between the knee and hip joint to access the quadriceps femoris muscle. A 2×7×2 mm piece of the muscle was removed by a surgical scissor. After creating the VML, the hydrogel (n=5/group, 200 μl each site) was implanted directly into the injury site, and then muscle and skin incisions were sutured layer by layer using 8-0 suture (J975G, VICRYL™, ETHICON®) and 6-0 suture (8695G, PROLENE™, ETHICON®), respectively. Buprenorphine (0.05-0.1 mg/kg) and Rimadyl (5.0 mg/kg) were administered.

After surgery for 3, 7, 14 and 28 days, animals were euthanized, and the specimens were excised and prepared for H&E and Trichrome staining. Additionally, samples were analyzed via immunohistochemistry with anti-Laminin (1:200, ab11575, Abcam), anti-Vimentin (1:500, AB5733, EMD Millipore), anti-F4/80 (1:200, NB600-404SS, Novus), anti-CD3e (1:200, 553058, BD Pharma), anti-CD31 (1:50, AF3628, R&D Systems), anti-αSMA (1:50, AF3628, R&D Systems) and anti-Neurofilament H (1:200, AB1989, EMD Millipore) antibodies. For quantifying the cellular content of hydrogels after implantation in muscle, ImageJ was used to quantify the number of cells (n=3) and CD31+−αSMA+lumens (n=5). Eight 20×images were quantified per implant. Neurocytes in the newly formed tissues were confirmed with anti-Neurofilament H antibody (1:1000, AB1989, EMD Millipore) via Western blotting. GAPDH was used as an internal control (1:1000, 14C10, Cell Signaling). The integrated density of the protein band was analyzed by Image J (n=3).

Treadmill Testing for Muscle Function. Mice were acclimatized to running on a Columbus Instruments Exer3/6 treadmill twice a week for two weeks. For all functional analyses, tests were performed in all groups at the same time of day as well as same day of the week for standardization purposes. Mice were unrestricted in terms of activity post-surgery. Analyses were performed 4 days pre-op and at 3, 7, 14, and 28 post-op. For the maximal speed test, mice ran at 5 m/min for 5 min, after which the speed was increased by 1 m/min every minute. A low voltage was applied to a grate at the end of the treadmill to encourage the mice to continue running for the duration. Mice were run to exhaustion, defined as when the mouse stayed on the pulsed shock grid for a continuous 30 seconds and was unresponsive to three hand brushes. The max speed was recorded as the max speed attained prior to exhaustion.

The assessment of voluntary ambulation in a mouse model is a non-invasive and reproducible activity assay such as the 6-minute walk test and related mobility scores. A six-minute video of a mouse voluntarily moving around its cage, which was performed in a quiet, temperature-controlled room. Adobe Premier Rush is the program used to ensure each video was exactly 6 minutes in length. Then a video analysis software (Kinovea) was used to track the mice in the ambulation test field and quantify the distance traveled.

Statistical Analysis. Data are expressed as mean #standard error of the mean (SEM). All statistical analyses of data with more than two samples were conducted using one-way ANOVA with Tukey's multiple comparison test. For analysis of cell penetration into WT, and TSP2KO hydrogels at various depths, a two-way ANOVA with Tukey's multiple comparison test was used. Values of p<0.05 were considered statistically significant.

The results of the experiments are now described.

### Preparation and Characterization of Muscle ECM Hydrogels.

WT and TSP2KO muscle ECM hydrogels were prepared via a step-wise process outlined in FIG. 1A with a focus on preventing ECM damage and preserving proteins. To confirm efficient decellularization and maintenance of ECM architecture processed tissues were stained with H&E and Masson's trichrome (FIG. 1B), analyzed for DNA content (FIG. 1C), and protein content (FIGS. 1D-1F). Histological analysis revealed the lack of cells and this was supported by the low DNA content in decellularized ECM and hydrogels. Specifically, WT and TSP2KO hydrogels contained (2.14±0.99 ng/mg) and (2.54±1.41 ng/mg), respectively, which is far below the suggested threshold of 50 ng DNA/mg ECM for decellularized constructs. Moreover, SDS/PAGE revealed the retention of proteins during the decellularization process that was similar between WT and TSP2KO samples (FIG. 1D). Western blot analyses of hydrogel protein extracts with anti-TSP2 or anti-Collagen antibodies confirmed the absence of TSP2 in TSP2KO hydrogels (FIG. 1E) and the presence of the α (115~130 kD), β (~200 kD), γ (>250 kD) chains of collagen in both samples (FIG. 1F). Additionally, proteomic analysis identified 10 extracellular matrix proteins that were differentially expressed between WT and TSP2KO muscle ECM (FIGS. 1K-IM). The majority of these were collagen-related proteins (FIG. 1K). Specifically, Col2a1 and Col11a2 were highly expressed in the KO samples while Col3a1, Col4a4, Col5a1, and Col5a2 were lower (*p<0.05, FIG. 1K, FIG. 1M). However, the major structural proteins like Collagen type I showed similar expression level between the two genotypes (FIGS. 1K-1M).

Prepared hydrogels were also visualized by scanning electron microscopy (SEM) as shown in FIG. 1G. Image analysis revealed that TSP2KO hydrogels contained collagen fibrils that were equal to WT in diameter but displayed greater curvature (FIGS. 1H-1I). Finally, rheology showed that TSP2KO hydrogels had altered biomechanical properties with a lower storage modulus (FIG. 1J).

Cell Interactions with Muscle ECM Hydrogels.

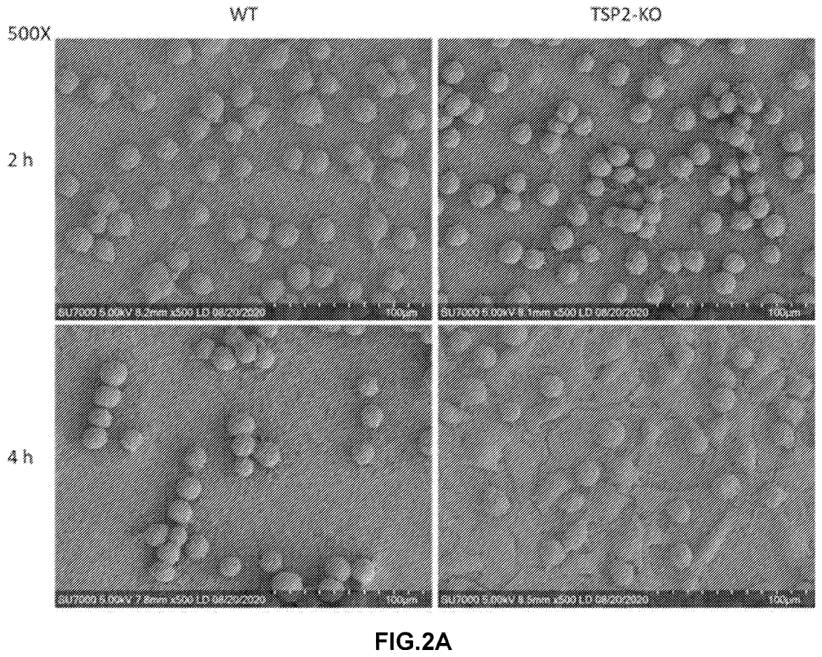

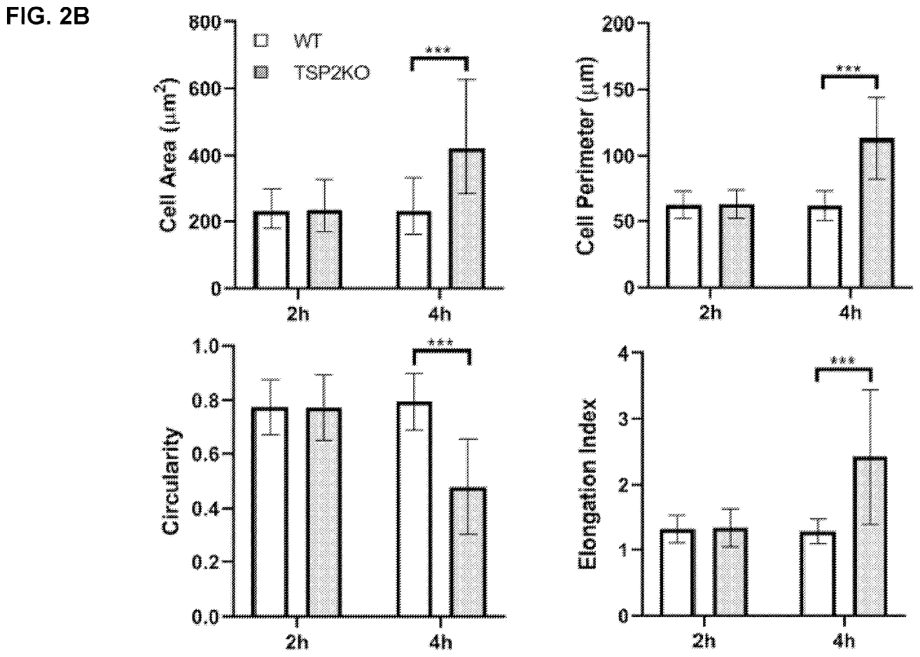

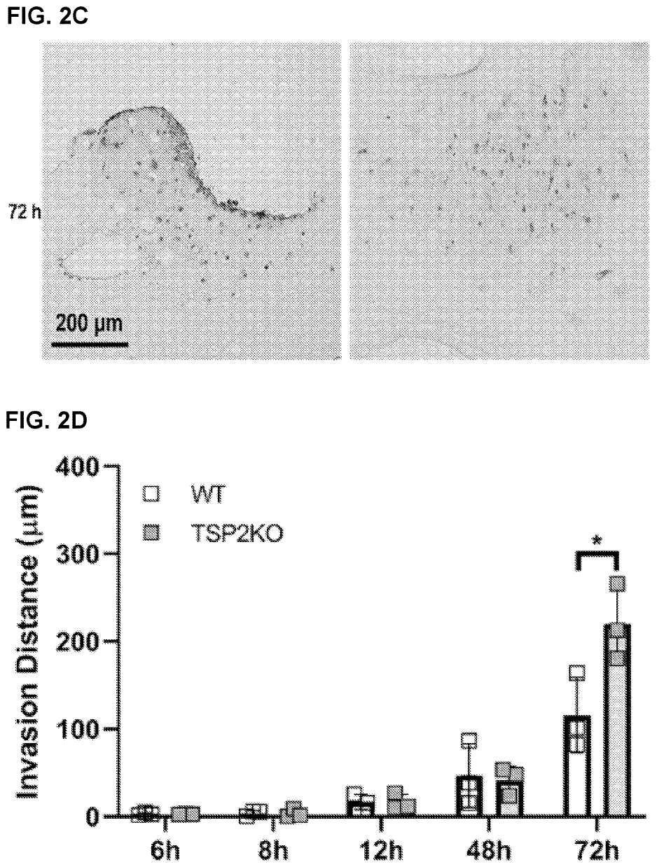

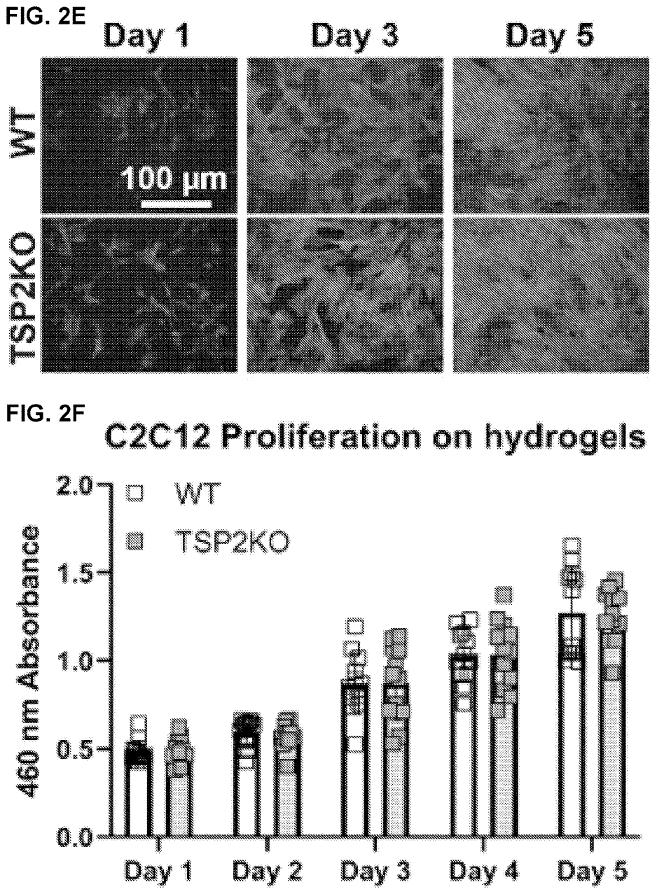

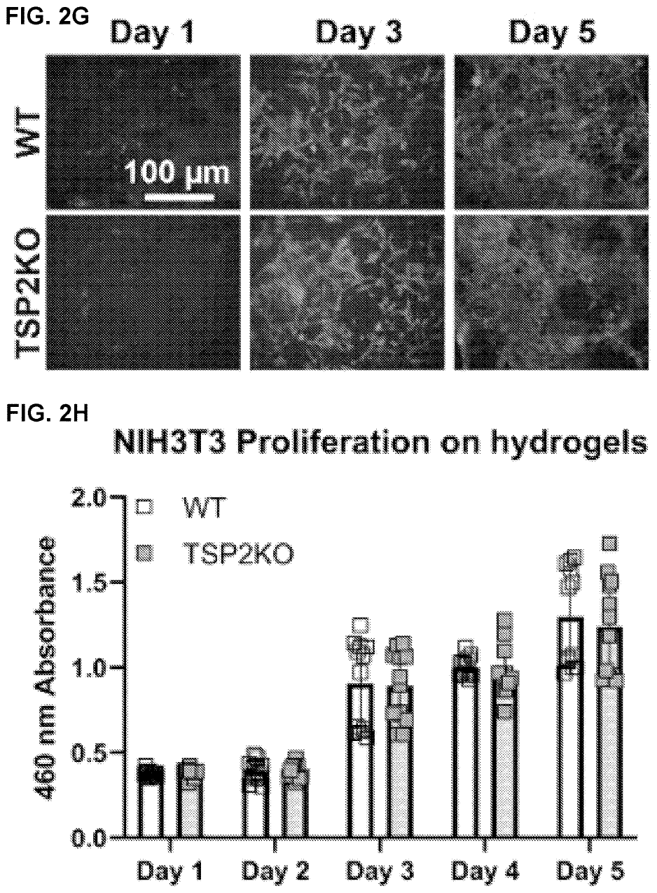

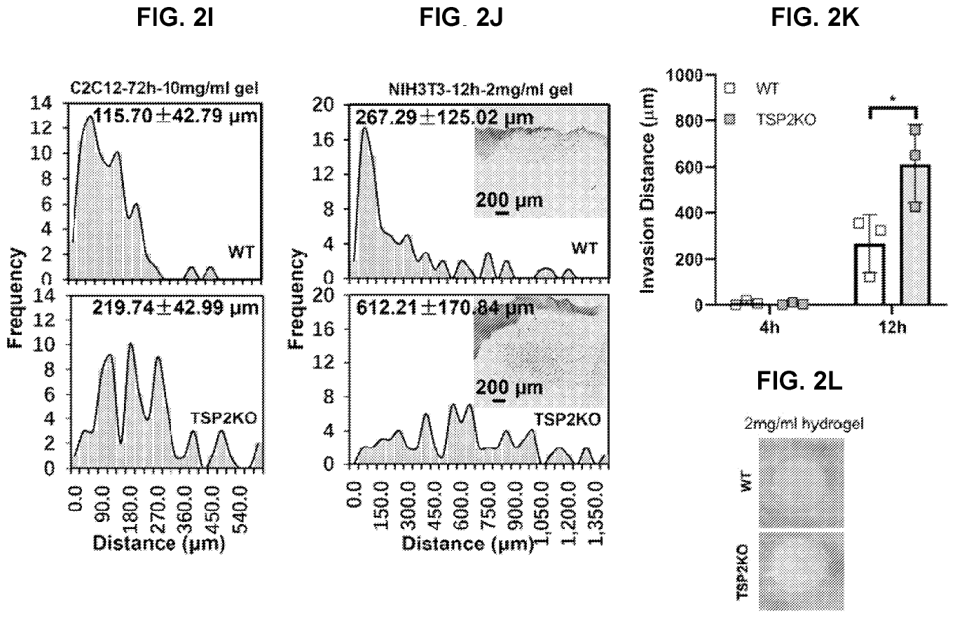

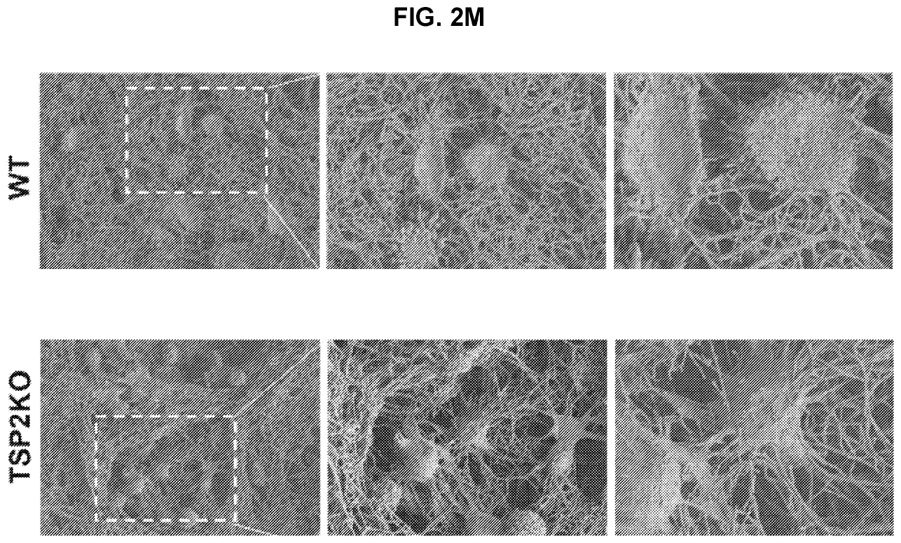

To assess the ability of hydrogels to support cell functions relevant to responses to injury, the morphology, proliferation and migration of C2C12 on WT or TSP2KO hydrogels (10 mg/ml) was analyzed. No differences in proliferation were detected (FIGS. 2E-2F). SEM visualization and image analysis revealed that C2C12 cells spread faster on TSP2KO hydrogels and by 4 hrs displayed larger cell area and perimeter and were less circular and more elongated in shape (FIGS. 2A-2B). Cell migration was investigated in a modified transwell assay where C2C12 were placed on pre-formed hydrogels and their invasion into the hydrogels was measured up to 72 hrs by histological analysis as shown in FIG. 2C. Measurements revealed greater invasion of C2C12 cells into TSP2KO hydrogels after 72 hrs (FIG. 2D).

To investigate the responses of other cell types, we utilized NIH3T3 cells as a suitable fibroblast cell type. Similar to C2C12 cells, observed differences in proliferation were not observed when cultured on WT or TSP2KO hydrogels (FIGS. 2G-2H). To evaluate migration, NIH3T3 were evaluated as described above with the exception that the concentration of hydrogel was lowered to 2 mg/ml due to insufficient invasion at higher hydrogel densities (not shown). Measurements revealed increased migration into TSP2KO hydrogels at 12 hrs post plating (FIGS. 2J-2K). SEM analysis at 2 hrs post plating showed that NIH3T3 cells were more spread and displayed extensive interactions and integration with the ECM (FIG. 2L).

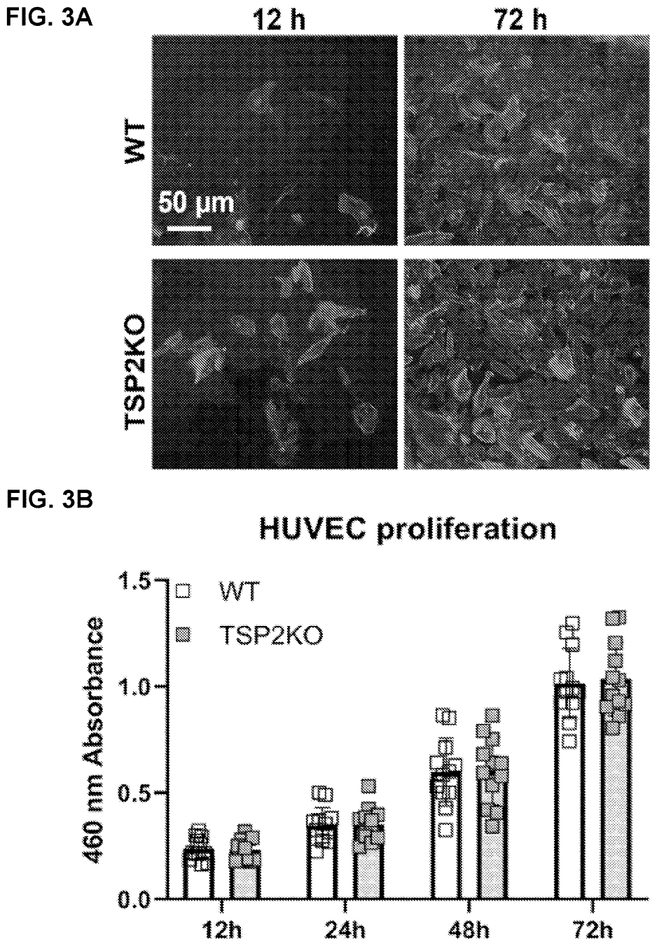

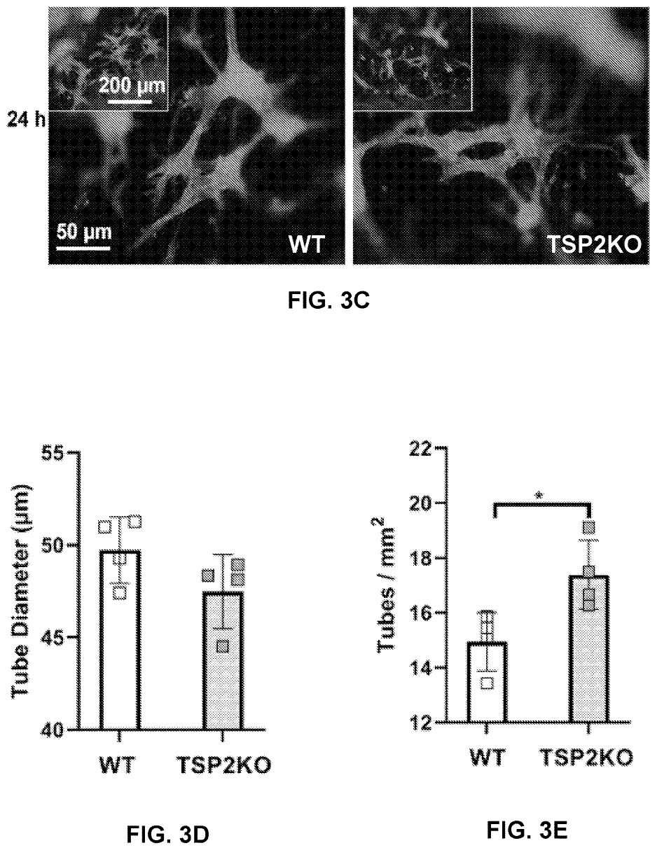

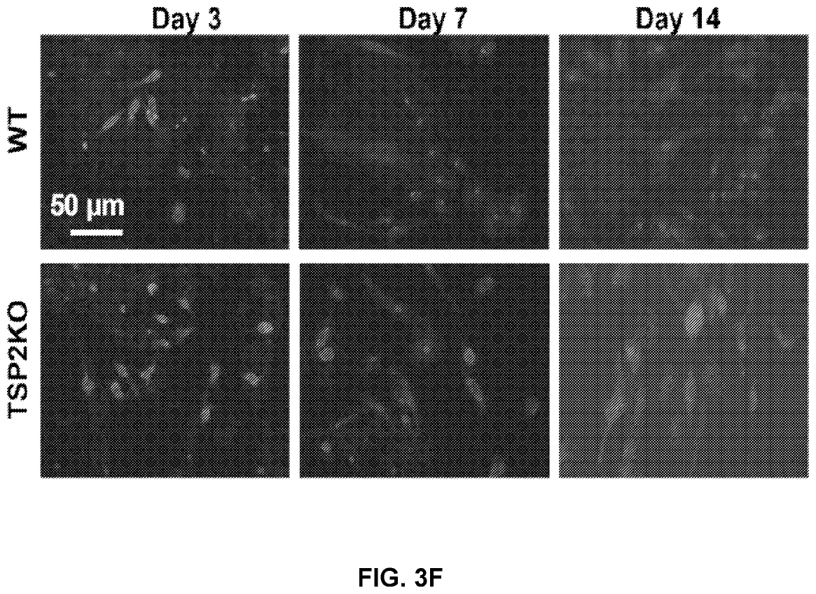

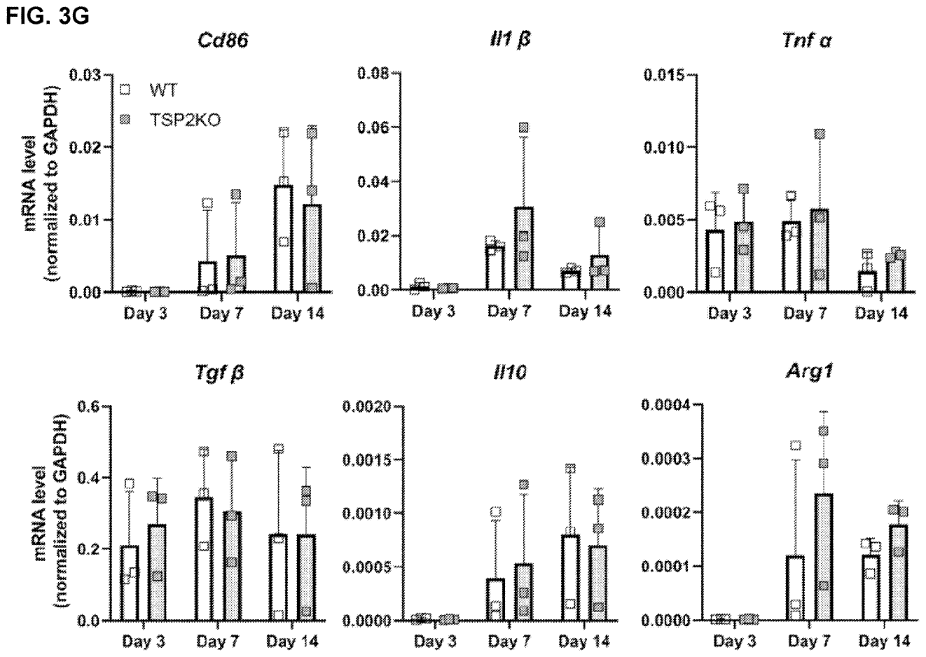

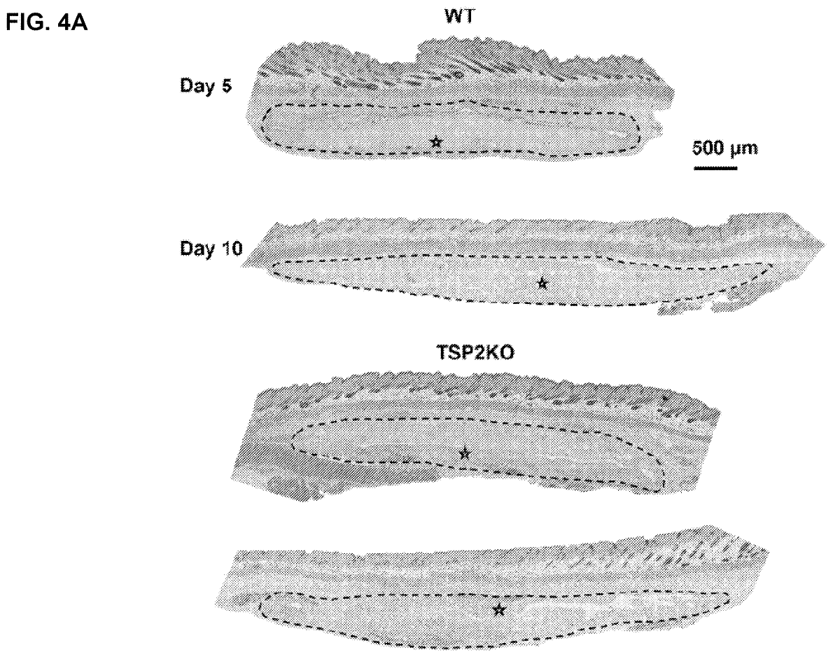

To further explore cell-hydrogel interactions relevant to injury responses, we utilized endothelial cells (HUVEC) and macrophages (murine immortalized bone marrow-derived macrophages; iBMM) to probe possible angiogenic and immune responses, respectively. Proliferation and morphology of HUVEC was monitored for 72 hrs and was found to be similar on both hydrogels (FIGS. 3A-3B). However, when mixed within WT or TSP2KO hydrogels in a tube formation assay, HUVEC formed a greater number of tubes of similar diameter in the TSP2KO hydrogel (FIGS. 3C-3E). iBMM cultured on either WT or TSP2KO hydrogels for up to 14 days exhibited similar densities and morphology (FIG. 3F). Possible activation and polarization of macrophages during the culture period was monitored by analyzing the expression of key cytokines and growth factors (CD8β, IL-1β, TNF, TGF-β, IL-10, and Arg-1) by qRT-PCR and was found to be similar (FIG. 3G). These in vitro observations suggested that when compared to WT, TSP2KO hydrogels could increase angiogenesis without altering inflammatory responses.

### Biocompatibility of Muscle ECM Hydrogels.

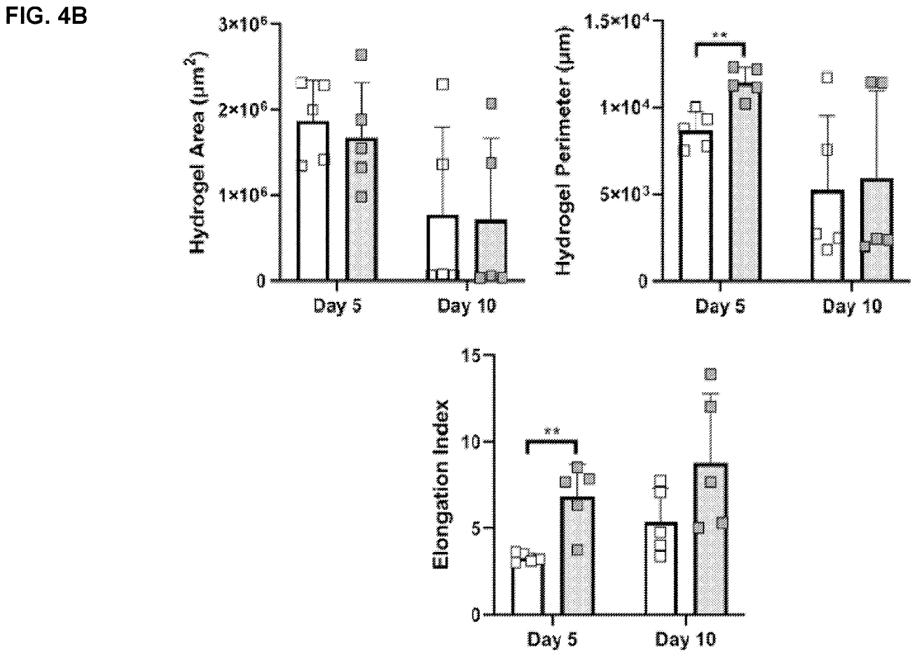

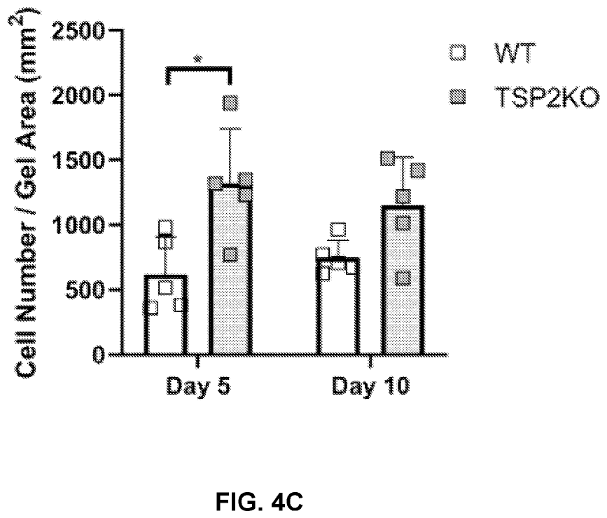

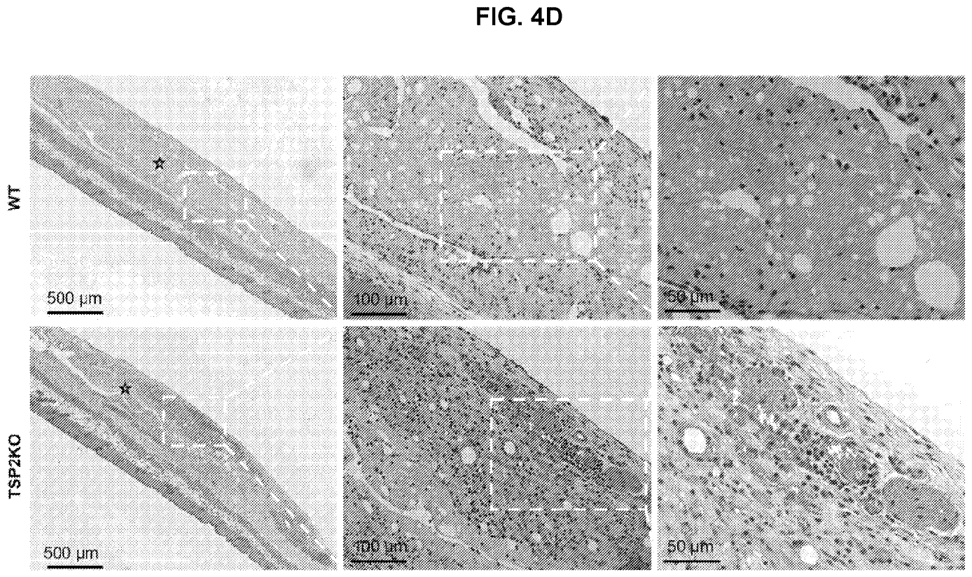

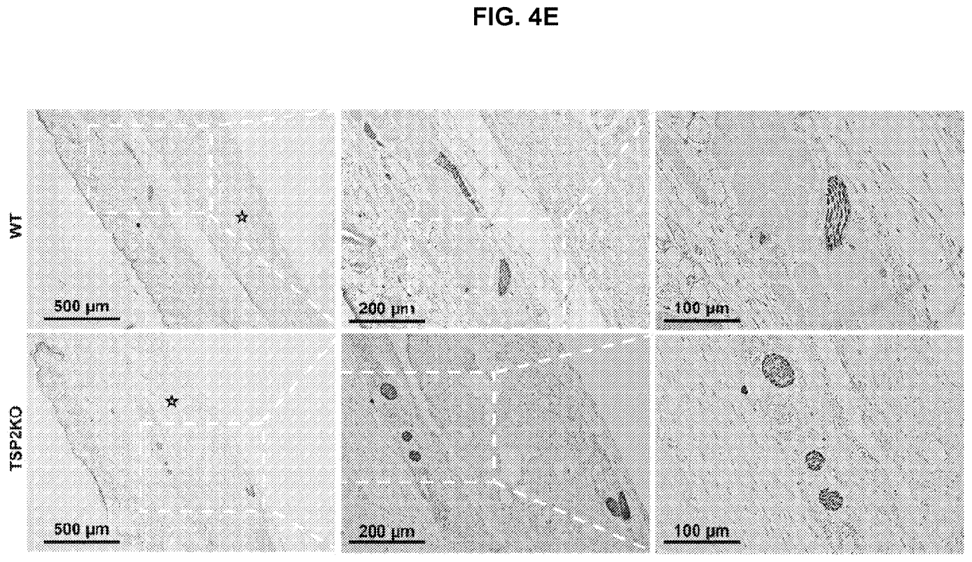

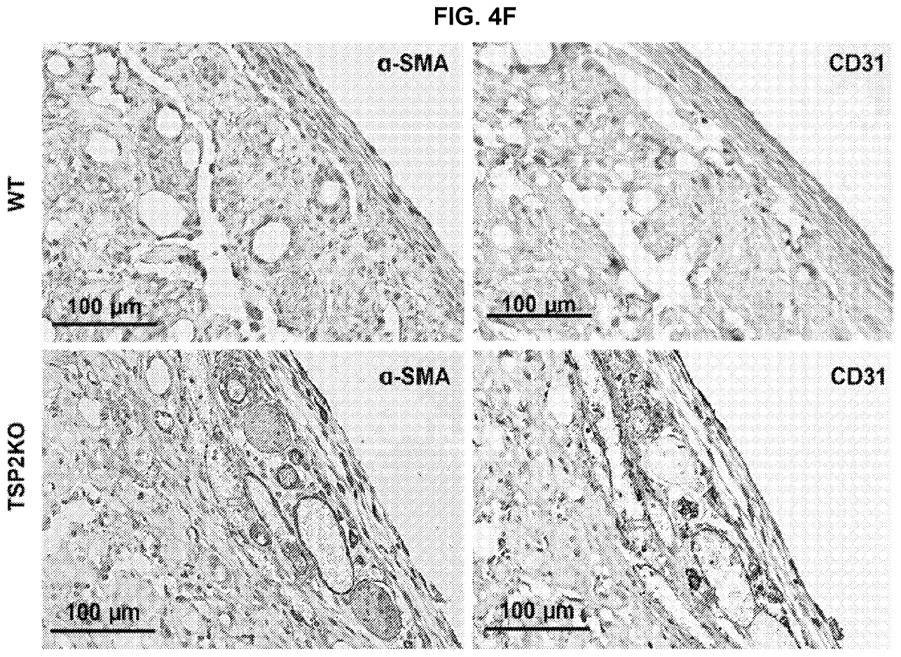

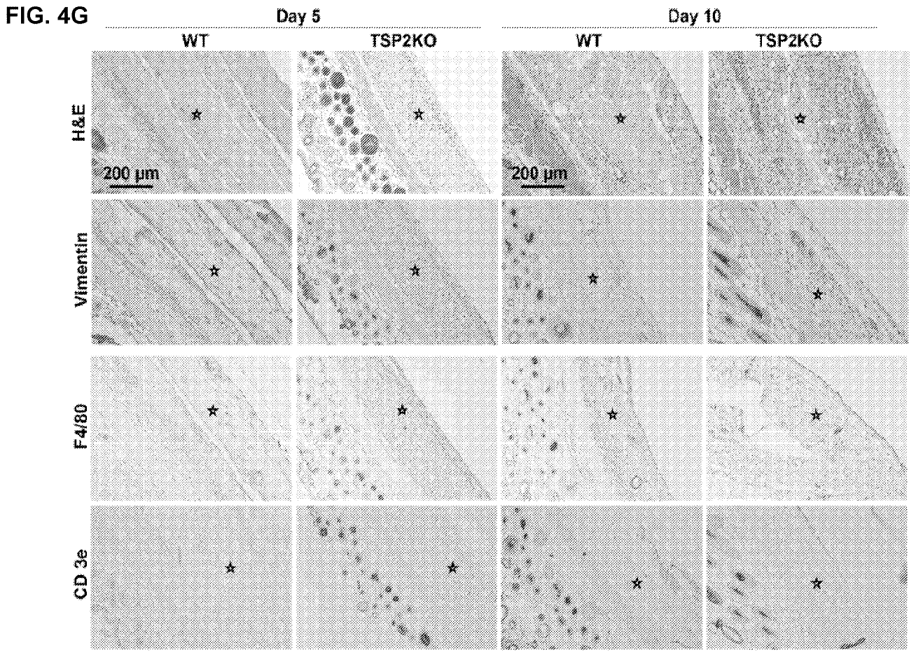

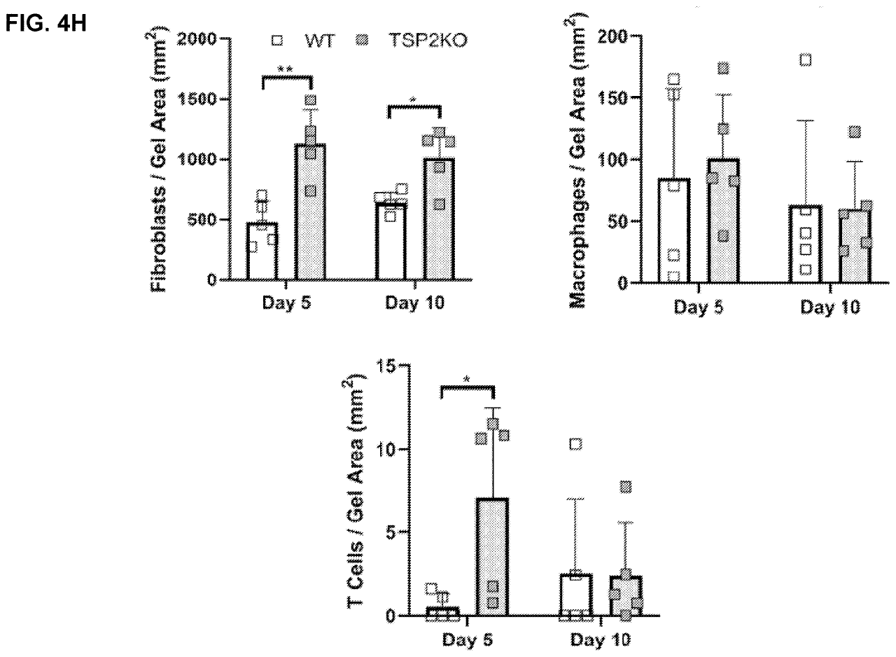

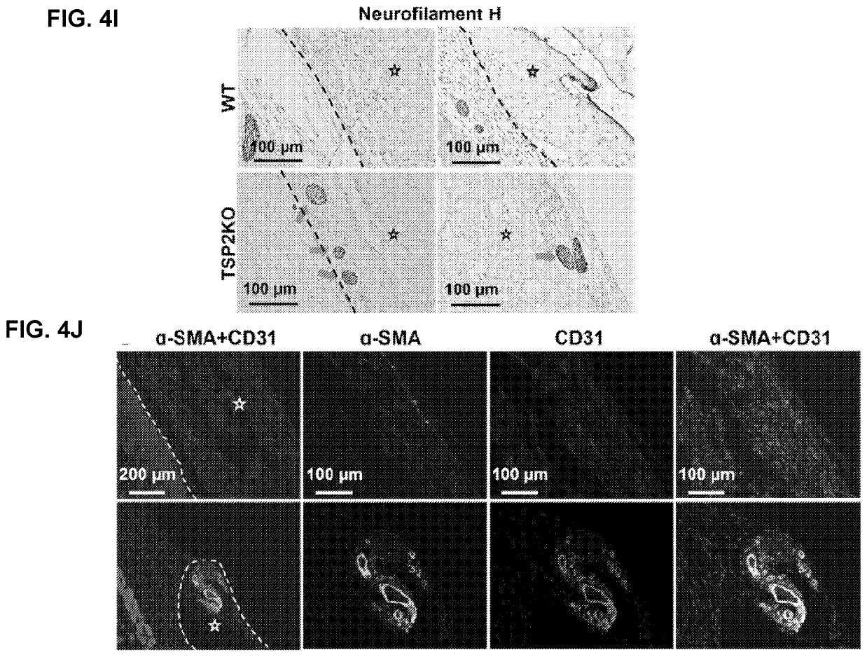

Subcutaneous (SC) injections were performed to assess the overall biocompatibility of WT and TSP2KO hydrogels. Specifically, ECM solutions were prepared and injected SC into mice to form hydrogels in situ, which were retrieved 5 or 10 days later. Histological evaluation following H&E stain of hydrogel sections revealed the progressive decrease in hydrogel area with the TSP2KO hydrogels displaying more elongated shape (FIGS. 4A-4B). In addition, more cells were observed within TSP2KO hydrogels at day 5 (FIG. 4C). The samples subcutaneously implanted in the mice were visualized by H&E staining 10 days post implantation (FIG. 4D). Immunochemical staining (Neurofilament H) indicated that the TSP2KO hydrogel had nerves present 10 days after subcutaneous injection in mice but no nerves were present in the WT hydrogel (FIG. 4E). Immunochemical staining (α-SMA and CD31) indicated that the TSP2KO hydrogel had blood vessels present 10 days after subcutaneous injection in mice (FIG. 4F). Immunohistochemistry was employed to identify vimentin-positive cells as well as macrophages (F4/80 Ab) and T-cells (pan T-cell CD3e Ab) (FIG. 4G). Histomorphometric analyses revealed that TSP2KO hydrogels contained higher number of vimentin-positive cells (day 5 and 10) and T-cells (day 5) (FIG. 4H). No differences in the number of macrophages were observed, which is consistent with the in vitro observations.

Histological examination suggested an angiogenic response within TSP2KO but not WT hydrogels and immunohistochemistry was employed to detect newly formed blood vessels. Specifically, d 10 sections were stained with antibodies to CD31 and α-SMA to detect endothelial cells and smooth muscle cells, respectively. Consistent with the histological assessment, newly formed CD31 and α-SMA-positive vessels were observed only in TSP2KO hydrogels (FIG. 4J). To evaluate whether neovascularization of hydrogels was coupled with nerve formation, sections were also stained with the Neurofilament H Ab. Consistent with the observation of an angiogenic response, newly formed nerves were only detected in TSP2KO hydrogels (FIG. 4I). Taken together, these observations suggested that both WT and TSP2KO hydrogels were well tolerated and did not induce excessive inflammation. However, the TSP2KO hydrogel exhibited greater cell content with newly formed vessels and nerves indicative of significantly improved regenerative capacity compared to the WT hydrogel.

### Application of Hydrogel in VML Model.

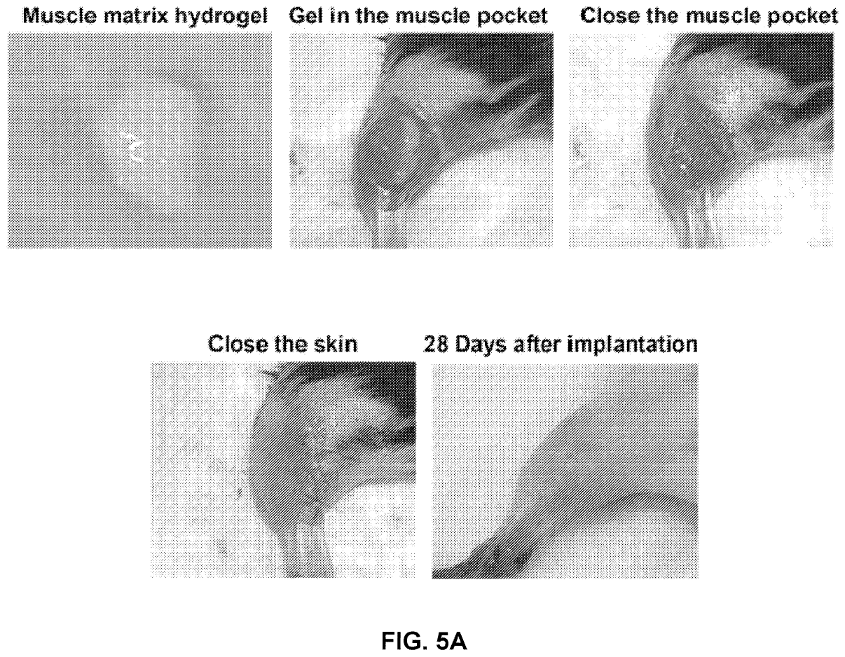

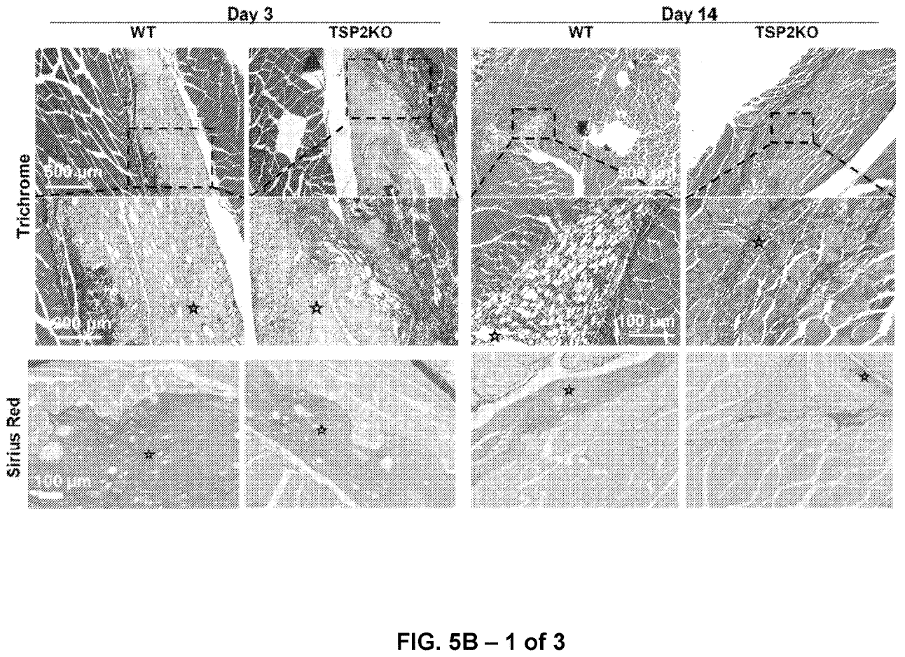

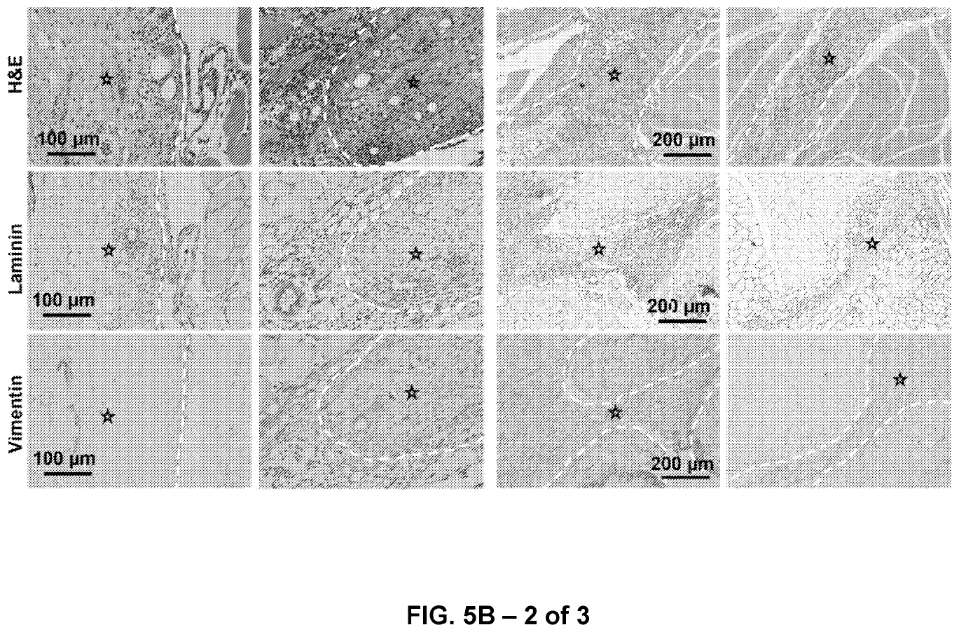

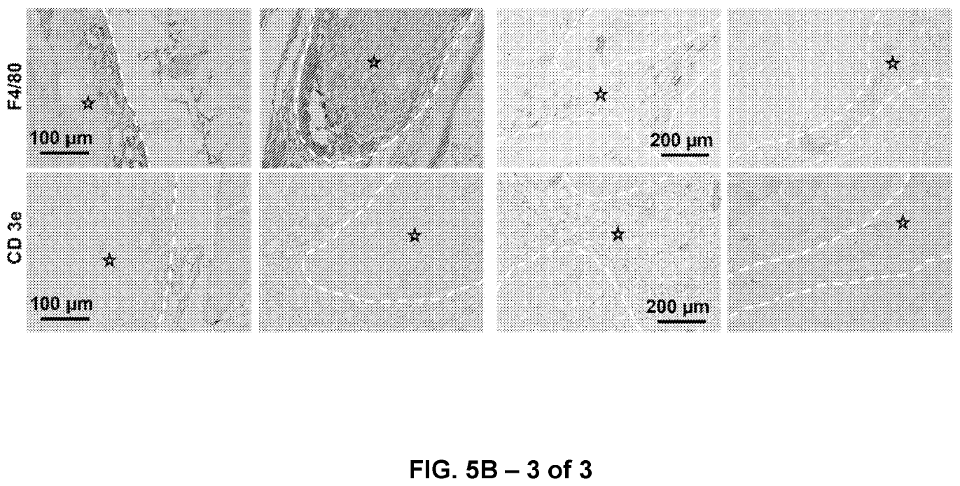

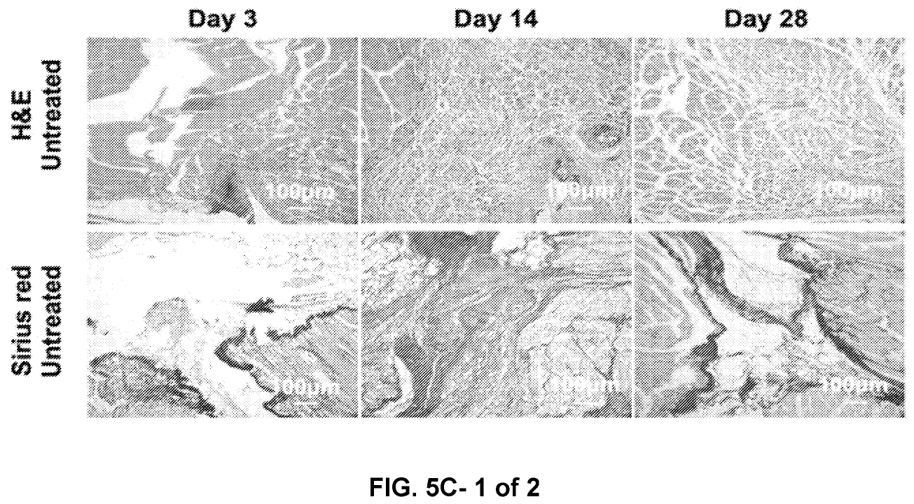

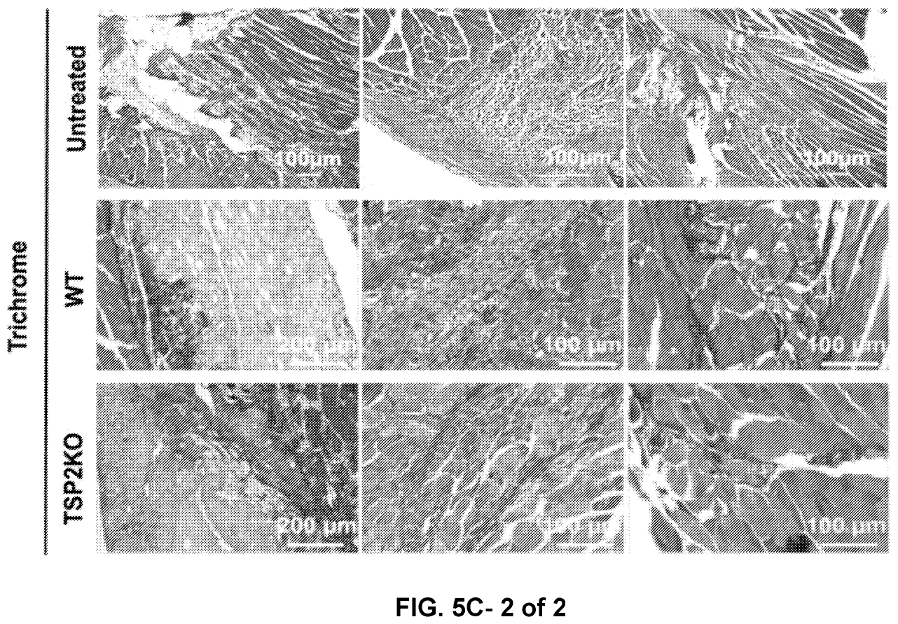

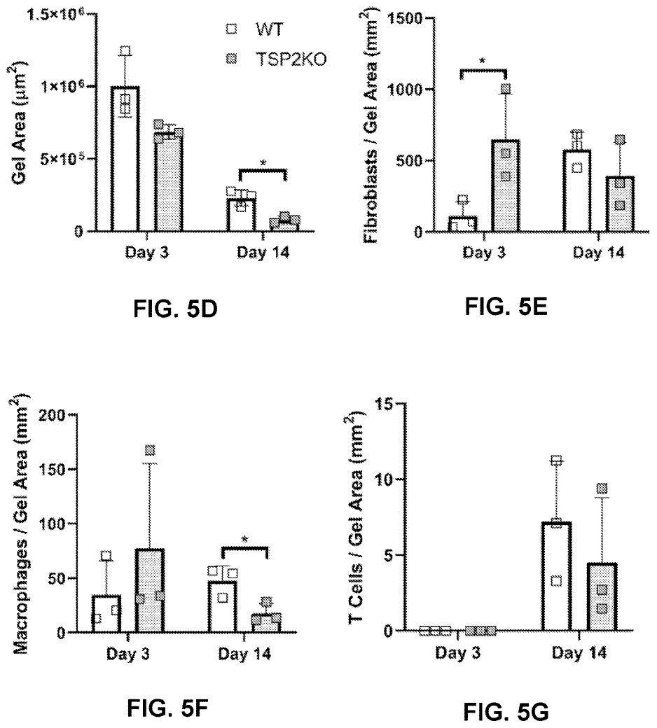

A VML model was created to evaluate muscle hydrogel function following surgical injury. Specifically, WT or TSP2KO preformed hydrogels were placed in a surgical defect and then the muscle and skin were sutured as shown in FIG. 5A. Tissue samples were obtained on day 3, 14 or 28 post injury and analyzed via Trichrome staining (FIGS. 5B-5C). Hydrogel area (blue stain) was measured using Image J and was found to be decreased at d 14 in the case of the TSP2KO (FIG. 5D). Samples were also stained with H&E, which suggested that TSP2KO hydrogels contained more cells at d 3. In addition, immunohistochemical detection of laminin revealed remodeling of the hydrogels with increased deposition at d 14. Immunohistochemical analysis revealed the enhanced presence of vimentin-positive cells (mesenchymal lineage, fibroblasts) in TSP2KO hydrogels (FIG. 5B, FIG. 5E). TSP2KO hydrogels also contained less macrophages at d 14, while no difference in the number of CD3e-positive (Pan T cells) was observed (FIG. 5B and FIGS. 5F-5G).

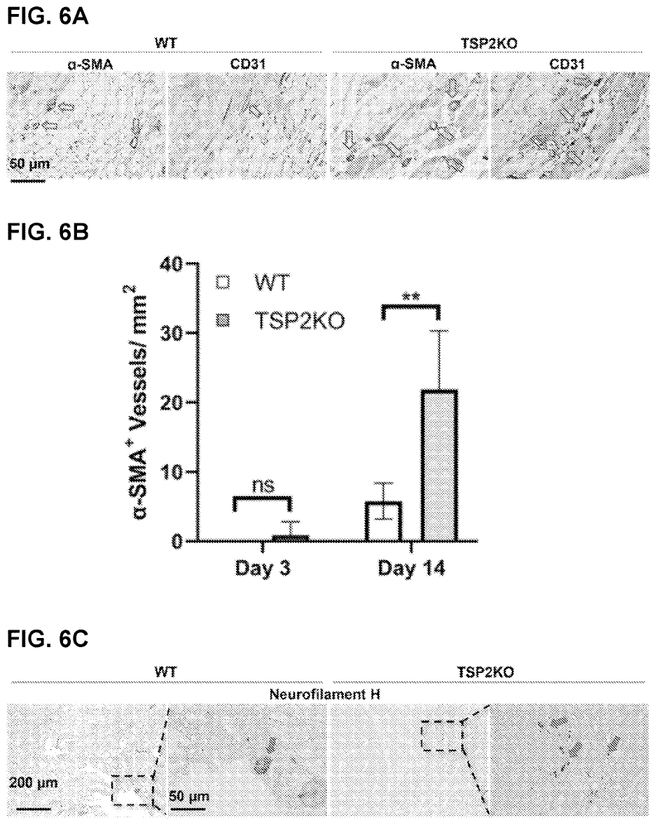

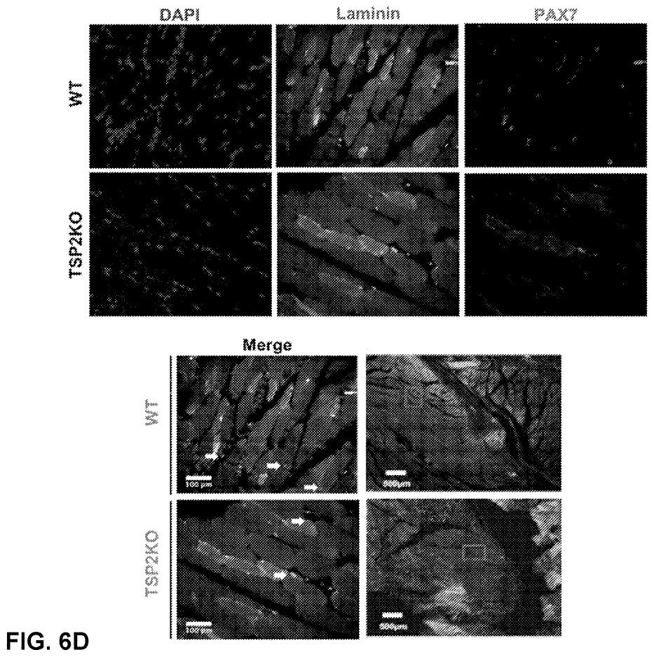

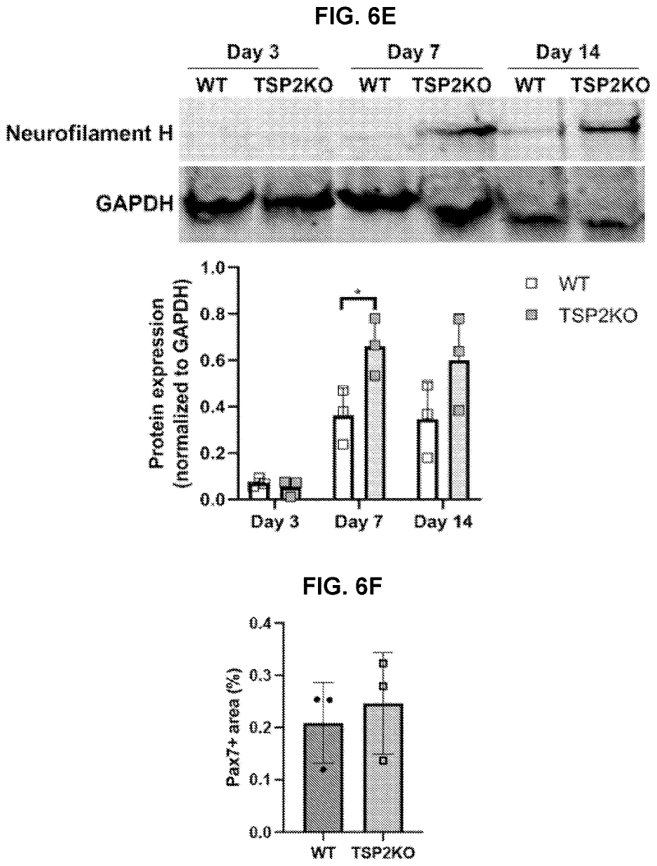

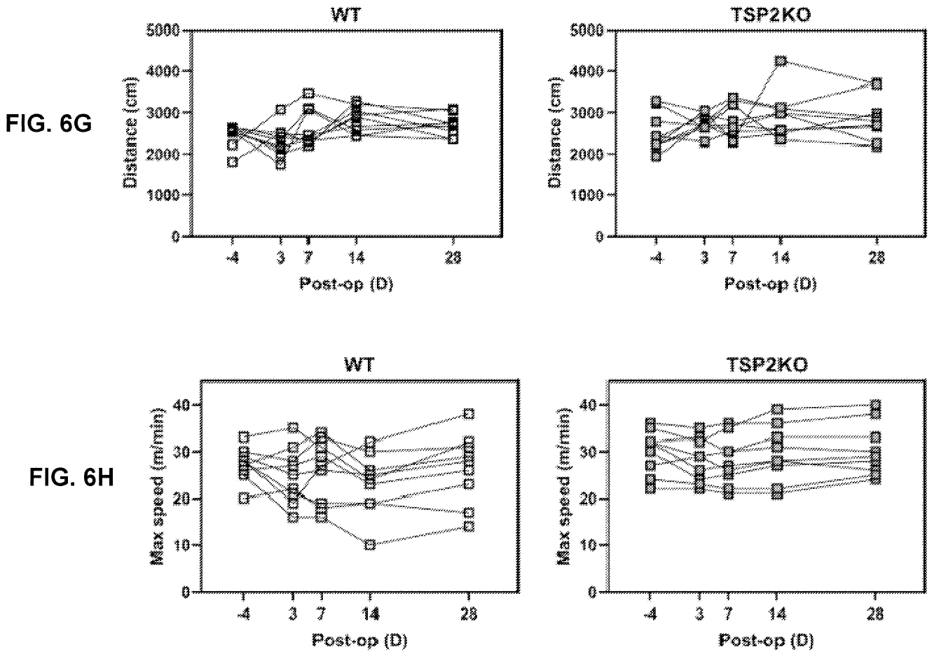

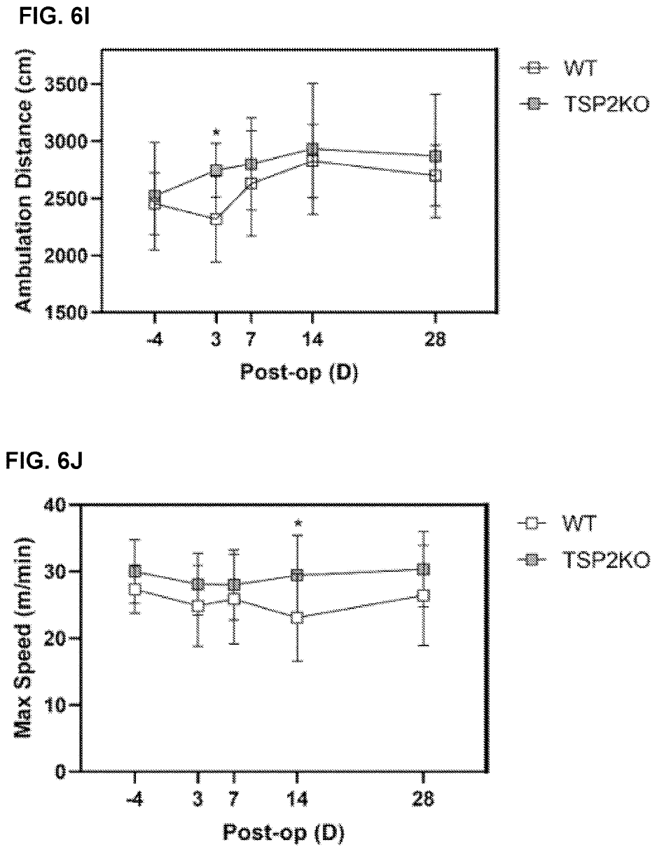

To further evaluate hydrogel-induced remodeling, samples were stained with CD31 and α-SMA Abs to analyze newly formed blood vessels and neurofilament H Ab to detect innervation. Consistent with enhanced presence of repair cells, an increase in mature (α-SMA-positive) blood vessels and innervation in TSP2KO hydrogels were detected at d 14 (FIGS. 6A-6C). Additionally, detection of Laminin and satellite cells (PAX7) by immunofluorescence suggested ongoing remodeling at 14 d (FIG. 6D). Increase in neurofilament H was confirmed by western blot analysis of muscle hydrogel extracts (FIG. 6E).

To evaluate the ability of hydrogels to induce restoration of muscle function, a max speed test and six minute ambulation test were performed. Ambulation distance and max speed were measured at 4 days pre- and 3, 7, 14, and 28 days post-surgery (FIGS. 6F-6G). Immediately post-op, TSP2KO mice exhibited increased voluntary ambulation (*p<0.05, n=9, FIG. 6H). Mice treated with TSP2KO hydrogel implants ran faster (29.4±6.0 m/min, *p<0.05, n=9, FIG. 6I) than mice with WT hydrogel implants (23.1±6.6 m/min) 14 days post-surgery, indicating improved functional recovery. Moreover, monitoring of voluntary ambulation revealed that mice treated with TSP2KO hydrogel covered a greater distance at day 3 post-op. These observations suggest that mice treated with TSP2KO hydrogel induce more robust muscle regeneration allowing them to achieve greater ambulation distance and maximum speed.

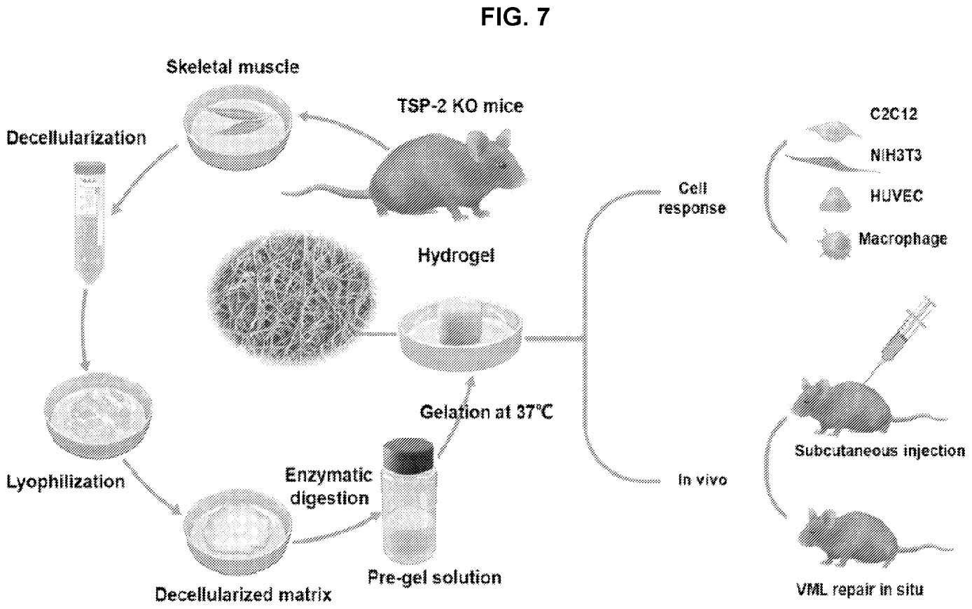

The preparation process for muscle-derived ECM hydrogel and in vivo and in vitro applications thereof is summarized in FIG. 7. Compared to WT ECM hydrogel, mouse muscle-derived TSP2KO ECM hydrogel demonstrated a more curved fibril structure and unique biomechanical properties. The TSP2KO hydrogel, when compared to WT hydrogel, allowed enhanced invasion of C2C12 myocytes and chord formation by endothelial cells and was better able to support C2C12 and NIH3T3 cell adhesion and invasion, HUVEC in vitro tube formation, and in vivo microvascular and nerve formation. Furthermore, it was evident that mice treated with TSP2KO ECM hydrogels were better protected from muscle dysfunction following VML surgery.

As demonstrated herein, muscle-derived TSP2KO ECM hydrogels can induce greater regeneration in the VML model compared to WT ECM hydrogel. VML overwhelms regenerative processes leading to limited repair; thus, utilization of the VML model allowed rigorous evaluation of the hydrogel's therapeutic potential. The TSP2KO ECM hydrogel was demonstrated herein to be biocompatible, biodegradable, and capable of encouraging cell growth, enhanced cell invasion and incorporation with surrounding tissue including formation of new vessels and peripheral nerves in the VML model. Compared to WT ECM hydrogel, the TSP2KO ECM hydrogel induced greater recruitment of repair cells, innervation, and blood vessel formation and reduced inflammation. Moreover, mice treated with TSP2KO hydrogel exhibited a blunted response to injury and ran at greater maximum speed. Taken together, these observations indicate that TSP2KO hydrogels induce muscle regeneration in a VML model.

### Example 2: Preparation and Characterization of ECM Hydrogels Originating from Bone

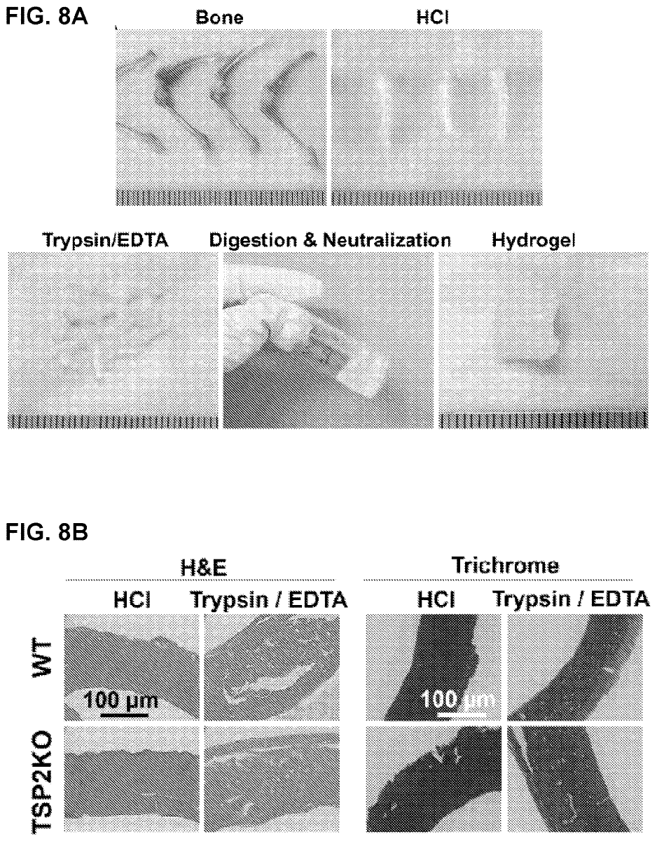

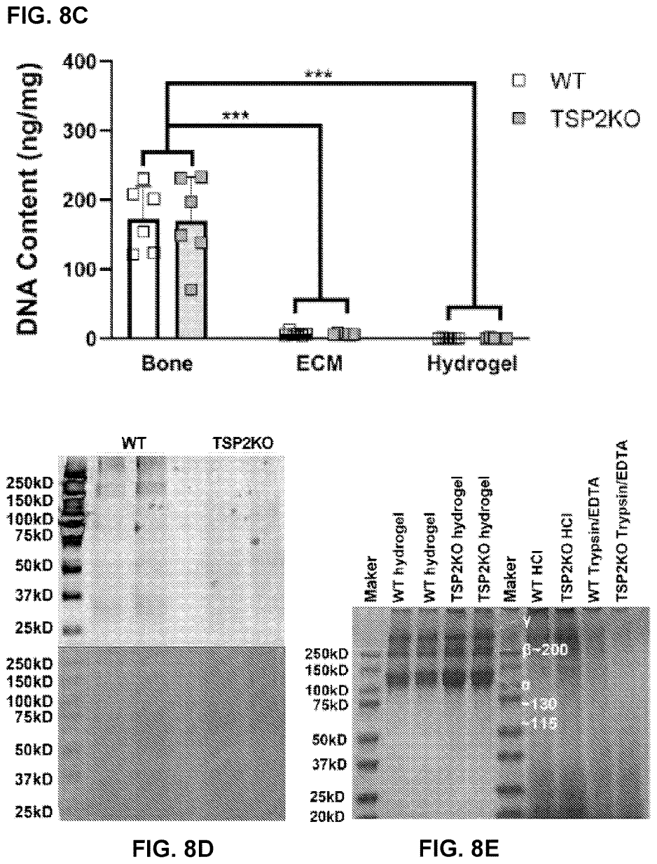

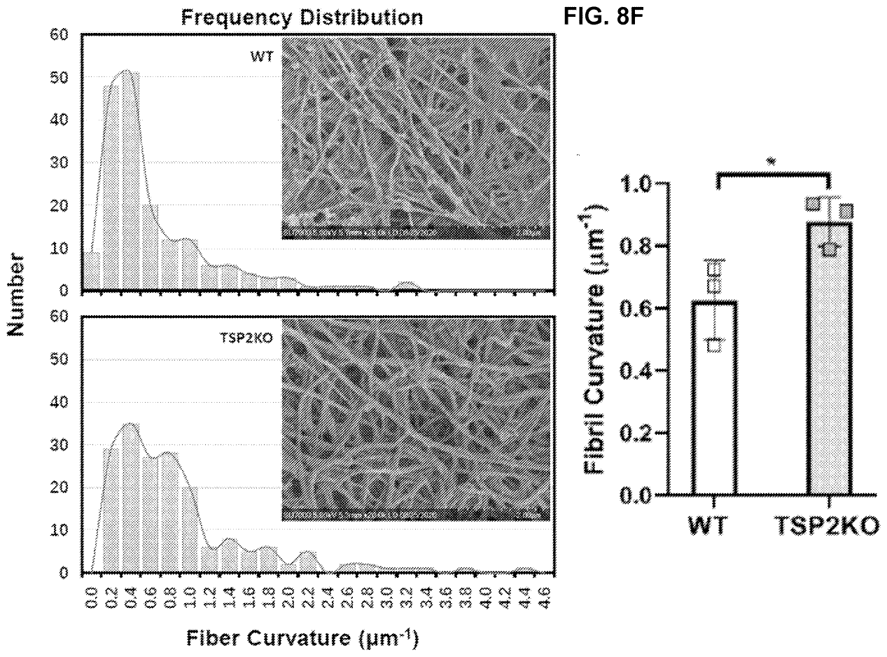

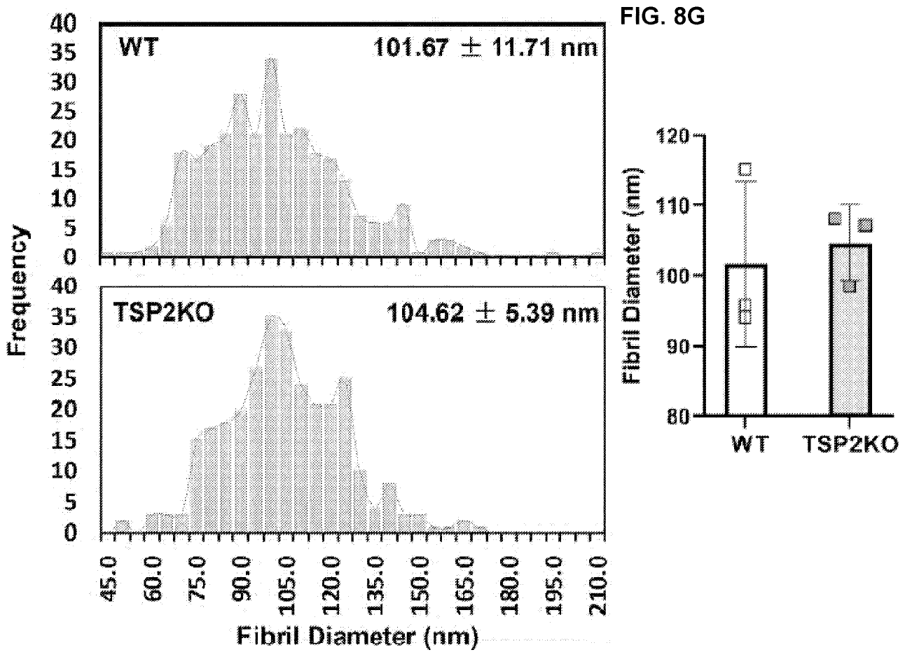

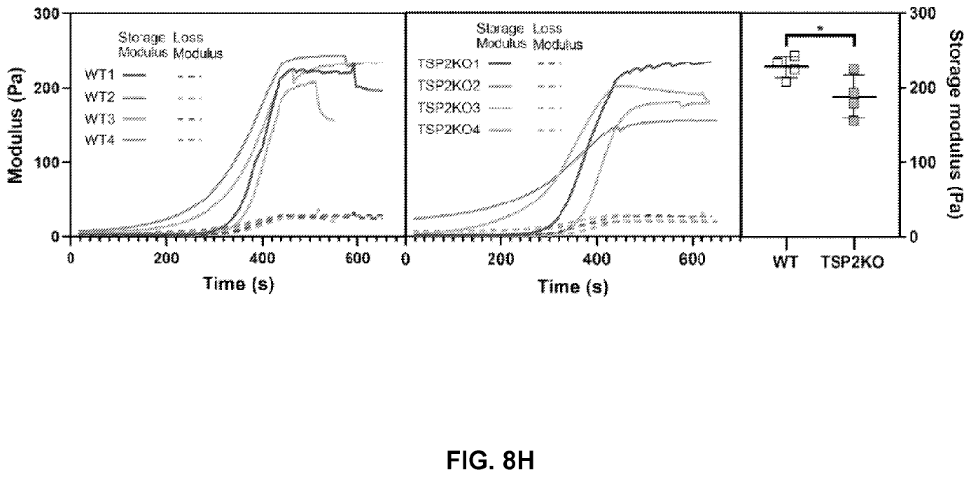

Preparation of bone-derived ECM hydrogels was performed according to the following steps: (i) mice bones were isolated from WT and TSP2KO mice; (ii) the bones were sectioned to produce fragments; (iii) the granules were demineralized under agitation at 300 rpm in 0.5 N HCl (25 ml/g bone) at RT for 24 h; (iv) the demineralized bone matrix (DBM) was decellularized in 0.05% trypsin and 0.02% EDTA at 37° C. and 5% CO₂ under continuous agitation for 24 h; (v) decellularized matrix (ECM) was rinsed in PBS supplemented with 1% w/v penicillin/streptomycin under continuous agitation for 24 h at 4° C. to remove residual cellular material. The ECM was digested by pepsin for 96 h, stopped digesting and put at 37° C. for 1 h, the pre-gel changed into the hydrogel. Photographs of samples from these processing stages are shown in FIG. 8A. H&E staining of sections from the HCl and Trypsin/EDTA steps show that the cells are removed step by step and Trichrome staining reveals that the collagen stays in the bone ECM (FIG. 8B).

The cell's DNA content was tested by the DNeasy Kit and NanoDrop and a significant difference was detected after decellularization for both WT and TSP2KO samples (***p<0.001, n=6) (FIG. 8C). Western Blot proved TSP-2 was present in the WT mice bone and absent within the TSP2KO mice bone (FIG. 8D). The proteins in each step of decellularizing mouse bone were tested by SDS-PAGE (FIG. 8E).

Next, the curvature of self-assembled collagen fibers in the bone-derived ECM hydrogels was assessed. 6 mg/ml pre-gel was put into the 48-well culture plate at 37° C. for 1 h. After that, the gels were fixed by 2.5% glutaraldehyde. Then an ethanol solution at step gradient concentrations (i.e., 30, 50, 70, 90 and 100%) was used to dehydrate the specimens for about 15 minutes per gradient concentration. After drying, the bone-derived ECM hydrogels (6 mg/ml) were observed by SEM (FIG. 8F). In both WT and TSP2KO hydrogels, collagen self-assembles into nanofibers. Analysis of the diameter of fibrils of the WT and TSP2KO hydrogels indicated that their diameter had no significant difference. N=300 fibrils in each group, 100 fibrils per sample, n=3 samples in each group, p>0.05 (FIG. 8G). The average curvature of TSP2KO fibrils is higher than that of WT fibrils. N=180 fibrils in each group, 60 fibrils per sample, n=3 samples in each group, *p<0.05 (FIG. 8F).

Modulus analysis of the bone-derived ECM hydrogels was performed via rheometer testing of storage modulus and loss modulus for WT and TSP2KO ECM hydrogels at 6 mg/ml (FIG. 8H). The storage modulus of WT group is significantly higher than that of TSP2KO group (*p<0.05 (n=4)).

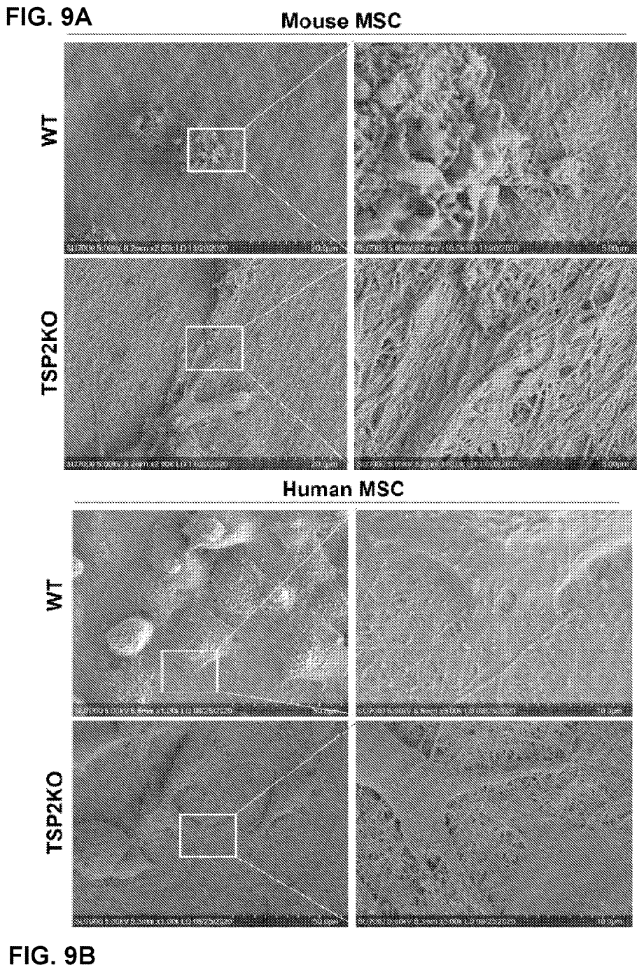

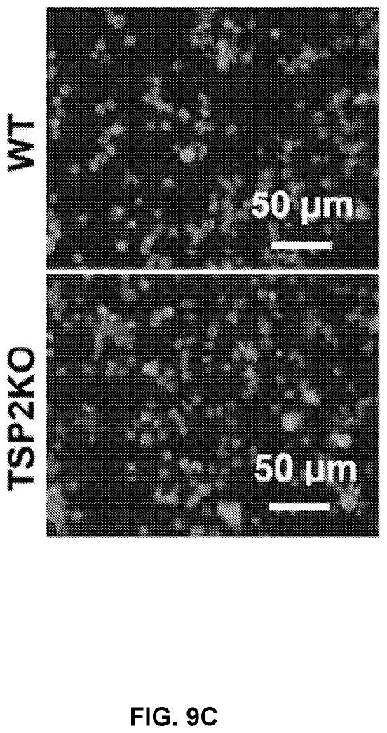

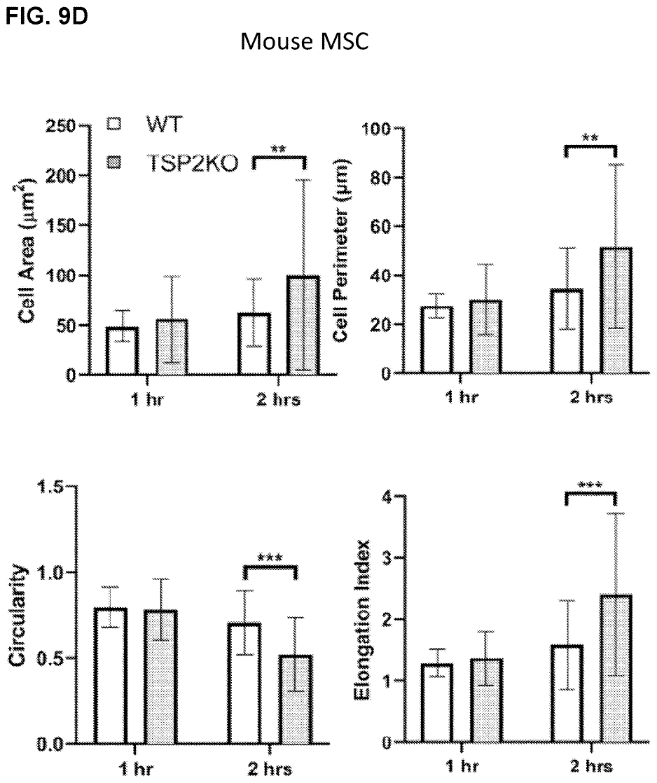

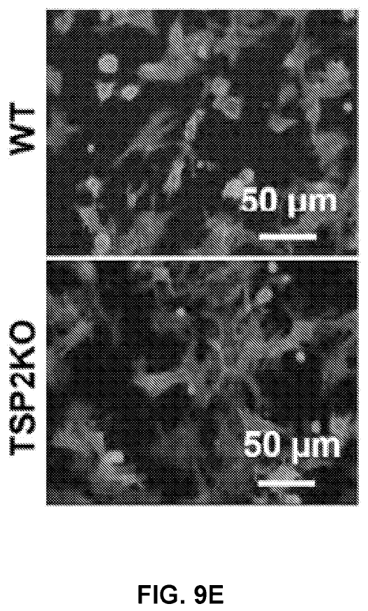

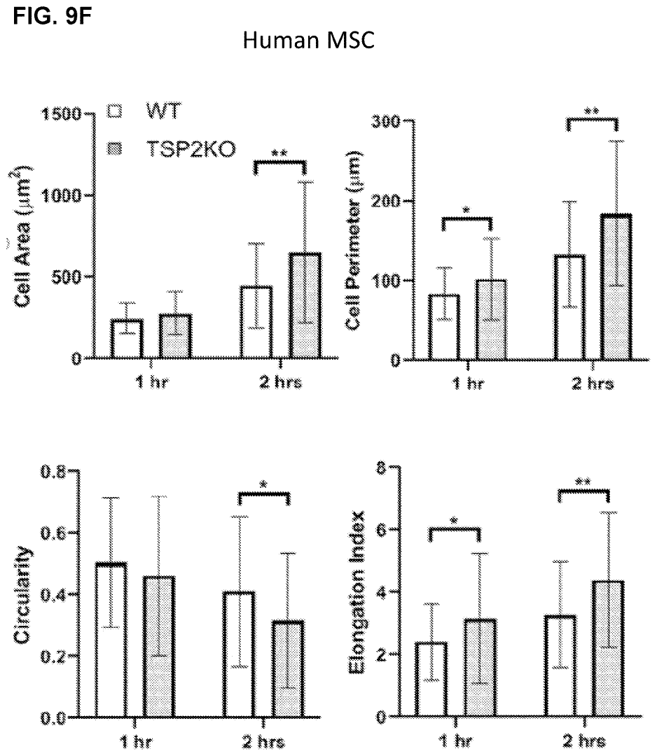

Next, a grouped attachment assay of mouse and human MSCs on bone-derived ECM hydrogels was performed. 1×10⁵ mouse MSCs or 1×10⁵ human MSCs were seeded on 6 mg/ml WT or TSP2KO hydrogel respectively, cultured for 2 hours in 48-well culture plate, and then the hydrogels were rinsed by PBS to remove the unattached cells. After that, the specimens were fixed by 2.5% glutaraldehyde. The specimens were dehydrated for about 15 minutes respectively at gradient concentrations (i.e., 30, 50, 70, 90 and 100%). After drying, SEM was used to examine the specimens for the coated Iridium. Pseudocolor was employed to visualize the interaction of the ECM matrix and the MSC (FIGS. 9A-9B). The results indicate that the TSP2KO derived gel provided a structure that allowed for MSCs integration into the gel whereas the WT derived gel did not.

Next, 20 K mouse MSCs or 20 K human MSCs were seeded on WT or TSP2KO hydrogel, cultured in 96-well culture plate, and imaged at 1 hr and 2 hrs. Representative images of MSCs cultured for 2 hours and stained with phalloidin (cytoskeleton, red) and DAPI (nucleus, blue) are shown in FIG. 9C and FIG. 9E. The images were quantified for the mouse (FIG. 9D) and human (FIG. 9F) MSCs for the area, perimeter, circularity, and elongation index (one-way ANOVA with post hoc Tukey HSD test, n=50 cells, *p<0.05, **p<0.01, ***p<0.001).

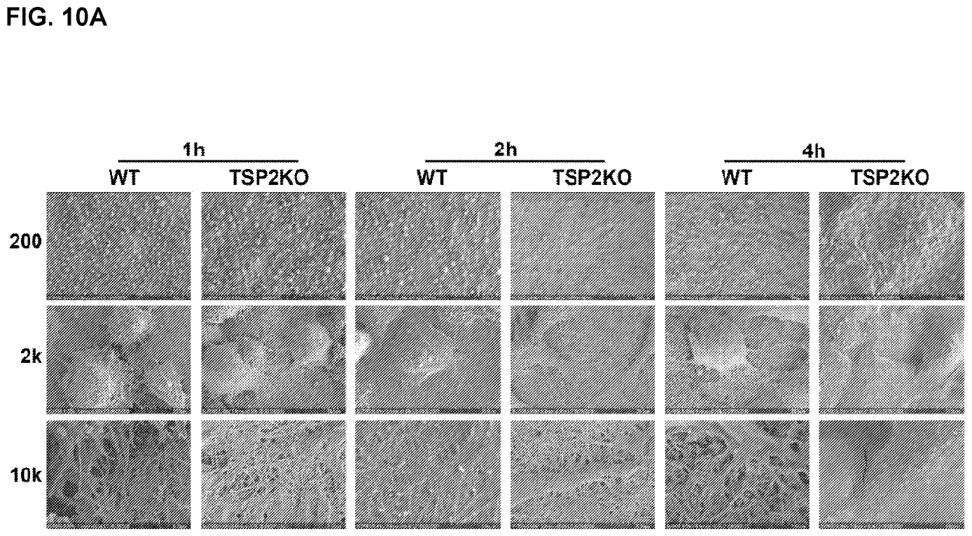

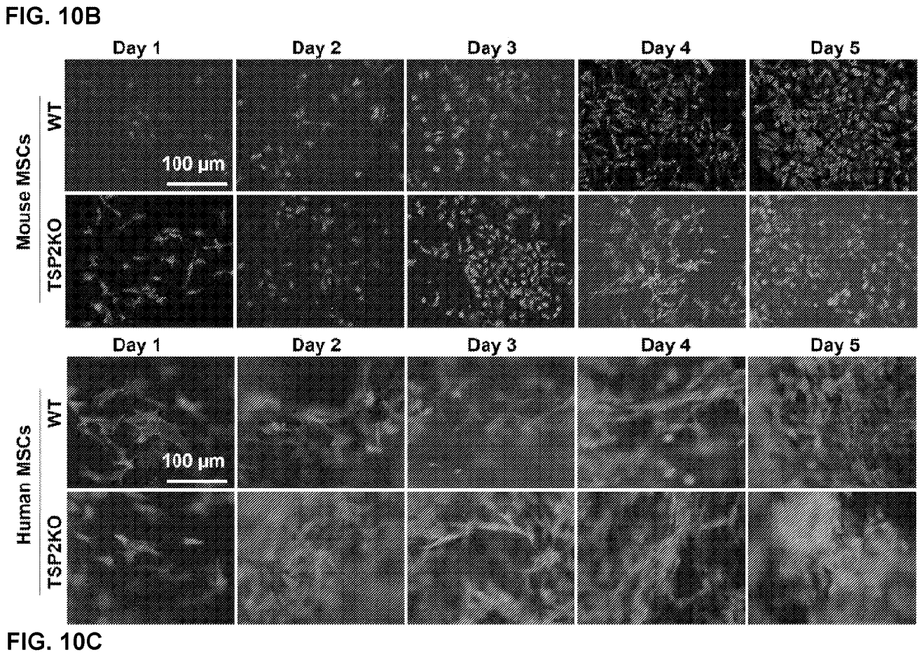

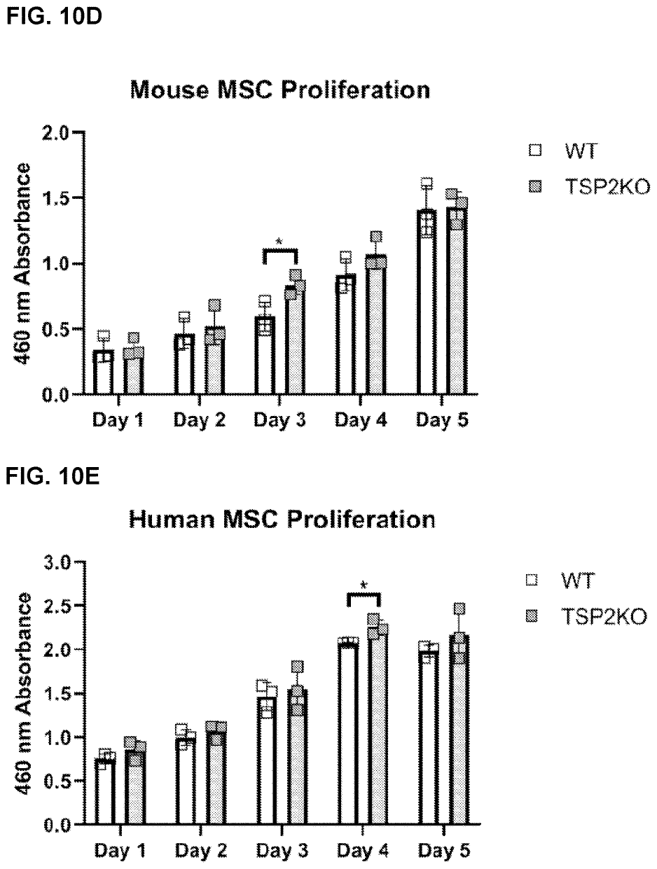

Next, MSC attachment and proliferation on the WT and TSP2KO bone-derived ECM hydrogels were assessed. SEM images were employed to assess the human MSCs attachment for the two hydrogel types (FIG. 10A). Mouse MSCs (FIG. 10B) and human MSCs (FIG. 10C) were cultured for 5 days on either WT or TSP2KO derived hydrogels. The nuclei were stained with DAPI (blue), and the cytoskeleton was stained with rhodamine-phalloidin (red). Mouse MSCs (FIG. 10D) and human MSCs (FIG. 10E) proliferation were analyzed every day for 5 days using CCK-8. Analysis of the proliferation assay indicated that TSP2KO hydrogel could promote mouse and human MSCs proliferation (*p<0.05, n=3).

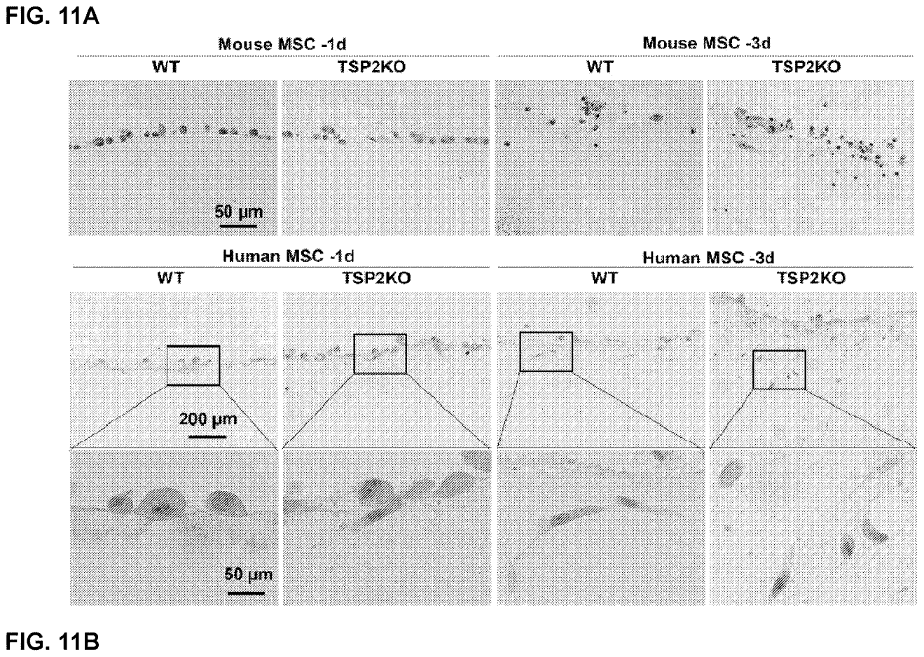

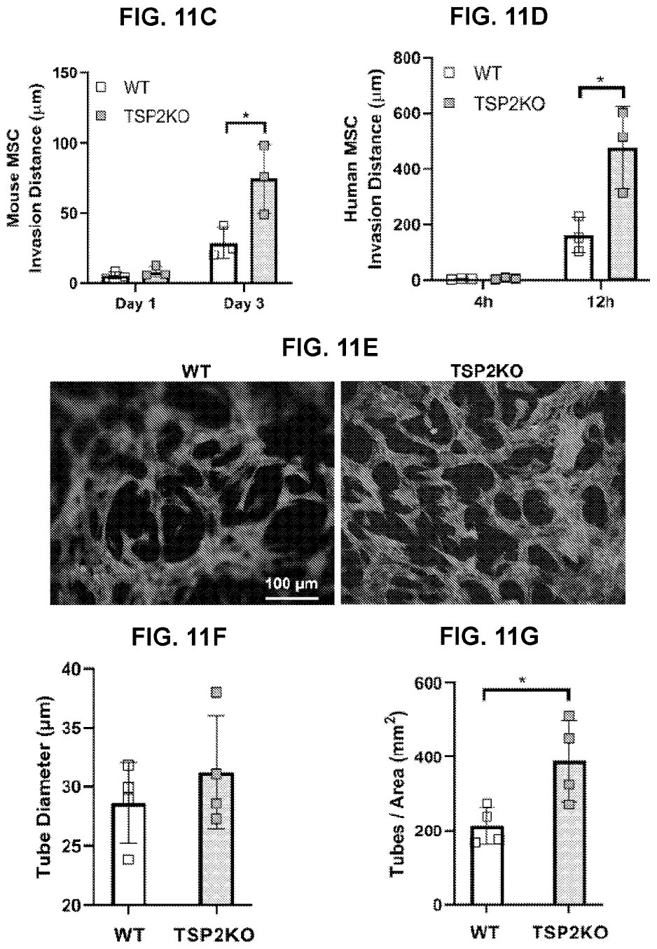

Next, mouse and human MSC invasion on the WT and TSP2KO bone-derived ECM hydrogels was assessed. A transwell was coated with pre-gel and incubated at 37° C. for 30 min. 50 K mouse MSCs (FIG. 11A) or 50 K human MSCs (FIG. 11B) were seeded on each gel and cultured for 1 and 3 days. The samples were washed to remove the unattached cells and then were fixed with z-fix overnight. A 70% ethanol solution was used to exchange the z-fix and the samples were embedded in paraffin for sectioning. The sections were stained with H&E according to standard protocols. Mouse MSC (FIG. 11C) and human MSC (FIG. 11D) invasion distance were calculated by Image J and it was determined that each group had 72 cells at 24 cells per sample. TSP2KO hydrogel showed a significant enhancement in MSCs invasion observed on day 3 (*p<0.05, n=3) compared to WT hydrogel.

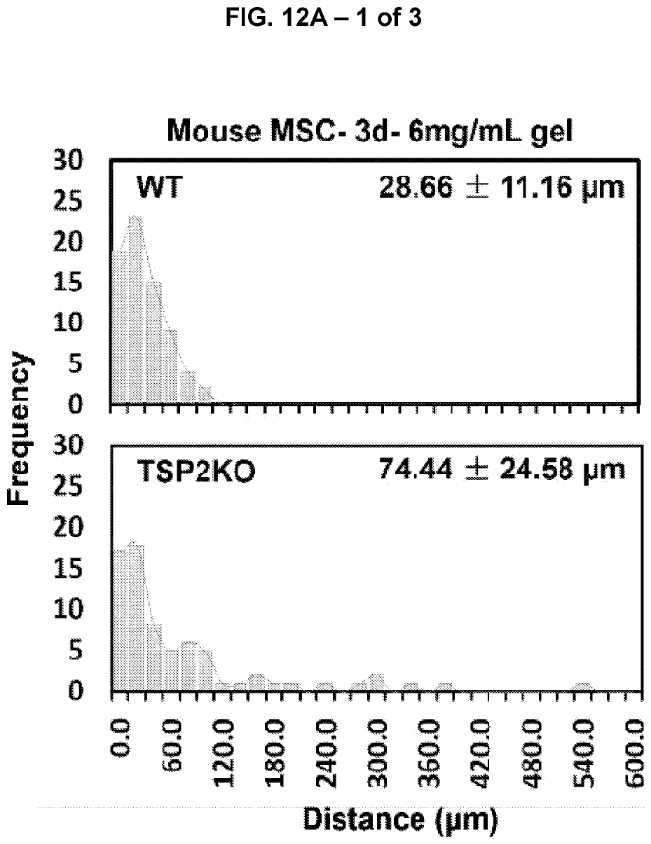

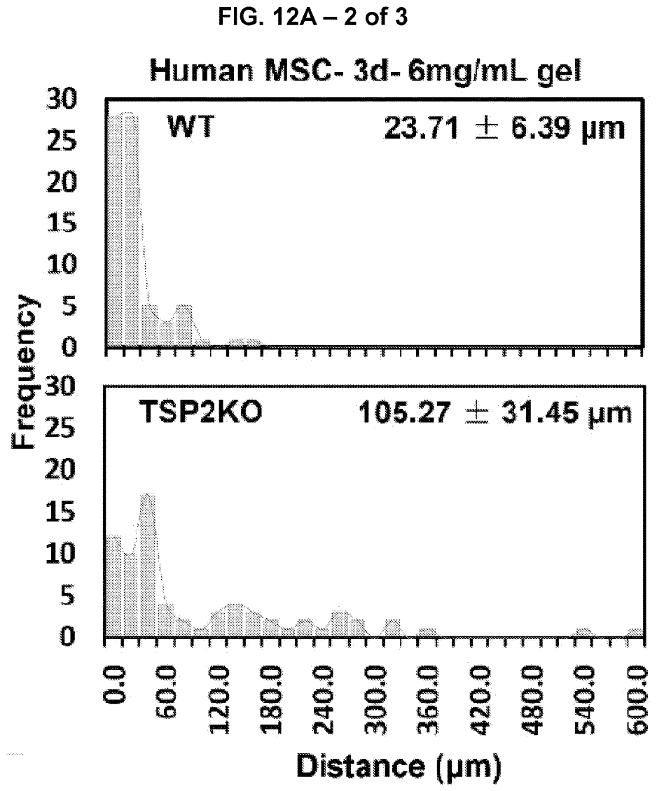

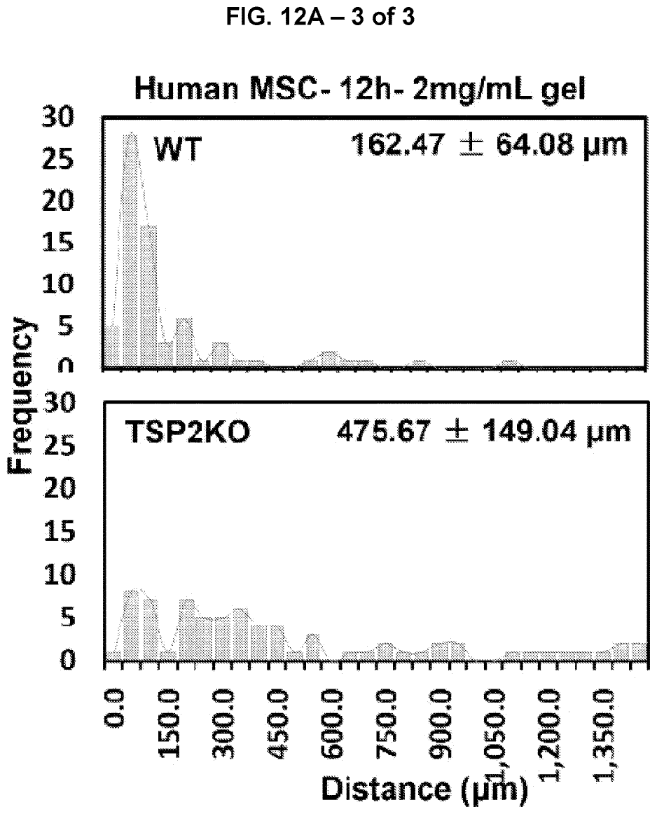

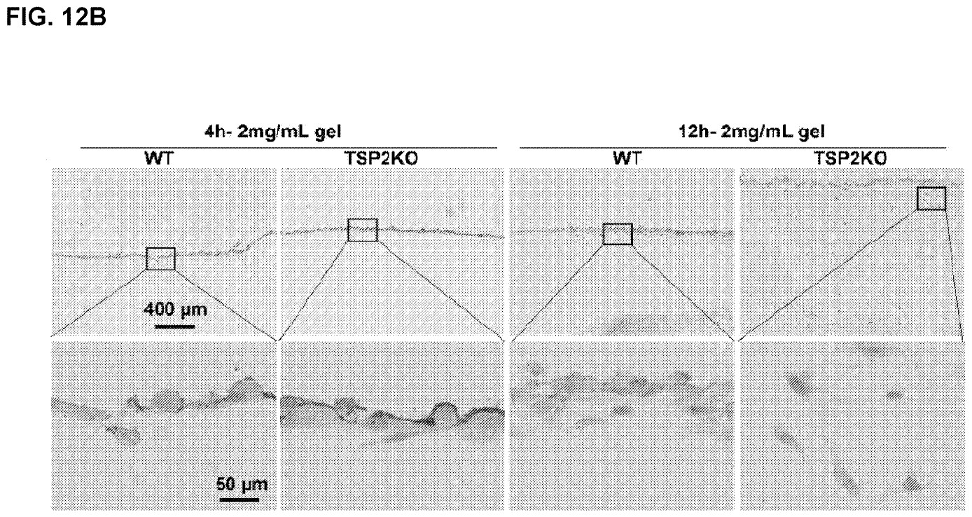

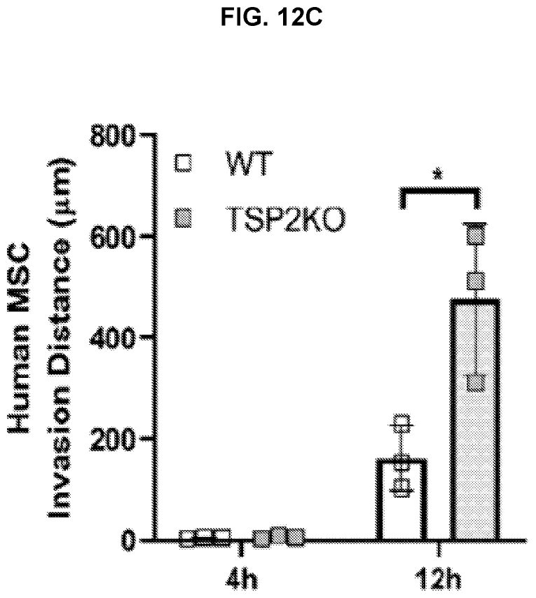

Mouse and human MSCs invasion was also assessed on 2 mg/mL bone-derived ECM hydrogels. A transwell was coated with pre-gel and incubated at 37° C. for 30 min. 50 k human MSCs were seeded on each 2 mg/ml gel and cultured for 4 and 12 h, respectively. The samples were washed, fixed and stained with H&E (FIG. 12B). The frequency distribution of mouse MSC and human MSC invasion distance in the hydrogels was indicated by the H&E staining (FIG. 12A). The TSP2KO hydrogel supported a significantly enhanced human MSC invasion in 2 mg/mL hydrogel observed at 12 hours (*p<0.05, n=3) (FIG. 12C).

Next, HUVEC tube formation on the WT and TSP2KO bone-derived ECM hydrogels was assessed. 10 K HUVECs were seeded on WT and TSP2KO hydrogels and cultured for 24 hours. The nuclei were stained with DAPI (blue), and the cytoskeleton was stained with rhodamine-phalloidin (red) (FIG. 11E). The fluorescence imaging indicated that the diameter of the formed tubes had no significant difference between WT and TSP2KO groups (p>0.05, n=4) (FIG. 11F), but the TSP2KO hydrogel produced significantly more tubes than the WT hydrogel (*p<0.05, n=4) (FIG. 11G).

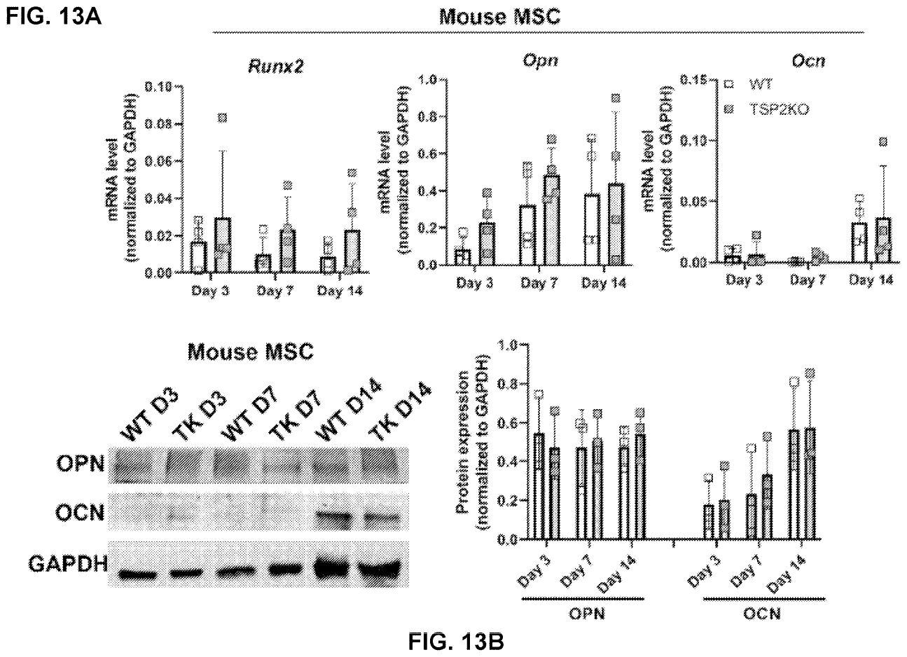

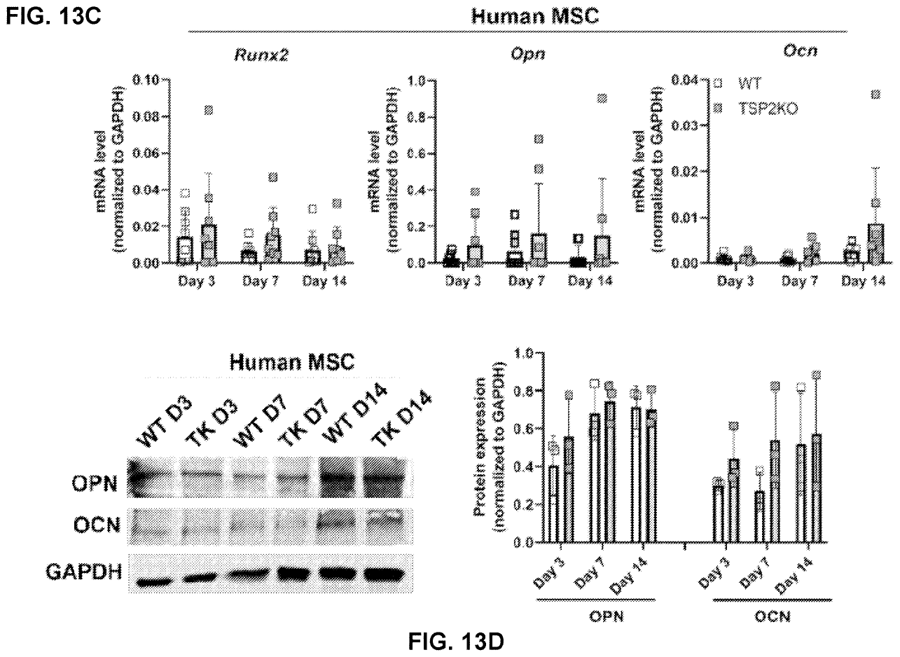

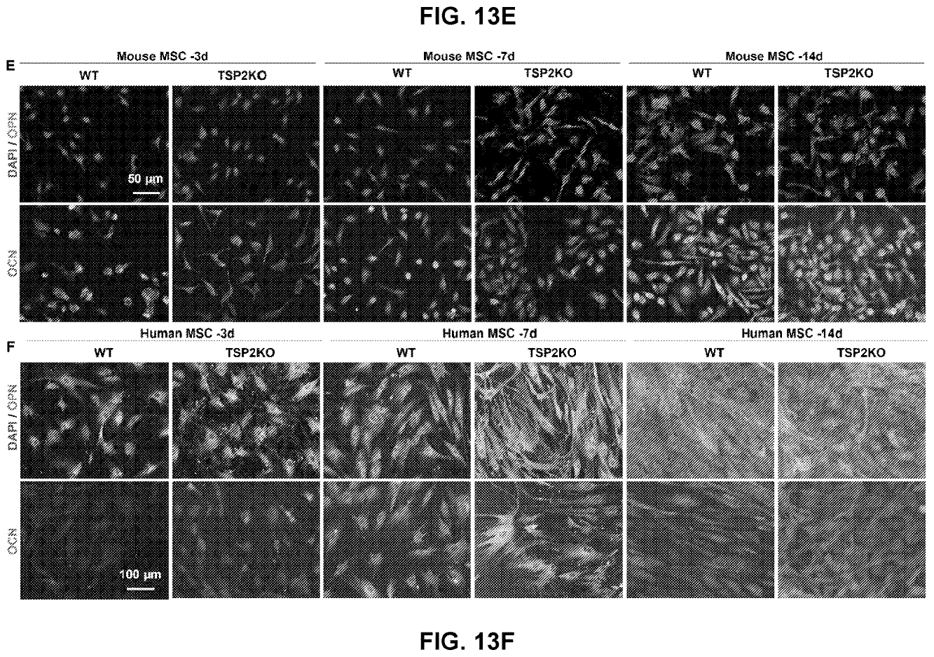

Osteogenic differentiation of mouse and human MSCs on the WT and TSP2KO bone-derived ECM hydrogels was determined by assessing gene expression via qRT-PCR, which indicated no significant difference between the MSCs cultured on WT or TSP2KO hydrogels (p>0.05, n=4) (FIG. 13A and FIG. 13C). Rather, both WT and TSP2KO hydrogels were beneficial for osteogenic differentiation of MSCs, as demonstrated by Western blots (FIG. 13B and FIG. 13D) and fluorescence imaging (FIG. 13E and FIG. 13F) of osteopontin (OPN) and osteocalcin (OCN) expression in lysates collected 3-, 7- and 14-day post-culturing on WT and TSP2KO hydrogels. The RT-qPCR primers are shown in Table 2.

TABLE 2
RT-qPCR primers

| Gene | Primers |
|---|---|
| Human | 5′-CGGAATGCCTCTGCTGTTAT-3′ |
| Runx2 | (F) (SEQ ID NO: 15) |
| | 5′-TGTGAAGACGGTTATGGTCAAG-3′ |
| | (R) (SEQ ID NO: 16) |
| Human | 5′-TCTGATGAACTGGTCACTGATTT-3′ |
| OPN | (F) (SEQ ID NO: 17) |
| | 5′-CTCGGCCATCATATGTGTCTAC-3′ |
| | (R) (SEQ ID NO: 18) |
| Human | 5′-TGCAGAGTCCAGCAAAGG-3′ |
| OCN | (F) (SEQ ID NO: 19) |
| | 5′-CCCAGCCATTGATACAGGTAG-3′ |
| | (R) (SEQ ID NO: 20) |
| Human | 5′-GGTGTGAACCATGAGAAGTATGA-3′ |
| Gapdh | (F) (SEQ ID NO: 21) |
| | 5′-GAGTCCTTCCACGATACCAAAG-3′ |
| | (R) (SEQ ID NO: 22) |
| Mouse | 5′-GCCACTTACCACAGAGCTATT-3′ |
| Runx2 | (F) (SEQ ID NO: 23) |
| | 5′-GAGGCGATCAGAGAACAAACT-3′ |
| | (R) (SEQ ID NO: 24) |
| Mouse | 5′-ACGACGATGATGACGATGATG-3′ |
| OPN | (F) (SEQ ID NO: 25) |
| | 5′-GACTCATCCGAATGGTGAGATT-3′ |
| | (R) (SEQ ID NO: 26) |
| Mouse | 5′-GTGAGCTTAACCCTGCTTGT-3′ |
| OCN | (F) (SEQ ID NO: 27) |
| | 5′-AGTGATACCGTAGATGCGTTTG-3′ |
| | (R) (SEQ ID NO: 28) |
| Mouse | 5′-TTCACCACCATGGAGAAGGC-3′ |
| Gapdh | (F) (SEQ ID NO: 29) |
| | 5′-GGCATGGACTGTGGTCATGA-3′ |
| | (R) (SEQ ID NO: 30) |

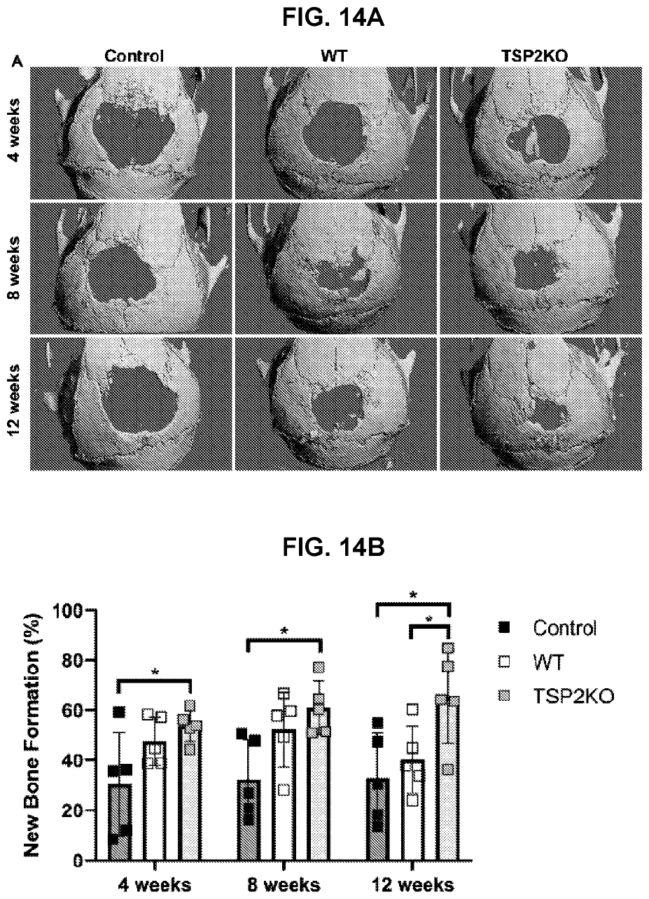

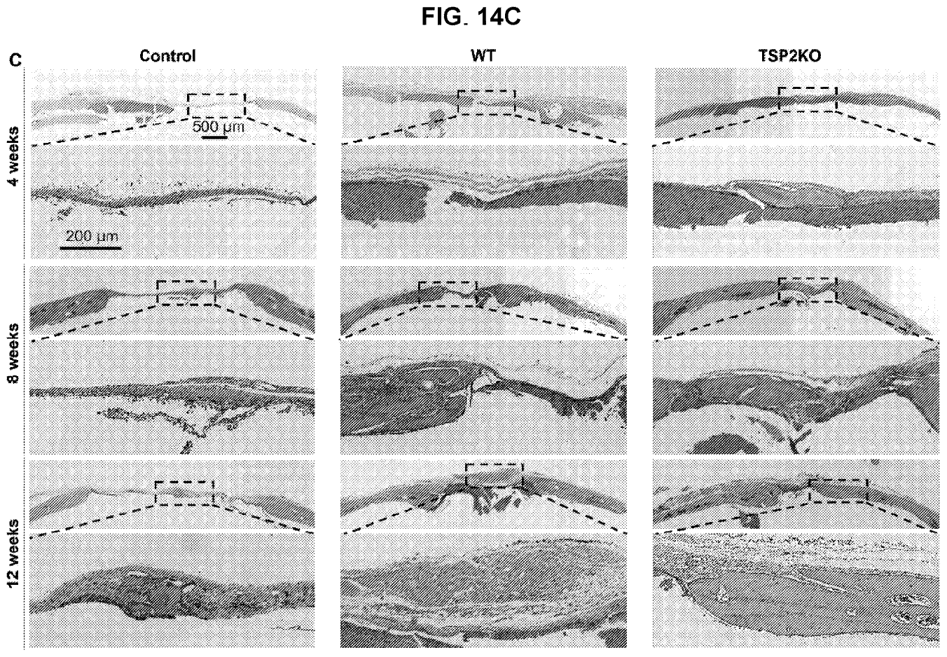

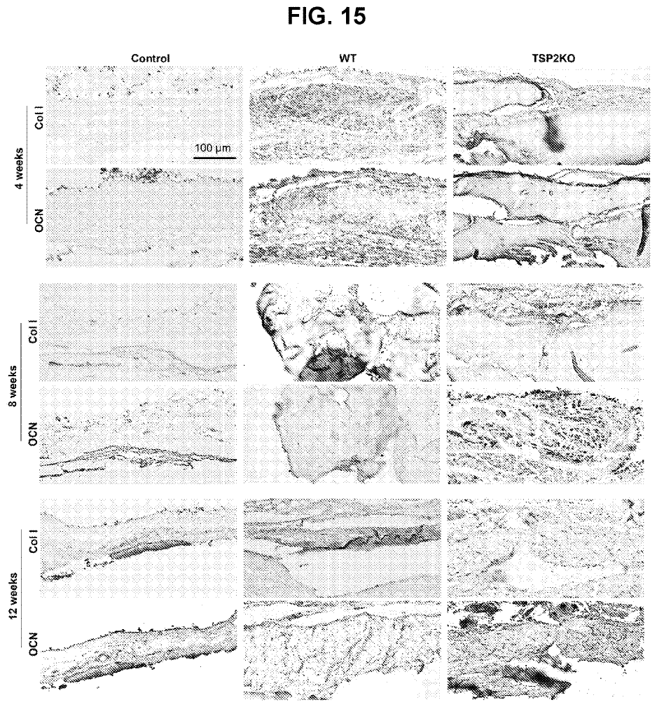

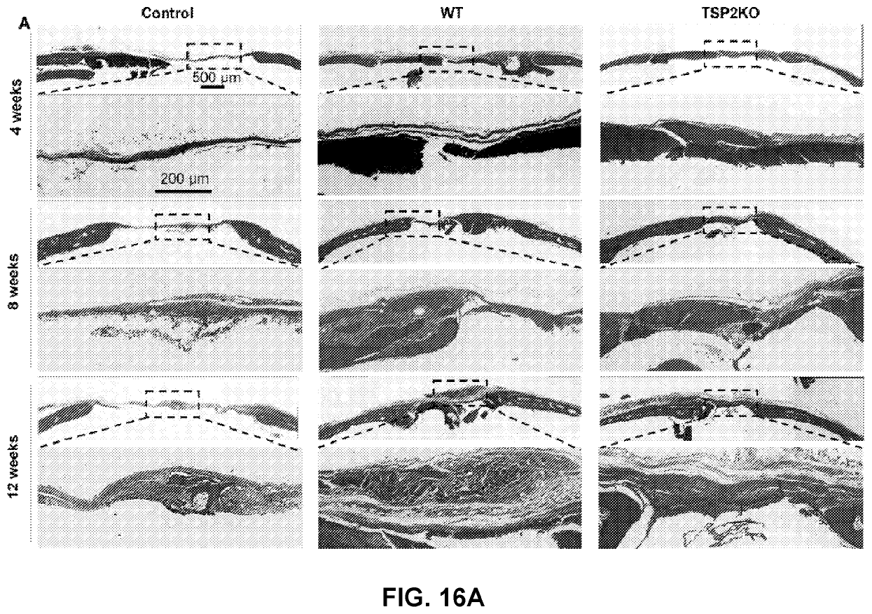

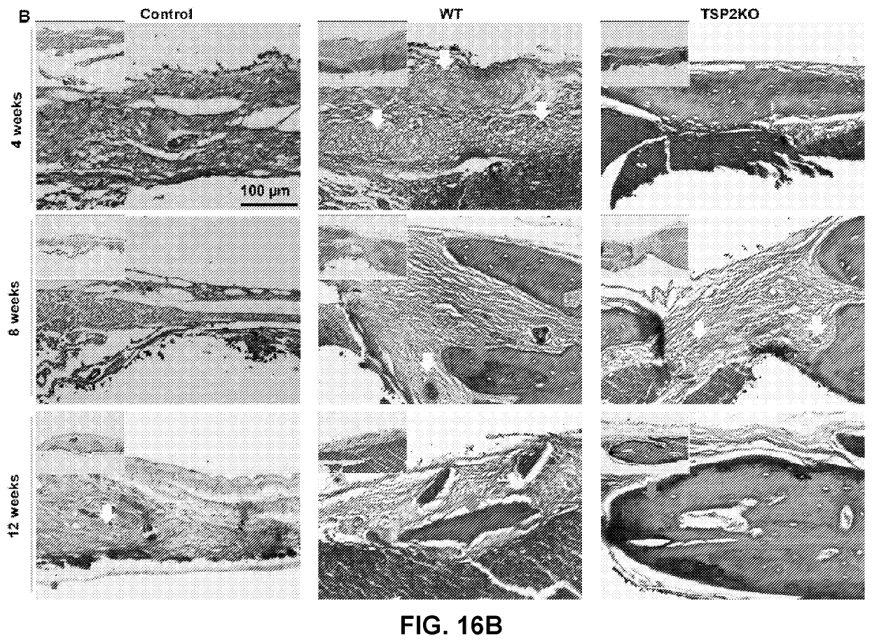

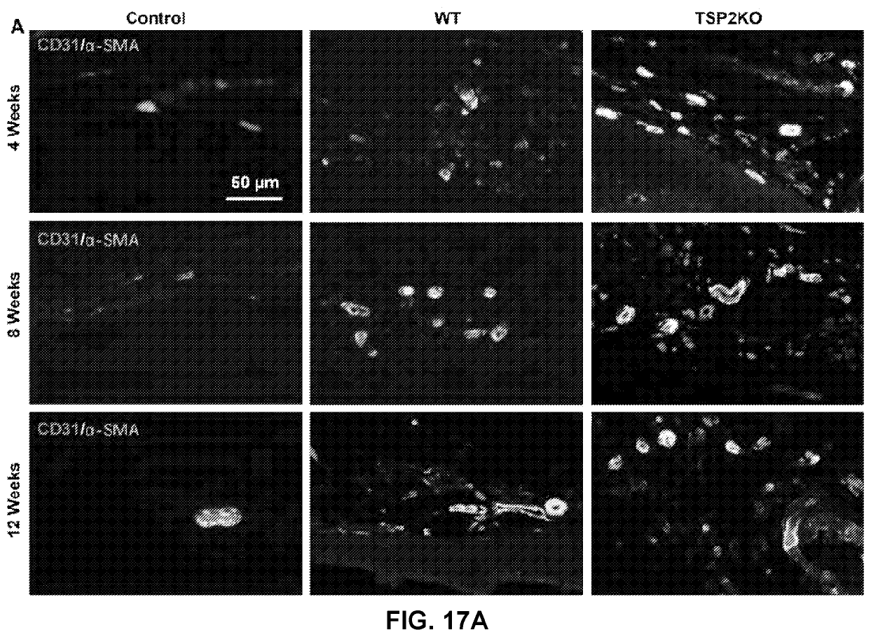

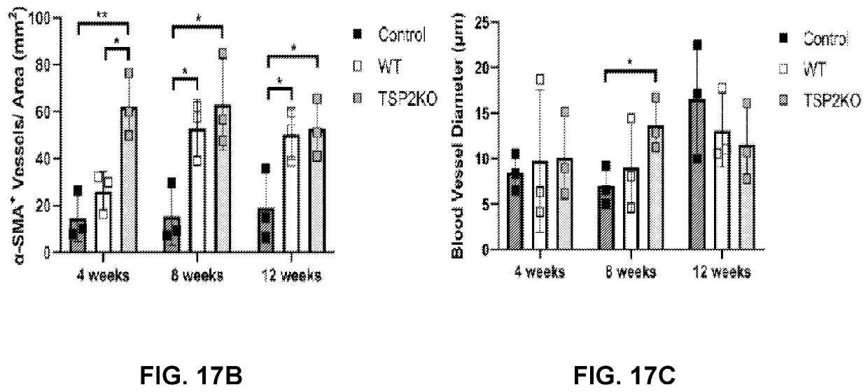

Next, in vivo wound repair was assessed in the calvarial defect mouse model treated with bone-derived ECM hydrogels. WT or TSP2KO hydrogel was implanted in C57BL6/J mice with critical size defect (5 mm diameter defect). Control mice did not receive hydrogel. Micro-CT scans of the wound site 4, 8, and 12 weeks after implantation (FIG. 14A) revealed that the percentage of the new bone formation for TSP2KO hydrogel was significantly improved compared to that in the non-implanted control group at weeks 4, 8 and 12 post implantation (*p<0.05, n=5), and compared to the WT hydrogel group at week 12 post implantation (*p<0.05, n=5) (FIG. 14A and FIG. 14B). The newly formed tissues were further analyzed by H&E staining (FIG. 14C) as well as immunochemistry staining for Col I and OCN to evaluate the ECM deposition and the regenerated bone tissue (FIG. 15). Trichrome staining analysis of the newly formed bone and cartilage was also performed (FIG. 16A), as well as Saffranin O/Fast Green staining analysis of the chondrocytes in the newly formed tissues (FIG. 16B). Immunofluorescence was used to analyze the blood vessels in the newly formed tissues. Immunofluorescence staining for PECAM-1 (CD31, red) and alpha smooth muscle actin (α-SMA, green) shows microvascular endothelium in adventitia and outer vessel wall (FIG. 17A). The blood vessels were quantitatively analyzed via Image J. The analysis indicated that there were more blood vessels present in the newly formed tissue in the TSP2KO hydrogel as compared to the WT hydrogel post-surgery 4 weeks (*p<0.05, n=3) (FIG. 17B).

The diameter of the blood vessels was quantitatively analyzed via Image J. The analysis indicated that there was no significant difference between WT and TSPKO group (p>0.05, n=3) (FIG. 17C).

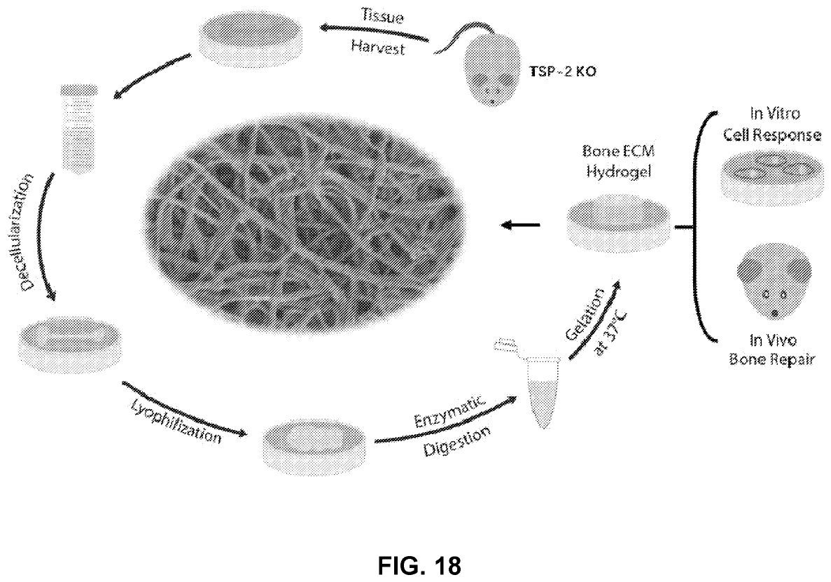

A summary of the preparation process for bone-derived ECM hydrogel and in vivo and in vitro testing thereof is show in FIG. 18.

### Example 3: Decellularized ECM Hydrogels for Cartilage Repair

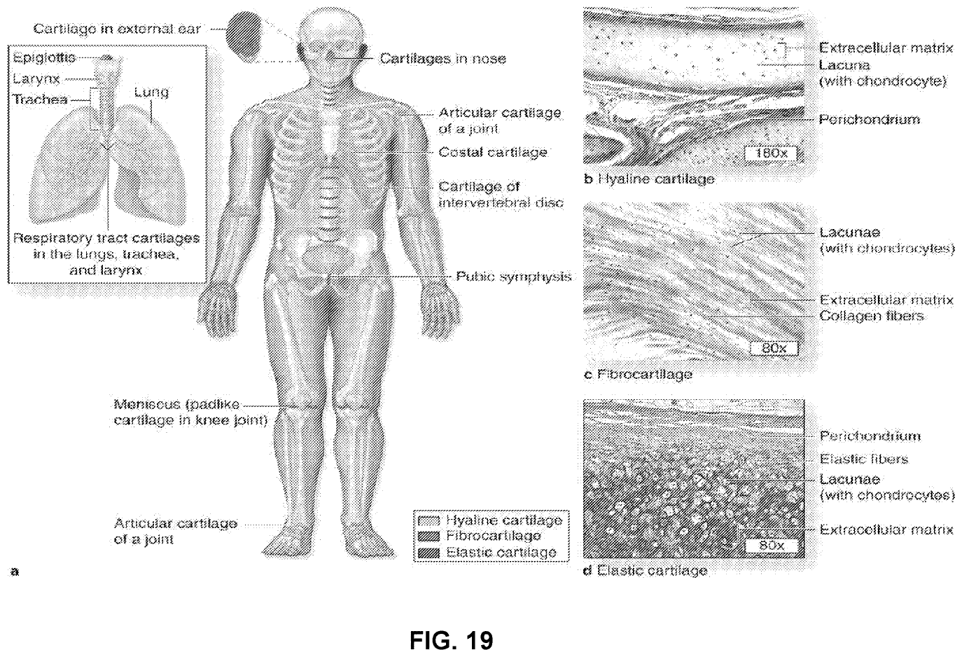

Cartilage is roughly classified into three types: hyaline cartilage, fibrocartilage and elastic cartilage. All three possess common features (e.g. chondrocytes inside lacuna chambers). However, they have different ECM composition and organization. Hyaline cartilage is also known as articular cartilage since it often appears in joints. It is smooth in appearance, yet flexible and strong. Fibrocartilage is most commonly found in intervertebral disks and knee meniscus. It is the strongest and least flexible of the three types—the ECM is stiffer because of the presence of type I collagen in addition to type II commonly found in other cartilage types. Elastic cartilage is found in the ear and larynx. It is the most flexible and has elastins and type II collagen as major ECM components. FIG. 19 shows distribution and structural differences of different cartilage types. Major components of all cartilage types include collagen type II, proteoglycans and glycosaminoglycans (GAGs). Since all contain adequate amount of proteins, hydrogels can be derived from the three types of cartilage for comparison of their properties.

Decellularized ECM (dECM) hydrogel is a novel biomaterial system that will be explored in this project. It maintains native ECM components while removing immunogenic materials by an extensive decellularization process. Moreover, inherent ECM molecules such as collagen, GAG, laminin and growth factors are preserved with a careful approach. Compared to synthetic materials, dECM hydrogel is more biocompatible, biodegradable, and can promote tissue regeneration. Cartilage dECM scaffolds have been fabricated, as reported in recent studies, with different approaches. dECM can be digested with acetic acid and crushed into an ice bath to fabricate a gel-like bioink. Alternatively, dECM can be digested with HCl, neutralized, and crosslinked at 37° C. to form a hydrogel. Similar to hydrogels derived from other tissues, cartilage dECM scaffolds could be loaded with external components and induce regeneration in vivo.

Here, the role of matricellular protein TSP2 will be explored in cartilage ECM. TSP2 is known to participate in various molecular pathways. Its role in promoting chondrogenesis and inhibiting bone formation has been suggested in previous studies. In a mouse model, TSP2 is secreted by bone marrow mesenchymal stem cells (bmMSCs) and retroactively inhibits bmMSC proliferation. Recent studies have reported the supportive role of TSP2 in chondrogenesis by signaling to progenitor cells, which induces their differentiation via a range of pathways. A TSP2-knock-out (TSP2-KO, or TKO) genotype is associated with increased endosteal and corticol bone formation because of increased osteoblast differentiation. Despite this enhancement, TKO mice exhibit reduced osteoclast activity and chondrogenic marker expression. This is accompanied by more bone and less cartilage formation post fracture. It has been suggested that TKO induces vascularization in the fracture niche, inhibiting Hypoxia-Inducible Factor (HIF) while promoting an oxygen-rich environment that favors progenitor differentiation into osteoblasts.

TSP2 is also known to have prominent effect in ECM structure and organization. In ECM from different tissues, the TKO genotype is associated with increased proteolytic activity by matrix metalloproteinases (MMPs), increased tissue flexibility, and irregular organization of collagen fibrils. As a result, TKO mice have softer, more fragile ECM and improved wound healing. In contrast, elevated TSP2 is reported to be associated with delayed wound healing, especially in diabetic mice. dECM hydrogel derived from TKO skin has shown efficacy in a skin biopsy healing model. TKO's regenerative advantages come, in part, from TSP2's antiangiogenic role—the KO genotype promotes vascularization and ECM remodeling during wound healing. In avascular cartilage tissues, the pro-angiogenic effect may be less important, but differences in ECM structure may still promote tissue repair.

While dECM hydrogel itself may already provide increased regeneration, modification of hydrogel with external agents is a common approach to improve its potency. Agents of modification could include nano/microparticles, cells, growth factors, exosomes, etc. For example, Poly(lactic-co-glycolic acid) (PLGA) nanoparticles and microspheres act as potent loading agents because of their tunability, biodegradability, non-toxicity and the capability of loading growth factors for controlled release. Previous studies have demonstrated feasibility of fabricating PLGA microspheres (for prolonged release due to their larger size) loaded with Bone Morphogenetic Protein 2 (BMP-2) and Transforming Growth Factor β (TGFβ) for bone engineering. With potential of being incorporated into a naturally-derived dECM network, there exists possibility of further enhancing cartilage regeneration by encapsulating growth factor-loaded PLGA particles inside hydrogels.

The project consists of three parts involving the fabrication, characterization and validation of a novel dECM biomaterial that has therapeutic potential for the repair of osteochondral defects. A cartilage dECM enriched hydrogel system is fabricated and characterized, with cross comparison between wild-type (WT) and TKO cartilage dECM. Then, the interaction between hydrogel and cells will be explored. In addition, modification of hydrogel function with external agents will be tested in vitro. Finally, cartilage dECM-derived hydrogels will be introduced to in vivo cartilage defect models to validate therapeutic efficacy of the novel biomaterial.

Fabrication and Validation of WT/TKO Cartilage dECM-Derived Hydrogels

This is the foundation of the project by making and characterizing dECM hydrogels from various cartilage tissues. Cartilages from WT and TKO animals are collected and decellularized, followed by solubilization and gelation. Successful decellularization is validated by DNA content and histology staining. Differences between WT and TKO cartilage dECM hydrogels are probed by rheometry, Scanning Electron Microscopy (SEM), porosity quantification and proteomics.

There are differences between WT and TKO cartilage ECM organization, which can be characterized. One part of the project develops a novel cell-free native cartilage ECM hydrogel. It also investigates the influence of TSP2 on cartilage ECM properties. This has been developed with elastic cartilage with the methodology applicable to other cartilages.

Development of Cartilage dECM-Derived Hydrogels

To optimize a biomaterial's therapeutic potential for osteochondral defects, it is reasonable to derive a scaffold from a comparable source. Native cartilage ECM has a unique composition and organization that likely plays a role in cell interaction and regenerative property. Cartilages are processed by decellularization, which is an established process to remove immunogenic cellular materials and DNA from source tissue while preserving cytoskeletal structures and important proteins. Hyaline, fibro and elastic cartilage have different ECM compositions, stiffness and elasticity, and which composition is optimal for hydrogel fabrication remains elusive. Therefore, it is necessary to fabricate and evaluate hydrogels from cartilage dECM from different tissue sources.

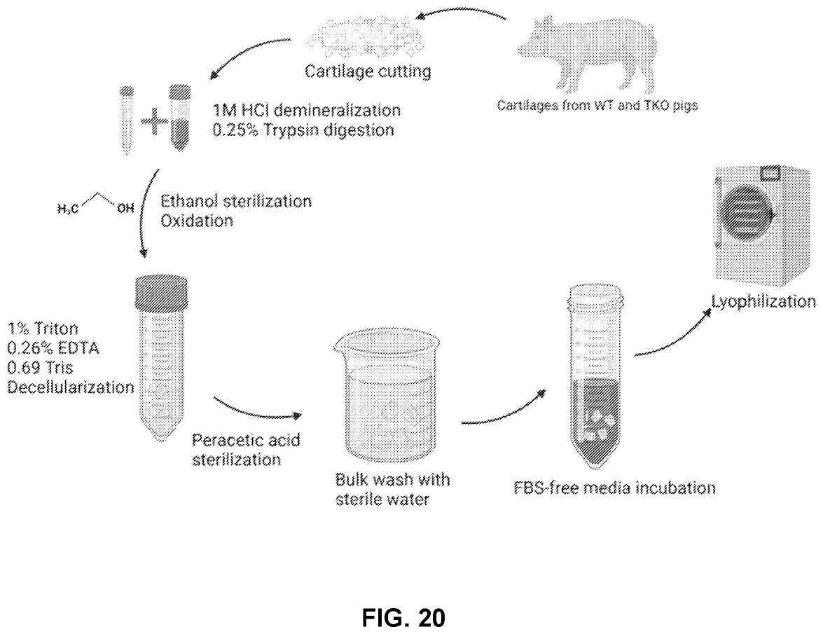

Protocols for ECM decellularization have been established for multiple tissue types. However, they need to be modified for decellularizing cartilage tissues, which possess osseous remnants and ECM that is difficult to penetrate with conventional approaches. The workflow of cartilage decellularization with representative images is shown in FIG. 20, which has been shown to work on WT and TKO pig ear elastic cartilages. Briefly, skin and fat remnants are first removed by hand and tissues are cut into approximately 1 cm×1 cm pieces. An optional demineralization step (for removing bone minerals) with 1M HCl precedes the initial processing with 0.25% trypsin-EDTA. After sterilization with ethanol, tissues undergo oxidation treatment with 3% hydrogen peroxide. Tissues are then decellularized in 1% Triton solution buffered with EDTA and Tris for 36 hours. Following decellularization, extensive sterilization and washing steps are performed with ethanol, peracetic acid and ddH₂O to ensure cleanness. Decellularized cartilages are finally incubated in FBS free culture media with 1% antibiotics overnight. The product is then lyophilized.

Workflow for dECM hydrogel fabrication is shown in FIG. 21. To formulate hydrogel, lyophilized tissues are first weighed and cryomilled. They are then solubilized with a 2 mg/ml pepsin in 0, 1M HCl solution with a concentration less or equal than 10 mg tissue/ml digestion solution. This gives a pregel solution that can be used for a variety of experiments. To neutralize the pregel, 10×PBS with 0.1 mg/ml phenol red indicator is used to buffer the pregel, and IM NaOH is used to titrate the pregel to quasi-neutral pH. Neutralized pregel is then incubated at 37° C. until gelation.

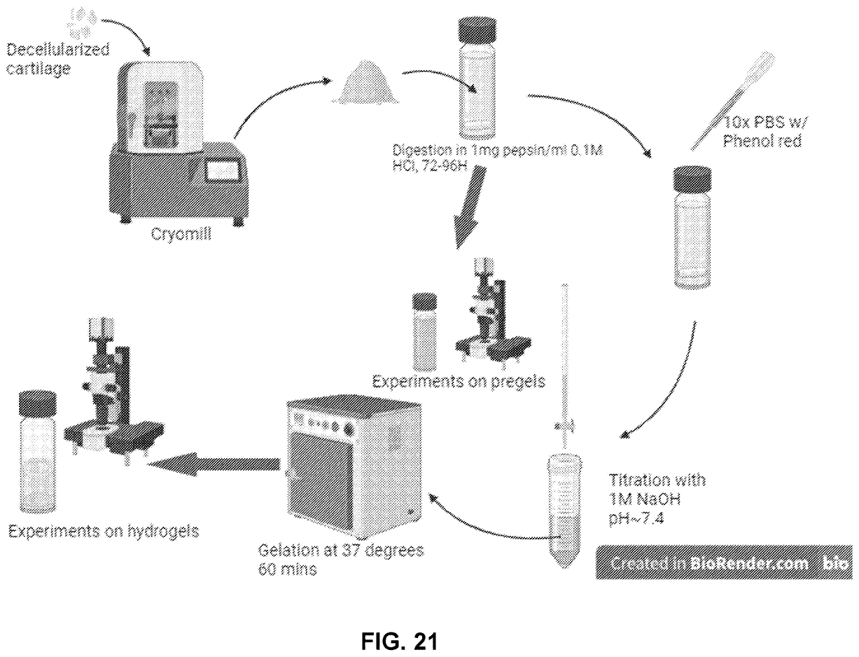

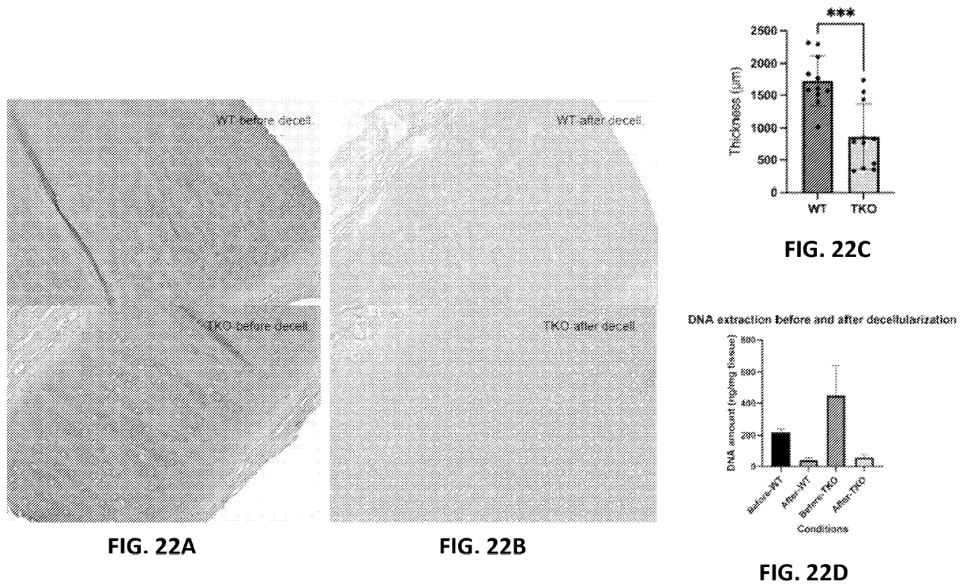

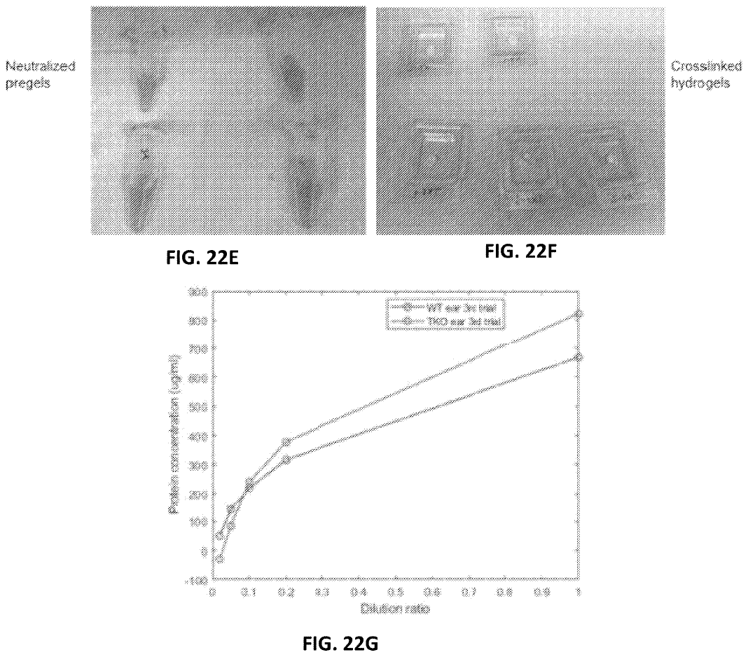

Preliminary experimental data with pig ear elastic cartilage are shown in FIG. 22A-22G. Pig ears have been decellularized with the described method with desirable gelation properties. Decellularization was successful as confirmed by DNA content and H&E staining, and hydrogels have been produced from both genotypes after digestion and gelation. Total protein quantification by BCA gives protein concentration values close to 1 mg/ml, which is lower than pregels prepared from skin with the same concentration for digestion, showing the uniqueness of cartilage tissue.

With experience from fabricating decellularized pig ear cartilage hydrogel, this approach was extrapolated to fabrication of a variety of hydrogels from different cartilage tissues. Validation process for other cartilage types are similar to that described above.

Characterization and Cross Comparison of WT/TKO Cartilage dECM Hydrogels

Although evidence suggests that TSP2 is absent from mature cartilage callus, it is has played a role in bmMSC differentiation and chondrogenesis. dECM from TKO animals is structurally and biomechanically different from WT. Property difference in cartilage dECM and dECM-derived hydrogels constitutes the basis of cell-ECM interaction and in vivo therapeutic potential in hydrogels from TKO cartilages. It is therefore essential to compare hydrogels from both genotypes to identify change in properties.

A series of characterization steps were carried out using pregel or hydrogel derived from WT and TKO pig cartilage tissues. Firstly, gelation dynamics are measured by turbidity spectrometry: neutralized pregels are diluted to 1 mg/ml and plated into a 96 well plate on ice. 1 mg/ml rat tail collagen I is used as positive control. A plate reader preheated to 37° C. is used to continuously read absorbance at 415 nm for 30 mins. The absorbance measurements are normalized and compared between genotypes (adapted from Jeong, Sang Young, et al. “Thrombospondin-2 secreted by human umbilical cord blood-derived mesenchymal stem cells promotes chondrogenic differentiation.” Stem Cells 31.10 (2013): 2136-2148).

For rheometry measurement using a TA Instrument HR-30 rheometer, gels are loaded onto a 20 mm sand-bottom parallel plate with a gap distance of 700 microns. Angular frequency and strain rate are fixed, while plate temperature is increased stepwise from 10 degrees to 37° C. with a hold time of 10-15 s for each step until gelation is observed. To probe viscoelastic behavior, temperature is fixed at 37° C., while the shear and strain rate are varied one at a time within toleration range of hydrogel. For both experiments, storage modulus, loss modulus and complex viscosity are the primary variables of interest as they change with varying temperature, shear and strain rate.

After gelation, dECM organization is measured with SEM. To setup for SEM, gels are fixed, dehydrated with ethanol gradient, and incubated in hexamethyldisilazane (HMDS) until completely dried. Hydrogels are then spuller-coated with 3 nm iridium and imaged with a Hitachi SU-70 SEM to visualize organization of ECM fibrils. In addition, gelated material is first lyophilized and then a cryomilled to convert to powder, which is analyzed with a Micrometrics ASAP 2460 for surface area and porosity.

To probe protein content differences, protein gel electrophoresis and proteomics are performed. For protein gel electrophoresis, the acidic pregel is first mixed with Laemmli loading buffer and denatured at 95 degrees. Denatured proteins from the two genotypes are then loaded at 50 μg/well inside a pre-cast polyacrylamide gel against a ladder reference. After electrophoresis, the gel is fixed, stained with Coomassie blue, and imaged. For proteomics, pregel solution is first buffered with 10×PBS, then precipitated with chloroform-methanol-water. Dried pregel is then processed with typsin/Lys-C. The protein precipitation is then ready for Liquid Chromatography-Mass Spectrometry/MS (LC-MS/MS) analysis via the Yale Keck Foundation Resources Lab (adapted from Jeong, Sang Young, et al. “Thrombospondin-2 secreted by human umbilical cord blood-derived mesenchymal stem cells promotes chondrogenic differentiation.” Stem Cells 31.10 (2013): 2136-2148).

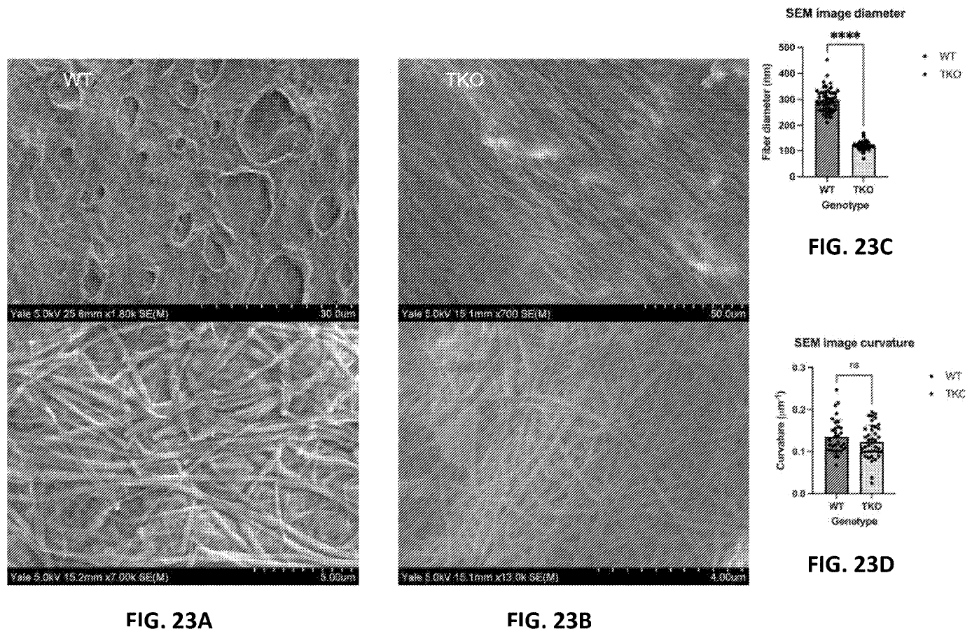

Preliminary SEM images of cartilage dECM hydrogels are shown in FIGS. 23A-23D. In cartilage hydrogels, the organization of ECM fibrils from collagen self-assembly is distinct from what is observed in other tissues such as skin. Namely, layered structures are observed with collagen nanofiber ‘outposts’ in SEM images. This layered structure, which is absent from skin and muscle hydrogels, might be assemblies of proteoglycans. TKO cartilage hydrogels show similar structures while having thinner collagen nanofibers than WT. This observation validates the hypothesized influence by the loss of TSP on cartilage tissue ECM organization. Quantification on fibers shows a larger diameter and comparable curvature in WT cartilage dECM hydrogels.

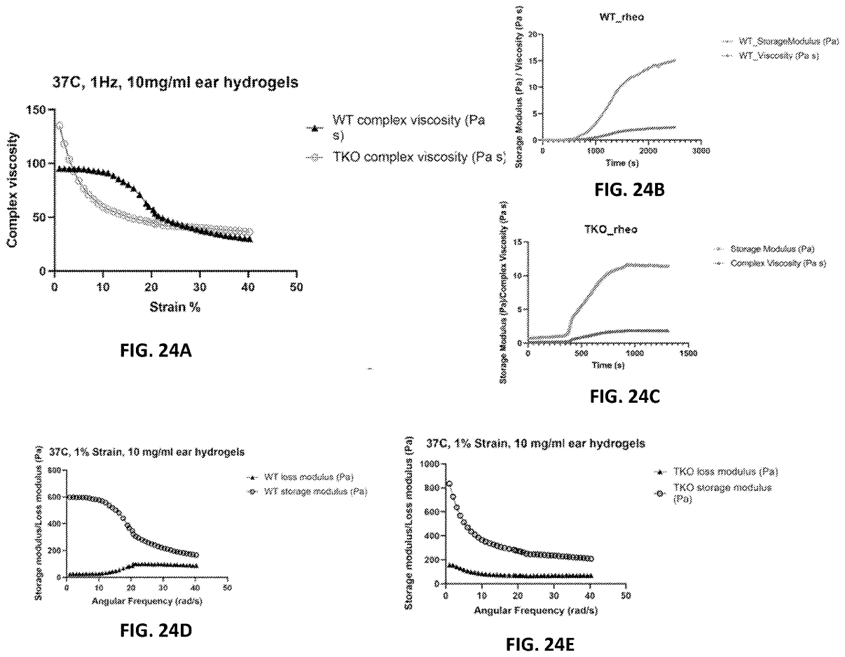

Preliminary rheometry data on pig ear hydrogels are shown in FIGS. 24A-24E. Frequency sweep shows different changes in storage modulus behavior between genotypes. There is a limited range of frequency in which WT storage modulus is larger than that of TKO, emphasizing proper control of experimental condition. Temperature sweep on both genotype pregels shows an increase in storage modulus and complex viscosity with increasing temperature. The maximum storage modulus of WT is larger than TKO, which is consistent with results from skin hydrogels. This validates the hypothesis that TKO dECM hydrogel is more fragile than WT.

Evaluation of Cartilage dECM-Derived Hydrogel Interaction with Cells

Testing and cross-comparison of a cell-free dECM scaffold, are best compensated by testing the interaction between the dECM network and cells in vitro. Cell lines and primary cells we reutilized to study cell-on-pregel, cell-on-hydrogel, and cell-in-hydrogel interactions. Basic cell morphology, viability, proliferation and transmigration were tested. Chondrogenic marker expression of cells interacting with cartilage dECM was tested by quantification of relevant gene expression. Additionally, a cell-free scaffold can be modified into a delivery system by loading hydrogel with external agents, including nanoparticles/microspheres capable of carrying growth factors.

Osteochondral cells have different interactions and behavior in WT and TKO hydrogels, with the latter improving cell spreading, migration and differentiation. It is anticipated that inclusion of external components, such as nano/microparticles, will not alter the favorable properties of hydrogels. The ‘enhanced’ hydrogel system could promote a more favorable environment for cartilage regeneration. This part of the project strives for a comprehensive understanding of TKO's influence on hydrogel-cell interaction, providing foundation and rationale for in vivo experiments. It also probes enhancing hydrogel by agent modification.

### Identification of Cell-Hydrogel Interaction and Chondrogenic Potential of WT and TKO Hydrogels.

dECM retains essential features of a native environment that favors cell proliferation. Hydrogels derived from decellularized animal tissues provide a permissive environment for cells to adhere, stretch and grow. dECM can modulate cell behavior by regulating molecular pathways with mechanotransduction and small molecule signaling factors. Based on available information on TSP2's role in bone and cartilage development, cells interacting with hydrogels have different morphology and gene expression from cells on an uncoated tissue culture well bottom. Namely, TKO hydrogel enhances chondrogenesis and bmMSC differentiation.

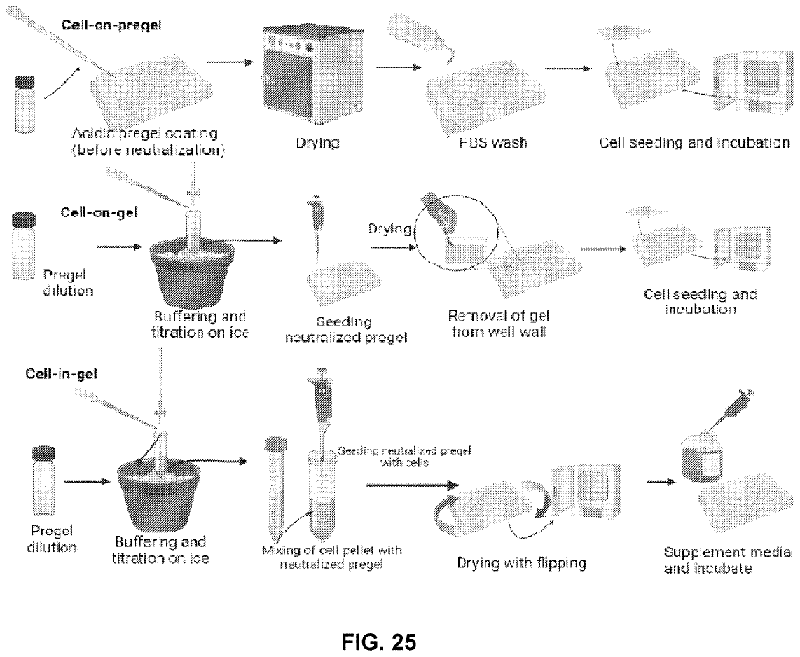

There are three in vitro cell-gel interaction systems of interest in this sub-aim. Each is set up with different protocols but evaluated with comparable methodologies. Rat tail collagen I is used in every system together with fabricated hydrogels as a separate positive control and undergoes the same dilution, neutralization and gelation protocol. Cells seeded on an uncoated tissue culture well bottom serve as a negative control. Cell lines of interest for this sub-aim are MC3T3-WT and ATDC5. Experiments would move to primary human chondrocytes and bmMSCs once results are obtained from cell lines. The three in vitro systems and their fabrication are explained as follows, with a simplified diagram of the workflow shown in FIG. 25.
(1) Cell-on-pregel: This was an experimental set up to observe cell behavior on monomeric collagen surface. Acidic pregel from both genotypes was diluted to 50-100 μg/ml in protein concentration. Well plates were coated with diluted pregel and then completely dried in a non-humidified incubator at 37° C. Remaining acid was washed away with PBS. Monomeric collagen was retained on the plate and ready for cell culture.
(2) Cell-on-gel: This was an experimental set up to observe cell behavior on fibrillar collagen surface. Pregel was diluted to 1 mg/ml in protein concentration. Buffering and titration were performed on ice for both genotypes and collagen I. Neutralized pregel is immediately pipetted into well plates to fully cover plate bottom and gelated in a non-humidified incubator at 37° C. for 30 minutes. Gels are gently removed from plate bottom with scalpel to prevent contraction-induced growth inhibition of cells. The gel surface is thereby ready for cell culture.
(3) Cell-in-gel: This is an experimental set up to observe cell behavior in 3D. Diluted pregel is buffered and titrated on ice. Pregel was immediately mixed with concentrated cell-media suspension with a concentration of 200K cell/ml pregel. Cell-pregel mixture was pipetted to well plates and incubated in a humidified incubator for 30 minutes, with manual flipping every 2-3 minutes to prevent cell sedimentation to plate bottom. Post gelation, gels were gently removed from plate bottom. Appropriate volume of media was then added to culture cells.

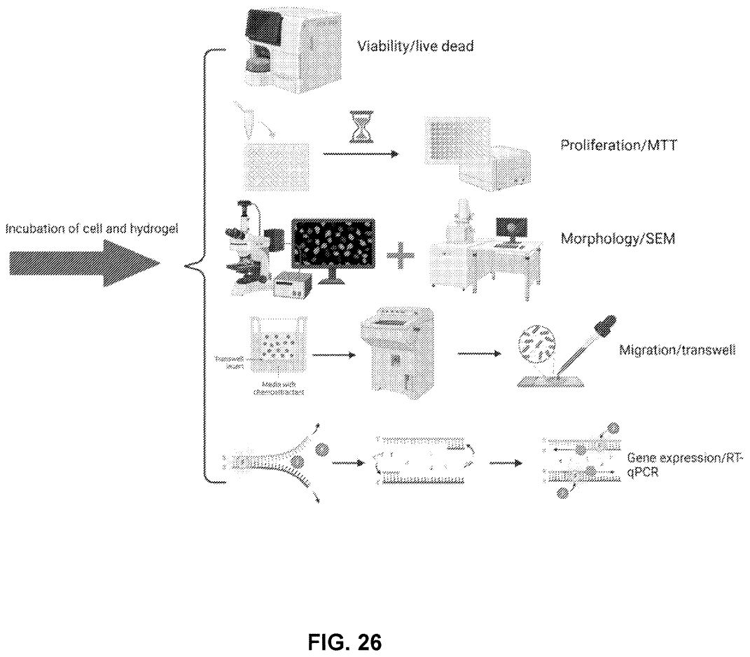

For all three setups, four conditions are devised to evaluate cell behavior and cell interaction with hydrogels, as illustrated in FIG. 26. Cell viability is evaluated by live/dead staining with nucBlue and nucGreen up until 72 hours post seeding. Cell proliferation is measured in cell-on-pregel and cell-on-gel groups with MTT assay up until 72 hours post seeding. Cell morphology is probed with DAPI and rhodamine staining at multiple timepoints up until 7 days. Cell morphology is confirmed by prepping individual wells for SEM imaging. Cell migration into gel is measured with transwell assay: hydrogel is fabricated inside transwell inserts. Cells are seeded as a monolayer on top of the hydrogel. Serum-free culture media is pipetted into the inserts while FBS-supplemented media is pipetted into bottom wells. Scaffolds are harvested at 12, 24 and 48 hours, fixed, cryosectioned and stained with immunocytochemistry (ICC) to visualize cell-hydrogel interaction. Finally, gene expression changes when cultured with hydrogel are probed. Cells are harvested at 72 hours, 7 days and 10 days post seeding, and undergo standard RNA extraction protocol. Reverse transcription qPCR is then performed to identify expression of chondrogenesis (e.g. Col II, Sox9 and aggrecan) and osteogenesis markers (e.g. Runx2, Opn and Ocn).

In all experimental groups with pregel or hydrogel interacting with cells, cell viability or proliferation are not be significantly affected compared to seeding on uncoated wells. Morphologically, cells interacting with the hydrogel are shown to have better spreading. Hydrogel derived from TKO cartilage dECM is expected to have better cell migration into the gel due to the loosely organized ECM. In addition, cells interacting with cartilage dECM hydrogel are expected to show increased chondrogenic marker expression as measured by RT-qPCR.

Investigation of Hydrogel Property Changes with Loading of External Agents

Hydrogels can be applied on their own; they can also be developed into a delivery system to enhance their therapeutic potential. While incorporating external agents such as cells and small molecules, it is expected that desirable properties of decellularized ECM are preserved to maintain favorable cell-ECM interactions. To setup for the basis of in vivo experiments, it is essential to know if a growth factor delivery system could further improve the performance of the hydrogel in accelerating cartilage regeneration.

In this sub-aim, synthesized PLGA particles are incorporated into WT and TKO hydrogels. Empty, dye-loaded and growth factor loaded particles are synthesized with a double-emulsion method adapted from Bahal, Raman, et al. “In vivo correction of anaemia in β-thalassemic mice by γPNA-mediated gene editing with nanoparticle delivery.” Nature communications 7.1 (2016): 13304. Chloroform is used as the organic solvent and 4% (concentrated)/0.3% (diluted) PVA in dH2O are used as the aqueous solvents. Firstly, 50:50 or 75:25 PLGA is solubilized in the organic phase. Growth factors (e.g. BMPs, TGFβ) are then added into the organic phase, while DiD dye is loaded into a group of PLGA particles for confirmation of successful loading. The mixture is then added into a more concentrated aqueous phase. After brief sonication, the WI/O (water 1/organic) mixture is poured into a second, more diluted organic phase and stirred at room temperature for 3 hours. The resulting solution is centrifuged at 4 degrees at 3000 RPM for 3×15 mins, washing with dH2O each time. The particle pellet is then lyophilized.

After synthesis, particles are lyophilized and weighed for yield calculation. Particles are characterized by direct light scattering (DLS) and nanoparticle tracking analysis (NTA) for their size distribution, and SEM for their shape homogeneity. Encapsulation of DiD is confirmed by fluorescence microscopy. Release profile of growth factors from particles is measured by dialysis followed with enzyme-linked immunosorbent assay (ELISA). For hydrogel incorporation, particles are mixed with neutralized pregel on ice with a variety of concentrations (tentatively between 1×10⁵ and 1×10⁶ particles/ml pregel), and then gelated. Hydrogel property changes are quantified by approaches such as measurements of gelation time, rheometry. SEM, and in vitro toxicity to cells.

In Vivo Assessment of Cartilage Regeneration with dECM Hydrogels

With a deeper understanding of its properties and a confirmation of its biocompatibility and capability of acting as a delivery system, an in vivo study is proposed in this aim with a murine model to determine the translational potential of the hydrogel.

WT and TKO cartilage dECM are expected to show different biophysical properties and interactions with cells, therefore it is reasonable to hypothesize that WT and TKO mice have different responses to artificially induced cartilage defects, with TKO mice having a more rapid healing but slower cartilage regeneration. Moreover, within WT mice, WT and TKO cartilage dECM hydrogels applied to a defect model are hypothesized to have regenerative property, with TKO behaving better than a gold standard hyaluronic acid injection.

Identification of Differences with Cartilage Defect Regeneration in WT and TKO Animals

Full thickness cartilage defect is a wound model involving tissue inflammation with very limited regeneration. The defect results in significant reduction in local chondrocyte and collagen II, as well as compromised biomechanics, which resemble pathological features in human OA. Healing in WT animals is dominated by spontaneous migration of bmMSC from subchondral marrow to the defect site, which is considerably slow. If left untreated, the cartilage degeneration will lead to loss-of-function of the entire joint.

TKO animals are known to have a favorable wound healing profile in skin wound models because of their more permissive ECM and increased angiogenesis. The TKO genotype is also associated with less chronic inflammation and fibrosis post injury. However, TKO animals are more effective in forming bones and less effective in forming cartilage as suggested in literature. Whether cartilage damage will result in a more rapid, bony replacement in TKO animals is unclear. Therefore, healing from a full thickness cartilage defect in WT and TKO animals is worth investigation. This sub-aim performs a set of proof-of-concept experiments that explores the role of TSP2 in chondrogenesis.

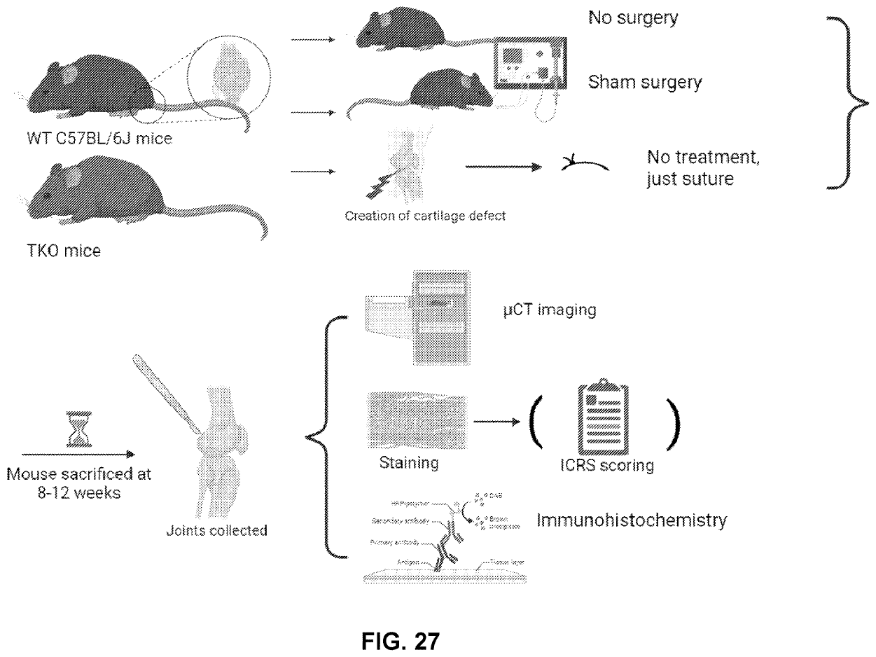

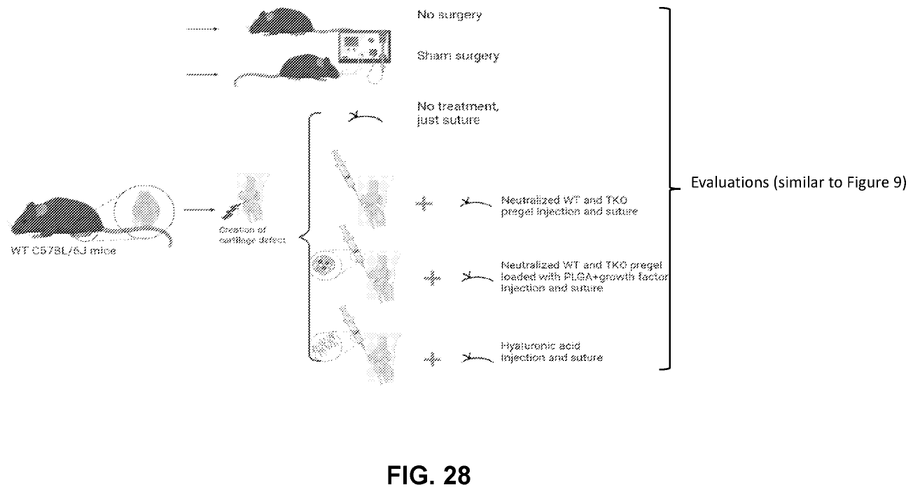

N=4 young mice (approx. 3 months in age) are chosen from each genotype (WT C57BL/6J and TKO) due to the inability of older mice to heal articular cartilage damage. Surgery is carried out according to a previously reported protocol (Matsuoka, Masatake, et al. “An articular cartilage repair model in common C57BI/6 mice.” Tissue Engineering Part C: Methods 21.8 (2015): 767-772) and is illustrated in FIG. 27. Six groups are intended: no surgery, sham control and surgery with no treatment, for both WT and TKO. Animals are sacrificed from 6 to 12 weeks post surgery. Knee joints are imaged with μCT to visualize defect sites. In addition, the femur head is collected, paraffin embedded, and sectioned in axial direction for staining with H&E, Alcian Blue (for proteoglycans) and Safranin O (for GAGs), as well as immunohistochemistry (IHC) against type I/II collagens (for cartilage-specific proteins) and Mac3 (for inflammatory macrophages). In addition, deidentified tissue sections collected at 8 weeks are scored according to International Cartilage Regeneration and joint preservation Society (ICRS) standard.

Evaluation of Regenerative Potential of WT and TKO Cartilage dECM Hydrogels in WT Animals

Application of biodegradable dECM hydrogel has demonstrated accelerated wound healing and decreased inflammation in a variety of murine models. Specifically, dECM hydrogel from TKO animals shows favorable behavior compared to WT. Compared to other tissues, the avascular and hypocellular nature of cartilage slows down regeneration when injury is introduced. This might also attenuate the potential pro-angiogenic effect of TKO hydrogels. In the interest of evaluating wound healing effect of a novel hydrogel system, it is necessary to introduce hydrogel to WT animals in a cartilage defect model.

Experimental setup is illustrated in FIG. 28. WT mice are separated into 6 groups, with N=4 for each group: Negative control (no treatment), gold standard (HA injection), WT and TKO neutralized pregel, and WT and TKO neutralized pregel loaded with PLGA particles and growth factors. Full thickness cartilage defects are created with methods described. Simultaneously during surgery, 10 mg dECM/ml cartilage dECM pregels from WT or TKO pigs are neutralized on ice. A syringe with 23G needle tip is then used to inject pregel from WT or TKO to completely fill the defect area. Hydrogel is allowed to gelate in situ. HA injections are performed in consistency with clinical practices-medical grade sterile 10 mg/ml HA solution is applied to the defect area. Post surgery, animals are treated with antibiotics and analgesics, and monitored for dehydration. Tissue samples are collected and evaluated as described.

Completion of the project will push the frontier of dECM hydrogel systems as the workflow of deriving cartilage hydrogels could be extrapolated to a variety of cartilage tissues. In addition, the effects of TSP2 in cartilage ECM organization and function, which have remained elusive in existing literature, will be elucidated. It will further our understanding of cells interacting within the cartilage niche by investigating the influence of dECM hydrogel on chondrocyte and osteoblast behavior. It will explore possibilities of ex vivo bone formation by growing bone cells in a scaffold. It will also investigate feasibility of improving naturally-derived hydrogel performance by loading with growth-factor encapsulated PLGA particles. The project introduces translational potential to the project and, if successful, will suggest a preliminary regime for osteoarthritis regenerative medicine using in situ hydrogel injection.

### Enumerated Embodiments

The following enumerated embodiments are provided, the numbering of which is not to be construed as designating levels of importance.
Embodiment 1 provides a method for promoting tissue regeneration in a subject in need thereof, the method comprising administering to the subject a decellularized extracellular matrix (ECM) of a tissue, wherein the tissue lacks functional thrombospondin-2 (TSP2).
Embodiment 2 provides the method of Embodiment 1, wherein the tissue is a musculoskeletal tissue.
Embodiment 3 provides the method of Embodiment 1, wherein the ECM is formulated as a hydrogel.
Embodiment 4 provides the method of any one of Embodiments 1-3, wherein the tissue is a muscle, a cartilage, a connective tissue, a tendon, a ligament, or a bone.
Embodiment 5 provides the method of any one of the previous Embodiments, wherein the administering is performed at one or more treatment site(s) of the subject.
Embodiment 6 provides the method of any one of the previous Embodiments, wherein the administering is performed by at least one method selected from the group consisting of subcutaneous, intramuscular, intraosseous, and topical administration.
Embodiment 7 provides the method of any one of the previous Embodiments, wherein cells of the tissue lacking functional TSP2 comprise: (a) a TSP2-null knockout allele; and/or (b) suppressed TSP2 gene expression.
Embodiment 8 provides the method of Embodiment 7, wherein the TSP2 null knockout allele or suppressed TSP2 gene expression is obtained via a genetic engineering technique comprising a nuclease selected from the group consisting of a clustered regularly interspaced short palindromic repeats (CRISPR) associated nuclease, a transcription activator-like effector nuclease (TALEN), and a zinc-finger nuclease.
Embodiment 9 provides the method of Embodiment 7, wherein cells of the tissue lacking functional TSP2 comprise an inhibitory RNA molecule which suppresses TSP2 gene expression.
Embodiment 10 provides the method of Embodiment 9, wherein the inhibitory RNA molecule is selected from the group consisting of: an RNA interference (RNAI) RNA, a short bairpin RNA (shRNA), a small interfering RNA (siRNA), a trans-acting siRNA (tasiRNA), a micro RNA (miRNA), an antisense RNA (asRNA), a long noncoding RNA (lncRNA), a CRISPR RNA (crRNA), a trans-activating crRNA (tracrRNA), a guide RNA (gRNA), a single guide RNA (sgRNA), a double-stranded RNA (dsRNA), a ribozyme, and any combination thereof.
Embodiment 11 provides the method of any one of the previous Embodiments, wherein the method enhances at least one biological response at the treatment site, as compared to a site administered a decellularized ECM originating from a tissue comprising functional TSP2, or compared to an untreated site.
Embodiment 12 provides the method of Embodiment 11, wherein the biological response at the treatment site is selected from the group consisting of cellular migration towards the treatment site, cellular invasion of the treatment site, vascular growth and maturation, innervation, angiogenesis, and wound repair.
Embodiment 13 provides the method of any one of the previous Embodiments, wherein the tissue lacking functional TSP2 is muscle and wherein the subject suffers from at least one condition selected from the group consisting of a muscle injury, a myopathy, a genetic myopathy, an inflammatory myopathy, an endocrine myopathy, a neuromuscular disorder, amyotrophic lateral sclerosis, Charcot-Marie-Tooth disease, multiple sclerosis, a muscular dystrophy, Duchenne muscular dystrophy, Becker muscular dystrophy, Limb Girdle muscular dystrophy, spinal muscular atrophy, Guillain-Barre Syndrome, Chronic Inflammatory Demyelinating Polyneuropathy, Multifocal Motor Neuropathy, Myasthenia Gravis, Pompe's Disease, Drop Head Syndrome (floppy head syndrome), multisystemic smooth muscle dysfunction syndrome, peripheral vascular disease, congenital heart disease, a cardiomyopathy, coronary artery disease, heart attack, heart valve disease, hypertension, hernia, type 1 diabetes, type 2 diabetes, alcohol use, and tobacco use.
Embodiment 14 provides the method of any one of the previous Embodiments, wherein the tissue lacking functional TSP2 is bone and wherein the subject suffers from at least one condition selected from the group consisting of a bone injury, a bone disorder, osteoporosis, osteopetrosis, osteonecrosis, osteogenesis imperfecta, osteoarthritis, rheumatoid arthritis, type 1 diabetes, type 2 diabetes, lupus, celiac disease, hyperthyroidism, infection of bone or joint, Paget's Disease of Bone, fibrous dysplasia, tobacco use, and weight loss surgery.
Embodiment 15 provides the method of any one of the previous Embodiments, wherein the tissue lacking functional TSP2 is cartilage and wherein the subject suffers from at least one condition selected from the group consisting of osteoarthritis, rheumatoid arthritis, avascular necrosis, costochondritis, a fracture, lupus, Maffucci syndrome, osteoporosis, achondroplasia, conclusion, and herniation.
Embodiment 16 provides the method of any one of the previous Embodiments, wherein the subject is a mammal.
Embodiment 17 provides the method of any one of the previous Embodiments, wherein the tissue originates from a mammal selected from the group consisting of a mouse, a pig, a non-human primate, and a human.
Embodiment 18 provides the method of any one of the previous Embodiments, wherein the subject is a human.
Embodiment 19 provides the method of any one of the previous Embodiments, wherein the tissue lacking functional TSP2 is autologous, allogeneic, or xenogeneic relative to the subject.
Embodiment 20 provides the method of any one of the previous Embodiments, wherein the tissue lacking functional TSP2 is not skin.
Embodiment 21 provides the method of any one of the previous Embodiments, wherein the ECM is formulated with at least one therapeutic agent.
Embodiment 22 provides the method of Embodiment 21, wherein the at least one therapeutic agent is selected from the group consisting of an immunosuppressive agent, an anti-inflammatory agent, an antimetabolite, an antibiotic, an antibody, a growth factor, a cytokine, a gene therapy, an immunomodulator, and any combination thereof.
Embodiment 23 provides a decellularized extracellular matrix (ECM) of a tissue, wherein the tissue lacks functional thrombospondin-2 (TSP2).
Embodiment 24 provides the decellularized ECM of Embodiment 23, wherein the tissue is a musculoskeletal tissue.
Embodiment 25 provides the decellularized ECM of Embodiment 23 or 24, wherein the ECM is formulated as a hydrogel.
Embodiment 26 provides the decellularized ECM of any one of Embodiments 23-25, wherein the tissue is muscle, cartilage, a connective tissue, a tendon, a ligament, or a bone.
Embodiment 27 provides the decellularized ECM of any one of Embodiments 23-26, wherein cells of the tissue lacking functional TSP2 comprise: (a) a TSP2-null knockout allele; and/or (b) suppressed TSP2 gene expression.
Embodiment 28 provides the decellularized ECM of Embodiment 27, wherein the TSP2 null knockout allele or suppressed TSP2 gene expression is obtained via a genetic engineering technique comprising a nuclease selected from the group consisting of a clustered regularly interspaced short palindromic repeats (CRISPR) associated nuclease, a transcription activator-like effector nuclease (TALEN), and a zinc-finger nuclease.
Embodiment 29 provides the decellularized ECM of Embodiment 27, wherein cells of the tissue lacking functional TSP2 comprise an inhibitory RNA molecule which suppresses TSP2 gene expression.
Embodiment 30 provides the decellularized ECM of Embodiment 29, wherein the inhibitory RNA molecule is selected from the group consisting of: an RNA interference (RNAi) RNA, a short hairpin RNA (shRNA), a small interfering RNA (siRNA), a trans-acting siRNA (tasiRNA), a micro RNA (miRNA), an antisense RNA (asRNA), a long noncoding RNA (lncRNA), a CRISPR RNA (crRNA), a trans-activating crRNA (tracrRNA), a guide RNA (gRNA), a single guide RNA (sgRNA), a double-stranded RNA (dsRNA), a ribozyme, and any combination thereof.
Embodiment 31 provides the decellularized ECM of any one of the previous Embodiments, wherein the tissue lacking functional TSP2 originates from a mammal selected from the group consisting of a mouse, a pig, a non-human primate, and a human.
Embodiment 32 provides the decellularized ECM of any one of the previous Embodiments, wherein the tissue lacking functional TSP2 is not skin.
Embodiment 33 provides the decellularized ECM of any one of the previous Embodiments, wherein the ECM is formulated with at least one therapeutic agent.
Embodiment 34 provides the decellularized ECM of Embodiment 33, wherein the at least one therapeutic agent is selected from the group consisting of an immunosuppressive agent, an anti-inflammatory agent, an antimetabolite, an antibiotic, an antibody, a growth factor, a cytokine, a gene therapy, an immunomodulator, and any combination thereof.
Embodiment 35 provides a composition comprising the decellularized ECM of any one of claims and a pharmaceutically acceptable carrier.
Embodiment 36 provides a method for preparing a decellularized extracellular matrix (ECM), the method comprising: (1) obtaining or having obtained a tissue lacking functional thrombospondin-2 (TSP2) from a mammal; and (ii) decellularizing the tissue to generate the decellularized ECM.
Embodiment 37 provides the method of Embodiment 36, wherein the tissue is a musculoskeletal tissue.
Embodiment 38 provides the method of Embodiment 36, wherein the tissue is a muscle, a cartilage, a connective tissue, a tendon, a ligament, or a bone.
Embodiment 39 provides the method of any one of Embodiments 36-38, wherein the tissue lacking functional TSP2 is muscle and wherein decellularizing the tissue comprises treating the muscle at room temperature sequentially with (i) an aqueous Trypsin-EDTA solution for about 5-7 hours; (ii) an aqueous H₂O₂ solution for about 15 minutes: (iii) an aqueous Triton X-100/EDTA/Tris solution for about 5-7 hours; (iv) a fresh aqueous Triton X-100/EDTA/Tris solution for about 5-15 hours; and (v) an aqueous Triton X-100/sodium deoxycholate (SDC) solution for about 5-7 hours.
Embodiment 40 provides the method of any one of Embodiments 36-39, wherein the tissue is bone, wherein the method further comprises demineralizing the bone to generate demineralized bone matrix (DBM), and wherein decellularizing the tissue comprises decellularizing the DBM.
Embodiment 41 provides the method of Embodiment 40, wherein demineralizing the bone comprises incubating the fragmented bone with agitation in an aqueous acid solution at room temperature for about 20-28 hours, optionally wherein the aqueous acid solution comprises approximately 0.5 N HCl.
Embodiment 42 provides the method of Embodiment 40 or Embodiment 41, wherein decellularizing the DBM comprises incubating the DBM with agitation with an aqueous trypsin-EDTA solution at approximately 37° C. for about 20-28 hours.
Embodiment 43 provides the method of any one of Embodiments 36-42, further comprising fragmenting the bone.
Embodiment 44 provides the method of any one of Embodiments 36-43, wherein the tissue is cartilage, wherein the method further comprises prior to decellularization: i) removing skin and fat remnants, ii) sterilizing with ethanol, and iii) oxidizing the sample with about 3% hydrogen peroxide.
Embodiment 45 provides the method of any one of Embodiments 36-44, wherein the decellularizing comprises about 1% Triton solution/EDTA/Tris for about 36 hours.
Embodiment 46 provides the method of any one of Embodiments 36-45, further comprising washing the sample with ethanol/peracetic acid/ddH2O.
Embodiment 47 provides the method of any one of Embodiments 36-46, further comprising incubating the sample in fetal bovine serum (FBS) free culture media with about 1% antibiotics overnight and then lyophilized.
Embodiment 48 provides the method of any one of Embodiments 36-47, further comprising formulating the decellularized ECM as a hydrogel.
Embodiment 49 provides the method of any one of Embodiments 36-48, wherein cells of the tissue lacking functional TSP2 comprise: (a) a TSP2-null knockout allele; and/or (b) suppressed TSP2 gene expression.
Embodiment 50 provides the method of Embodiment 49, wherein the TSP2 null knockout allele or suppressed TSP2 gene expression is obtained via a genetic engineering technique comprising a nuclease selected from the group consisting of a clustered regularly interspaced short palindromic repeats (CRISPR) associated nuclease, a transcription activator-like effector nuclease (TALEN), and a zinc-finger nuclease.
Embodiment 51 provides the method of Embodiment 50, wherein cells of the tissue lacking functional TSP2 comprise an inhibitory RNA molecule which suppresses TSP2 gene expression.
Embodiment 52 provides the method of Embodiment 51, wherein the inhibitory RNA molecule is selected from the group consisting of: an RNA interference (RNAI) RNA, a short hairpin RNA (shRNA), a small interfering RNA (siRNA), a trans-acting siRNA (tasiRNA), a micro RNA (miRNA), an antisense RNA (asRNA), a long noncoding RNA (lncRNA), a CRISPR RNA (crRNA), a trans-activating crRNA (tracrRNA), a guide RNA (gRNA), a single guide RNA (sgRNA), a double-stranded RNA (dsRNA), a ribozyme, and any combination thereof.
Embodiment 53 provides the method of any one of Embodiments 36-52, wherein the mammal is selected from the group consisting of a mouse, a pig, and a human.
Embodiment 54 provides the method of any one of Embodiments 36-53, wherein the method further comprises formulating the decellularize ECM with at least one therapeutic agent.
Embodiment 55 provides the method of Embodiment 54, wherein the at least one therapeutic agent is selected from the group consisting of an immunosuppressive agent, an anti-inflammatory agent, an antimetabolite, an antibiotic, an antibody, a growth factor, a cytokine, a gene therapy, an immunomodulator, and any combination thereof.

### Other Embodiments

The disclosures of each and every patent, patent application, and publication cited herein are hereby incorporated herein by reference in their entirety.

While the present invention has been disclosed with reference to specific embodiments, it is apparent that other embodiments and variations of the present invention may be devised by others skilled in the art without departing from the true spirit and scope of the invention. The appended claims are intended to be construed to include all such embodiments and equivalent variations.

1. A method for promoting tissue regeneration in a subject in need thereof, the method comprising administering to the subject a decellularized extracellular matrix (ECM) of a tissue, wherein the tissue lacks functional thrombospondin-2 (TSP2).

2. (canceled)

3. The method of claim 1, wherein the ECM is formulated as a hydrogel.

4. The method of claim 1, wherein the tissue is a muscle, a cartilage, a connective tissue, a tendon, a ligament, or a bone.

5. The method of claim 1, wherein the administering is performed at one or more treatment site(s) of the subject.

6. (canceled)

7. The method of claim 1, wherein cells of the tissue lacking functional TSP2 comprise:
(a) a TSP2-null knockout allele; and/or
(b) suppressed TSP2 gene expression.

8-10. (canceled)

11. The method of claim 1, wherein the method enhances at least one biological response at the treatment site, as compared to a site administered a decellularized ECM originating from a tissue comprising functional TSP2, or compared to an untreated site.

12-15. (canceled)

16. The method of claim 1, wherein the subject is a mammal.

17-18. (canceled)

19. The method of claim 1, wherein the tissue lacking functional TSP2 is autologous, allogeneic, or xenogeneic relative to the subject.

20-22. (canceled)

23. A decellularized extracellular matrix (ECM) of a tissue, wherein the tissue lacks functional thrombospondin-2 (TSP2).

24. (canceled)

25. The decellularized ECM of claim 23, wherein the ECM is formulated as a hydrogel.

26. The decellularized ECM of claim 23, wherein the tissue is muscle, cartilage, a connective tissue, a tendon, a ligament, or a bone.

27. The decellularized ECM of claim 23, wherein cells of the tissue lacking functional TSP2 comprise:
(a) a TSP2-null knockout allele; and/or
(b) suppressed TSP2 gene expression.

28-35. (canceled)

36. A method for preparing a decellularized extracellular matrix (ECM), the method comprising:
(i) obtaining or having obtained a tissue lacking functional thrombospondin-2 (TSP2) from a mammal; and
(ii) decellularizing the tissue to generate the decellularized ECM.

37. (canceled)

38. The method of claim 36, wherein the tissue is a muscle, a cartilage, a connective tissue, a tendon, a ligament, or a bone.

39-42. (canceled)

43. The method of claim 36, further comprising fragmenting the bone.

44-45. (canceled)

46. The method of claim 36, further comprising washing the sample with ethanol/peracetic acid/ddH2O.

47. The method of claim 36, further comprising incubating the sample in fetal bovine serum (FBS) free culture media with about 1% antibiotics overnight and then lyophilized.

48. The method of claim 36, further comprising formulating the decellularized ECM as a hydrogel.

49. The method of claim 36, wherein cells of the tissue lacking functional TSP2 comprise:
(a) a TSP2-null knockout allele; and/or
(b) suppressed TSP2 gene expression.

50-52. (canceled)

53. The method of claim 36, wherein the mammal is selected from the group consisting of a mouse, a pig, and a human.

54-55. (canceled)


SEQUENCE LISTING (30 sequences)

SEQ ID NO: 1 - DNA, 22 bp, synthetic construct
  source 1..22: mol_type = other DNA
tcaatgggactgcatatctgcc

SEQ ID NO: 2 - DNA, 22 bp, synthetic construct
  source 1..22: mol_type = other DNA
gccaaaatactaccagctcact

SEQ ID NO: 3 - DNA, 22 bp, synthetic construct
  source 1..22: mol_type = other DNA
caaccaacaagtgatattctcc

SEQ ID NO: 4 - DNA, 21 bp, synthetic construct
  source 1..21: mol_type = other DNA
gatccacactctccagctgca

SEQ ID NO: 5 - DNA, 25 bp, synthetic construct
  source 1..25: mol_type = other DNA
catcttctcaaaattcgagtgacaa

SEQ ID NO: 6 - DNA, 21 bp, synthetic construct
  source 1..21: mol_type = other DNA
tgggagtagacaaggtacaac

SEQ ID NO: 7 - DNA, 22 bp, synthetic construct
  source 1..22: mol_type = other DNA
tgacgtcactggagttgtacgg

SEQ ID NO: 8 - DNA, 21 bp, synthetic construct
  source 1..21: mol_type = other DNA
ggttcatgtcatggatggtgc

SEQ ID NO: 9 - DNA, 21 bp, synthetic construct
  source 1..21: mol_type = other DNA
gctcttactgactggcatgag

SEQ ID NO: 10 - DNA, 20 bp, synthetic construct
  source 1..20: mol_type = other DNA
cgcagctctaggagcatgtg

SEQ ID NO: 11 - DNA, 22 bp, synthetic construct
  source 1..22: mol_type = other DNA
ctccaagccaaagtccttagag

SEQ ID NO: 12 - DNA, 22 bp, synthetic construct
  source 1..22: mol_type = other DNA
aggagctgtcattagggacatc

SEQ ID NO: 13 - DNA, 20 bp, synthetic construct
  source 1..20: mol_type = other DNA
ttcaccaccatggagaaggc

SEQ ID NO: 14 - DNA, 20 bp, synthetic construct
  source 1..20: mol_type = other DNA
ggcatggactgtggtcatga

SEQ ID NO: 15 - DNA, 20 bp, synthetic construct
  source 1..20: mol_type = other DNA
cggaatgcctctgctgttat

SEQ ID NO: 16 - DNA, 22 bp, synthetic construct
  source 1..22: mol_type = other DNA
tgtgaagacggttatggtcaag

SEQ ID NO: 17 - DNA, 23 bp, synthetic construct
  source 1..23: mol_type = other DNA
tctgatgaactggtcactgattt

SEQ ID NO: 18 - DNA, 22 bp, synthetic construct
  source 1..22: mol_type = other DNA
ctcggccatcatatgtgtctac

SEQ ID NO: 19 - DNA, 18 bp, synthetic construct
  source 1..18: mol_type = other DNA
tgcagagtccagcaaagg

SEQ ID NO: 20 - DNA, 21 bp, synthetic construct
  source 1..21: mol_type = other DNA
cccagccattgatacaggtag

SEQ ID NO: 21 - DNA, 23 bp, synthetic construct
  source 1..23: mol_type = other DNA
ggtgtgaaccatgagaagtatga

SEQ ID NO: 22 - DNA, 22 bp, synthetic construct
  source 1..22: mol_type = other DNA
gagtccttccacgataccaaag

SEQ ID NO: 23 - DNA, 21 bp, synthetic construct
  source 1..21: mol_type = other DNA
gccacttaccacagagctatt

SEQ ID NO: 24 - DNA, 21 bp, synthetic construct
  source 1..21: mol_type = other DNA
gaggcgatcagagaacaaact

SEQ ID NO: 25 - DNA, 21 bp, synthetic construct
  source 1..21: mol_type = other DNA
acgacgatgatgacgatgatg

SEQ ID NO: 26 - DNA, 22 bp, synthetic construct
  source 1..22: mol_type = other DNA
gactcatccgaatggtgagatt

SEQ ID NO: 27 - DNA, 20 bp, synthetic construct
  source 1..20: mol_type = other DNA
gtgagcttaaccctgcttgt

SEQ ID NO: 28 - DNA, 22 bp, synthetic construct
  source 1..22: mol_type = other DNA
agtgataccgtagatgcgtttg

SEQ ID NO: 29 - DNA, 20 bp, synthetic construct
  source 1..20: mol_type = other DNA
ttcaccaccatggagaaggc

SEQ ID NO: 30 - DNA, 20 bp, synthetic construct
  source 1..20: mol_type = other DNA
ggcatggactgtggtcatga